(19) United States
(12) Patent Application Publication
(10) Pub. No.: US 2023/0104113 A1
(43) Pub. Date: Apr. 6, 2023

(54) DELIVERY OF COMPOSITIONS COMPRISING CIRCULAR POLYRIBONUCLEOTIDES

(71) Applicant: Flagship Pioneering Innovations VI, LLC, Cambridge, MA (US)

(72) Inventors: Avak KAHVEJIAN, Lexington, MA (US); Nicholas McCartney PLUGIS, Duxbury, MA (US); Alexandra Sophie DE BOER, Somerville, MA (US); Erica Gabrielle WEINSTEIN, Newton, MA (US); Catherine CIFUENTES-ROJAS, Brookline, MA (US)

(21) Appl. No.: 17/795,943

(22) PCT Filed: Jan. 29, 2021
(86) PCT No.: PCT/US2021/015737

§ 371 Date: Jul. 28, 2022

Related U.S. Application Data
(60) Provisional application No. 63/126,472, filed on Dec. 16, 2020.
(60) Provisional application No. 62/967,548, filed on Jan. 29, 2020.

(51) Int. Cl. A61K 31/713; A61K 48/00; C12N 15/115

(52) U.S. Cl. CPC A61K 31/713; A61K 48/0016; C12N 15/115; C12N 2310/16; C12N 2310/532; C12N 2320/32

(57) ABSTRACT

This invention relates generally to delivery of circular polyribonucleotides and compositions thereof.

### CROSS-REFERENCE TO RELATED APPLICATIONS

The present application claims priority to and benefit from U.S. Provisional Application Nos. 62/967,548, filed Jan. 29, 2020 and 63/126,472, filed Dec. 16, 2020, the entire contents of each of which are herein incorporated by reference.

### BACKGROUND

Certain circular polyribonucleotides are ubiquitously present in human tissues and cells, including tissues and cells of healthy individuals.

### SUMMARY

This disclosure relates generally to systems and methods for delivery of compositions comprising circular polyribonucleotides, wherein the circular polyribonucleotide comprises a binding site. In some embodiments, the system is a parentaeral nucleic acid delivery system. In some embodiments, the parenteral nucleic acid delivery system comprises a parenterally acceptable diluent and is free of any carrier. In some embodiments, the parenteral nucleic acid delivery system comprises a carrier. In some embodiments, the method comprises delivering by parenteral administration a composition comprising the circular polyribonucleotide. Parenteral administration can be intravenous, intramuscular, ophthalmic or topical administration. In some embodiments, the composition comprises the circular polyribonucleotide and a parenterally acceptable diluent. In some embodiments, the composition comprises the circular polyribonucleotide, a parenterally acceptable diluent, and is free of any carrier. In some embodiments, the composition comprises the circular polyribonucleotide, a parenterally acceptable diluent, and a carrier. In some embodiments, the binding site comprises a sequence that binds a target. The circular polyribonucleotide can be translation incompetent.

In a first aspect, the invention features, a parenteral nucleic acid delivery system comprising a circular polyribonucleotide and a parenterally acceptable diluent, wherein the circular polyribonucleotide comprises a sequence that binds a target.

In some embodiments, the delivery system is free of any carrier. In some embodiments, the delivery system comprises a carrier. In some embodiments, the circular polyribonucleotide is in an amount effective to elicit a biological response in the subject. In some embodiments, the circular polyribonucleotide is in an amount effective to have a biological effect on a cell or tissue in the subject.

In some embodiments, the system is formulated for intravenous, intramuscular, ophthalmic or topical administration. In some embodiments, the system is formulated for in vitro or ex vivo delivery to a cell or tissue.

In some embodiments, the circular polyribonucleotide forms a complex with the target and the circular polyribonucleotide or the target is detectable at least 5 days after delivery.

In some embodiments, the target is selected from the group consisting of a nucleic acid molecule, a small molecule, a protein, a carbohydrate, and a lipid.

In some embodiments, the small molecule is an organic compound having a molecular weight of no more than 900 daltons and modulates a cellular process. In some embodiments, the small molecule is a drug. In some embodiments, the small molecule is a fluorophore. In some embodiments, the small molecule is a metabolite.

In some embodiments, the target is a gene regulation protein. In some embodiments, the gene regulation protein is a transcription factor. In some embodiments, the nucleic acid molecule is a DNA molecule or an RNA molecule. In some embodiments, the complex modulates gene expression. In some embodiments, the complex modulates directed transcription of a DNA molecule, epigenetic remodeling of a DNA molecule, or degradation of a DNA molecule. In some embodiments, the complex modulates degradation of the target, translocation of the target, or target signal transduction. In some embodiments, the gene expression is associated with pathogenesis of a disease or condition.

In some embodiments, the circular polyribonucleotide of the complex or the target of the complex is detectable at least 7, 8, 9, or 10 days after delivery. In some embodiments, the circular polyribonucleotide is present at least five days after delivery. In some embodiments, the circular polyribonucleotide is present at least 6, 7, 8, 9, or 10 days after delivery.

In some embodiments, the circular polyribonucleotide is an unmodified circular polyribonucleotide. In some embodiments, the circular polyribonucleotide has a quasi-double-stranded secondary structure. In some embodiments, the sequence is an aptamer sequence that has a secondary structure that binds the target. In some embodiments, the aptamer sequence further has a tertiary structure that binds the target.

In some embodiments, the cell is a eukaryotic cell. In some embodiments, the eukaryotic cell is an animal cell. In some embodiments, the eukaryotic cells is a pet cell. In some embodiments, the eukaryotic cell is a mammalian cell. In some embodiments, the eukaryotic cell is a human cell. In some embodiments, the eukaryotic cell is a livestock cell. In some embodiments, the circular polyribonucleotide lacks a poly-A sequence, lacks a replication element, lacks a free 3′ end, or lacks an RNA polymerase recognition motif, or any combination thereof. In some embodiments, the circular polyribonucleotide is a translation incompetent circular polyribonucleotide. In some embodiments, the circular polyribonucleotide further comprises an expression sequence. In some embodiments, the circular polyribonucleotide comprises a termination element or an IRES, or the combination thereof.

In a second aspect, the invention features the parenteral nucleic acid delivery system of any one of the preceding embodiments for use as a medicament or a pharmaceutical.

In a third aspect, the invention features the parenteral nucleic acid delivery system of any one of the preceding embodiments for use in a method of treatment of a human or animal body by therapy.

In a fourth aspect, the invention features the parenteral nucleic acid delivery system of any one of the preceding embodiments in the manufacture of a medicament or a pharmaceutical.

In a fifth aspect, the invention features the parenteral nucleic acid delivery system of any one of the preceding embodiments in the manufacture of a medicament or a pharmaceutical for treating a human or animal body by therapy.

### Definitions

The present invention will be described with respect to particular embodiments and with reference to certain figures but the invention is not limited thereto. Terms as set forth hereinafter are generally to be understood in their common sense unless indicated otherwise.

As used herein, the terms “circRNA” or “circular polyribonucleotide” or “circular RNA” are used interchangeably and mean a polyribonucleotide molecule that has a structure having no free ends (i.e., no free 3′ and/or 5′ ends), for example a polyribonucleotide molecule that forms a circular or endless structure through covalent or non-covalent bonds.

As used herein, the term “encryptogen” is a nucleic acid sequence of the circular polyribonucleotide that aids in reducing, evading, and/or avoiding detection by an immune cell and/or reduces induction of an immune response against the circular polyribonucleotide.

As used herein, the term “expression sequence” is a nucleic acid sequence that encodes a product, e.g., a peptide or polypeptide, or a regulatory nucleic acid. An exemplary expression sequence that codes for a peptide or polypeptide can comprise a plurality of nucleotide triads, each of which can code for an amino acid and is termed as a “codon”.

As used herein, the term “modified ribonucleotide” means any ribonucleotide analog or derivative that has one or more chemical modifications to the chemical composition of an unmodified natural ribonucleotide, such as a natural unmodified nucleotide adenosine (A), uridine (U), guanine (G), cytidine (C). In some embodiments, the chemical modifications of the modified ribonucleotide are modifications to any one or more functional groups of the ribonucleotide, such as, the sugar the nucleobase, or the internucleoside linkage (e.g. to a linking phosphate/to a phosphodiester linkage/to the phosphodiester backbone).

As used herein, the phrase “quasi-helical structure” is a higher order structure of the circular polyribonucleotide, wherein at least a portion of the circular polyribonucleotide folds into a helical structure.

As used herein, the phrase “quasi-double-stranded secondary structure” is a higher order structure of the circular polyribonucleotide, wherein at least a portion of the circular polyribonucleotide creates an internal double strand.

As used herein, the term “regulatory sequence” is a nucleic acid sequence that modifies an expression product.

As used herein, the term “repetitive nucleotide sequence” is a repetitive nucleic acid sequence within a stretch of DNA or throughout a genome. In some embodiments, the repetitive nucleotide sequence includes poly CA or poly TG (UG) sequences. In some embodiments, the repetitive nucleotide sequence includes repeated sequences in the Alu family of introns.

As used herein, the term “replication element” is a sequence and/or motifs useful for replication or that initiate transcription of the circular polyribonucleotide.

As used herein, the term “selective translation sequence” is a nucleic acid sequence that selectively initiates or activates translation of an expression sequence in the circular polyribonucleotide.

As used herein, the term “selective degradation sequence” means a nucleic acid sequence that initiates translation of an expression sequence in the circular polyribonucleotide.

As used herein, the term “stagger element” is a moiety, such as a nucleotide sequence, that induces ribosomal pausing during translation. In some embodiments, the stagger element is a non-conserved sequence of amino-acids with a strong alpha-helical propensity followed by the consensus sequence -D(V/I)ExNPG P, where x is any amino acid. In some embodiments, the stagger element may include a chemical moiety, such as glycerol, a non nucleic acid linking moiety, a chemical modification, a modified nucleic acid, or any combination thereof.

As used herein, the term “substantially resistant” is one that has at least 50%, 60%, 70%, 75%, 80%, 85%, 90%, 95%, 96%, 97%, 98% or 99% resistance as compared to a reference.

As used herein, the term “complex” means an association between at least two moieties (e.g., chemical or biochemical) that have an affinity for one another. For example, at least two moieties are a target (e.g., a protein) and a circular RNA molecule.

“Polypeptide” and “protein” are used interchangeably and means a polymer of two or more amino acids joined by a covalent bond (e.g., an amide bond). Polypeptides as described herein can include full length proteins (e.g., fully processed proteins) as well as shorter amino acid sequences (e.g., fragments of naturally-occurring proteins or synthetic polypeptide fragments). Polypeptides can include naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V) and non-naturally occurring amino acids (e.g., amino acids which is not one of the twenty amino acids commonly found in peptides synthesized in nature, including synthetic amino acids, amino acid analogs, and amino acid mimetics).

As used herein, the term “binding site” is a region of the circular polyribonucleotide that interacts with another entity, e.g., a chemical compound, a protein, a nucleic acid, etc. A binding site can comprise an aptamer sequence.

As used herein, the term “binding moiety” is a region of a target that can be bound by a binding site, for example, a region, domain, fragment, epitope, or portion of a nucleic acid (e.g., RNA, DNA, RNA-DNA hybrid), chemical compound, small molecule (e.g., drug), aptamer, polypeptide, protein, lipid, carbohydrate, antibody, virus, virus particle, membrane, multi-component complex, organelle, cell, other cellular moieties, any fragment thereof, and any combination thereof.

As used herein, the term “aptamer sequence” is a non-naturally occurring or synthetic oligonucleotide that specifically binds to a target molecule. Typically an aptamer is from 20 to 500 nucleotides. Typically an aptamer binds to its target through secondary structure rather than sequence homology.

As used herein, the term “small molecule” is an organic compound that has a molecular weight of no more than 900 daltons. A small molecule is capable of modulating a cellular process or is a fluorophore.

As used herein, the term “conjugation moiety” is a modified nucleotide comprising a functional group for use in a method of conjugation.

As used herein, the term “linear counterpart” is a polyribonucleotide molecule (and its fragments) having the same or similar nucleotide sequence (e.g., 100%, 95%, 90%, 85%, 80%, 75%, or any percentage therebetween of sequence similarity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence similarity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween of sequence similarity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further comprises a 5′ cap. In some embodiments, the linear counterpart further comprises a poly adenosine tail. In some embodiments, the linear counterpart further comprises a 3′ UTR. In some embodiments, the linear counterpart further comprises a 5′ UTR.

As used herein, the term “carrier” is a compound, composition, reagent, or molecule that facilitates the transport or delivery of a composition (e.g., a circular polyribonucleotide) into a cell by a covalent modification of the circular polyribonucleotide, via a partially or completely encapsulating agent, or a combination thereof. Non-limiting examples of carriers include carbohydrate carriers (e.g., an anhydride-modified phytoglycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

As used herein, the term “naked delivery” means a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that comprises a circular polyribonucleotide without covalent modification and is free from a carrier.

The term “diluent” means vehicle comprising an inactive solvent in which a composition described herein (e.g., a composition comprising a circular polyribonucleotide) may be diluted or dissolved. A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, or powdered sugar.

As used herein, the term “parenterally acceptable diluent” is a diluent used for parenteral administration of a composition (e.g., a composition comprising a circular polyribonucleotide).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1 illustrates an example circular polyribonucleotide molecular scaffold.

FIG. 2 illustrates an example trans-ribozyme circular polyribonucleotide.

FIG. 3 illustrates a schematic of protein expression by a circular polyribonucleotide.

FIG. 4 illustrates an example circular polyribonucleotide molecular scaffold for lipids, such as membranes.

FIG. 5A illustrates an example circular polyribonucleotide molecular scaffold for DNA.

FIG. 5B illustrates an example circular polyribonucleotide molecular scaffold with a sequence specific DNA binding motif. The circRNA can bind to the major groove of the DNA duplex to form parallel or antiparallel triplex structures based on the orientation of the third strand. Exemplary parallel triplex structures include TA U, CGG and CGC (DNA DNA RNA). Exemplary antiparallel triplex structures include TA A, TA-U and CG G (DNA DNA RNA).

FIG. 6 illustrates an example circular polyribonucleotide molecular scaffold for RNA.

FIG. 7A illustrates an example circular polyribonucleotide molecular scaffold for target RNAs to sequester and/or degrade target RNAs.

FIG. 7B illustrates an example circular polyribonucleotide molecular scaffold for RNAs and enzymes targeting the RNAs (e.g., decapping enzymes that induce degradation of the RNAs).

FIG. 7C illustrates an example circular polyribonucleotide molecular scaffold for RNA, DNA and protein (e.g., to drive target gene translation).

FIG. 8 shows circular RNA comprising an aptamer (circular aptamer) that binds to TO-1 biotin fluoresces in cells after delivery in the absence of a transfection reagent (carrier-independent delivery) compared to no detected fluorescence after delivery of a linear RNA comprising an aptamer that binds to TO-1 biotin or the buffer alone without an aptamer.

FIG. 9 shows a schematic of circular RNAs. The bottom left schematic shows a circular RNA comprising a C2 min aptamer sequence that binds the transferrin receptor. The bottom middle schematic shows a circular RNA comprising a 36a aptamer sequence that binds the transferrin receptor. The bottom right schematic shows a circular RNA comprising a non-binding sequence that does not bind the transferrin receptor. All three circular RNAs also comprise a sequence that binds to an AF488 labelled DNA oligonucleotide (annealing sequence).

FIG. 10 shows circular polyribonucleotides comprising an aptamer sequence (C2.min or 36a) that binds the transferrin receptor were internalized into cells that express the transferrin receptor based on fluorescence. The circular polyribonucleotides comprising the non-binding aptamer were not internalized into cells that express the transferrin receptor based on fluorescence.

FIG. 11 shows a schematic of a circular RNA hybridized to a single-stranded RNA oligonucleotide comprising an aptamer sequence. The single-stranded RNA oligonucleotide comprises an aptamer sequence and a sequence that binds to the circular polyribonucleotide (binding motif). The circular RNA comprises a sequence that binds to a binding sequence in the binding motif of the single-stranded RNA oligonucleotide. The bottom left schematic shows a single-stranded RNA oligonucleotide comprising a C2 min aptamer sequence that binds the transferrin receptor and a sequence that binds to the circular polyribonucleotide, which is bound to the circular polyribonucleotide. The bottom middle schematic shows a single-stranded RNA oligonucleotide comprising a 36a aptamer sequence that binds the transferrin receptor and a sequence that binds to the circular polyribonucleotide, which is bound to the circular polyribonucleotide. The bottom right schematic shows a single-stranded RNA oligonucleotide comprising an aptamer sequence that is non-binding for the transferrin receptor and a sequence that binds to the circular polyribonucleotide, which is bound to the circular polyribonucleotide.

FIG. 12 shows a schematic of a circular RNA hybridized to a single-stranded DNA oligonucleotide comprising an aptamer sequence. The single-stranded DNA oligonucleotide comprises an aptamer sequence and a sequence that binds to the circular polyribonucleotide (binding motif). The circular RNA comprises a sequence that binds to a binding sequence in the binding motif of the single-stranded DNA oligonucleotide. The bottom left schematic shows a single-stranded DNA oligonucleotide comprising a competitively binding aptamer sequence that binds the transferrin receptor and a sequence that binds to the circular polyribonucleotide, which is bound to the circular polyribonucleotide. The bottom middle schematic shows a single-stranded DNA oligonucleotide comprising a non-competitively binding aptamer sequence that binds the transferrin receptor and a sequence that binds to the circular polyribonucleotide, which is bound to the circular polyribonucleotide. The bottom right schematic shows a single-stranded DNA oligonucleotide comprising a aptamer sequence that is non-binding for the transferrin receptor and a sequence that binds to the circular polyribonucleotide, which is bound to the circular polyribonucleotide.

FIGS. 13A, 13B, and 13C show that the modified circular RNAs bind protein translation machinery in cells.

FIGS. 14A, 14B, and 14C show that modified circular RNAs have reduced binding to immune proteins as assessed by activation of immune related genes (MDA5, OAS, and IFN-beta expression) as compared to unmodified circular RNAs in cells.

FIG. 15 shows that hybrid modified circular RNAs have reduced immunogenicity as compared to unmodified circular RNAs as assessed by RIG-I, MDA5, IFN-beta, and OAS expression in cells.

FIG. 16 demonstrates that a circular RNA aptamer exhibits increased intracellular delivery and enhanced binding to a small molecule target compared to a linear aptamer.

FIG. 17 demonstrates a small molecule-circular RNA conjugate binds to a protein targeted by the small molecule.

FIG. 18 shows images from an electrophoretic mobility shift assay (EMSA) demonstrating that RNA with scrambled binding aptamer sequences did not show binding affinity to the p50 subunit of NF-kB, while both linear and circular RNAs with the NF-kB binding aptamer sequence bound to the p50 subunit with similar affinities.

FIG. 19 shows that treatment with circular RNA with the NF-kB binding aptamer sequence led to a decrease in cell viability of A549 cells as compared to its linear counterpart.

FIG. 20 shows co-treatment with linear RNA and doxorubicin (dox) decreased cell viability at day 2 and co-treatment with the circular aptamer and dox resulted in more cell death at both days 1 and 2 in the dox-resistant A549 lung cancer cell line.

FIG. 21 is a schematic showing an exemplary circular RNA that is delivered into cells and tags a target BRD4 protein in the cells for degradation by ubiquitin system.

FIG. 22 shows Western blot images and quantitative chart demonstrating that circular RNA containing thalidomide and JQ1 small molecules was able to degrade BRD4 in cells.

FIG. 23 shows aptamer fluorescence when bound to TO-1 biotin at different time points after delivery of the circular RNA (endless aptamer) or the linear RNA (linear aptamer) to HeLa cell cultures. The fluorescent images (top) show aptamer fluorescence when bound to TO-1 biotin at 6 hours, Day 1, and Day 10 after delivery of the circular RNA (endless aptamer) or the linear RNA (linear aptamer). The graphs (bottom) show the percentage of fluorescent cells in the HeLa cell cultures at 6 hours, Day 1, Day 3, Day 5, Day 7, Day 10, and Day 12 after delivery of the circular RNA (endless aptamer), the linear RNA (linear aptamer), or the TO-1 biotin only (control).

FIG. 24 shows HuR bound circular RNAs with a HuR RNA binding aptamer motif and the streptavidin pull-down yielded RNAs with the RNA binding aptamer motifs compared to a circular RNA with no binding aptamer motifs, a circular RNA with a HuR RNA binding aptamer motif, and a circular RNA with an RNA binding aptamer motif.

FIG. 25 shows HuR bound circular RNAs with the HuR DNA binding aptamer motif and the streptavidin pull-down yielded RNAs with the DNA binding aptamer motifs compared to a circular RNA with no binding aptamer motifs, a circular RNA with a HuR DNA binding aptamer motif, and a circular RNA with DNA.

FIG. 26 shows lower secreted protein expression from circular RNA without a HuR binding motif compared to a circular RNA with 1×HuR binding motif, 2×HuR binding motifs, and 3×HuR binding motifs.

### DETAILED DESCRIPTION

This invention relates generally to the delivery of compositions of circular polyribonucleotides.

The invention as disclosed herein includes a parenteral delivery system comprising a circular polyribonucleotide and a parenterally acceptable diluent, wherein the circular polyribonucleotide comprises a binding site, e.g., a sequence that binds a target. The parenteral delivery system can be a delivery system free of any carrier. The parenteral delivery system can further comprise a carrier.

The invention as disclosed herein includes a parenteral delivery system comprising a circular polyribonucleotide and a parenterally acceptable diluent, wherein the circular polyribonucleotide is a translation incompetent circular polyribonucleotide and comprises a sequence that binds a target. The parenteral delivery system can be a delivery system free of any carrier. The parenteral delivery system can further comprise a carrier.

The invention as disclosed herein includes a method of in vivo delivery of a circular polyribonucleotide comprising parenterally administering the circular polyribonucleotide to a subject, wherein the circular polyribonucleotide comprises a sequence that binds a target. In some embodiments, a method of in vivo delivery of a circular polyribonucleotide to a cell or tissue of a subject comprising parenterally administering to the cell or tissue the circular polyribonucleotide, wherein the circular polyribonucleotide comprises a sequence that binds a target. The circular polyribonucleotide can be in a composition. The composition can comprise a carrier. In some embodiments, the composition comprises a parenterally acceptable diluent and is free of any carrier. Parenteral administration can comprise intramuscular administration, intravenous administration, ophthalmic administration, or topical administration.

The invention as disclosed herein includes a method of in vivo delivery of a circular polyribonucleotide comprising parenterally administering the circular polyribonucleotide to a subject, wherein the circular polyribonucleotide is a translation incompetent circular polyribonucleotide and comprises a sequence that binds a target. In some embodiments, a method of in vivo delivery of a circular polyribonucleotide to a cell or tissue of a subject comprising parenterally administering to the cell or tissue the circular polyribonucleotide, wherein the circular polyribonucleotide is a translation incompetent circular polyribonucleotide and comprises a sequence that binds a target. The circular polyribonucleotide can be in a composition. The composition can comprise a carrier. In some embodiments, the composition comprises a parenterally acceptable diluent and is free of any carrier. Parenteral administration can comprise intramuscular administration, intravenous administration, ophthalmic administration, or topical administration.

The invention includes a parenteral nucleic acid delivery system as described herein for use as a medicament or a pharmaceutical.

The invention includes a parenteral nucleic acid delivery system as described for use in a method of treatment of a human or animal body by therapy.

The invention includes a parenteral nucleic acid delivery system as described herein in the manufacture of a medicament or a pharmaceutical.

The invention includes a parenteral nucleic acid delivery system as described herein in the manufacture of a medicament or a pharmaceutical for treating a human or animal body by therapy.

The circular polyribonucleotides administered in the methods of delivery as described herein can bind to a target. The circular polyribonucleotide can comprise a sequence that binds to (e.g., hybridizes) to a target. The circular polyribonucleotide can comprise an aptamer having a secondary structure that binds to a target. In some embodiments, the administered circular polyribonucleotides are completely modified circular polyribonucleotides. In some embodiments, the administered circular polyribonucleotides are hybrid modified circular polyribonucleotides. In other embodiments, the administered circular polyribonucleotides are unmodified circular polyribonucleotides. After administration to a cell or tissue of a subject or to a subject, the circular polyribonucleotides described herein can modulate cellular function or a cellular process, e.g., gene expression in the cell, tissue, or subject. The circular polyribonucleotide can lack a poly-A sequence, lack a replication element, lack a free 3′ end, or lack an RNA polymerase recognition motif, or any combination thereof. In some embodiments, the circular polyribonucleotide is a translation incompetent circular polyribonucleotide. In some embodiments, the circular polyribonucleotide further comprises an expression sequence. In some embodiments, the circular polyribonucleotide comprises a termination element or an IRES, or the combination thereof.

In some embodiments, a method of binding a target in a cell comprises providing a circular polyribonucleotide comprising an aptamer sequence with a secondary structure that binds the target; and delivering the circular polyribonucleotide to the cell, wherein the circular polyribonucleotide forms a complex with the target detectable at least 5 days after delivery. In some embodiments, a method of binding a target in a cell comprises providing a translation incompetent circular polyribonucleotide comprising an aptamer sequence with a secondary structure that binds the target; and delivering the translation incompetent circular polyribonucleotide to the cell, wherein the translation incompetent circular polyribonucleotide forms a complex with the target detectable at least 5 days after delivery.

In some embodiments, a composition comprises a circular polyribonucleotide comprises an aptamer sequence with a secondary structure that binds a target. In some aspects, a composition comprises a translation incompetent circular polyribonucleotide comprises an aptamer sequence with a secondary structure that binds a target.

In some aspects, a pharmaceutical composition comprises a translation incompetent circular polyribonucleotide comprising an aptamer sequence with a secondary structure that binds the target; and a pharmaceutically acceptable carrier or excipient.

In some embodiments, a cell comprises the circular polyribonucleotide as described herein. In some embodiments, a cell comprises the translation incompetent circular polyribonucleotide as described herein.

In some embodiments, a pharmaceutical composition comprises a circular polyribonucleotide comprising a binding site that binds a target, e.g., a RNA, DNA, protein, membrane of cell etc.; and a pharmaceutically acceptable carrier or excipient; wherein the target and the circular polyribonucleotide form a complex, and wherein the target is a not a microRNA. In some aspects, a pharmaceutical composition comprises a circular polyribonucleotide comprising: a first binding site that binds a first target, and a second binding site that binds a second target; and a pharmaceutically acceptable carrier or excipient; wherein the first binding site is different than the second binding site, and wherein the first target and the second target are both a microRNA.

In some embodiments, a pharmaceutical composition comprises a circular polyribonucleotide comprising a binding site that binds a target; and a pharmaceutically acceptable carrier or excipient; and wherein the target is not a microRNA. In some aspects, a pharmaceutical composition comprises a circular polyribonucleic acid comprising a binding site that binds a target, wherein the target comprises a ribonucleic acid (RNA)-binding motif, and a pharmaceutically acceptable carrier or excipient; and wherein the target is a microRNA. In some embodiments, a pharmaceutical composition comprises a circular polyribonucleotide comprising a binding site that binds a target; and a pharmaceutically acceptable carrier or excipient; wherein the circular polyribonucleotide is translation incompetent or translation defective, and wherein the target is not a microRNA. In some aspects, a pharmaceutical composition comprises a circular polyribonucleic acid comprising a binding site that binds a target, wherein the target comprises a ribonucleic acid (RNA)-binding motif, and a pharmaceutically acceptable carrier or excipient; wherein the circular polyribonucleotide is translation incompetent or translation defective, and wherein the target is a microRNA. In some embodiments, the binding site comprises an aptamer sequence having a secondary structure that binds the target.

### Parenteral Delivery System

A parenteral delivery system can be used for in vivo delivery of circular polyribonucleotides (circRNA) described herein.

In some embodiments, the parenteral delivery system comprises a circular polyribonucleotide and a parenterally acceptable diluent.

CircRNA are polyribonucleotides that form a continuous structure through covalent or non-covalent bonds. The circRNA can be any circular polyribonucleotide as described herein. In some embodiments, the circRNA comprises a sequence that binds a target. In some embodiments, the circRNA is a translation incompetent circular polyribonucleotide and comprises a sequence that binds a target. The sequence that binds a target can be an aptamer sequence that has a secondary structure that binds the target. The sequence that binds a target can hybridize to a target. Due to the circular structure, circRNA can have improved stability, increased half-life, reduced immunogenicity, and/or improved functionality (e.g., of a function described herein) compared to a corresponding linear RNA. In some embodiments, the circular RNA is detectable for at least 5 days after delivery of the circular RNA to a cell. In some embodiments, the circular RNA is detectable for at least 6 days, 7 days, 8 days, 9 days, 10 days, 11 days, 12 days, 13 days, 14 days, 15 days, or 16 days after delivery of the circular RNA to the cell. In some embodiments, the circular RNA is detectable for 5 days after delivery of the circular RNA to a cell. In some embodiments, the circular RNA is detectable for 6 days after delivery of the circular RNA to a cell. In some embodiments, the circular RNA is detectable for 7 days after delivery of the circular RNA to a cell. In some embodiments, the circular RNA is detectable for 8 days after delivery of the circular RNA to a cell. In some embodiments, the circular RNA is detectable for 9 days after delivery of the circular RNA to a cell. In some embodiments, the circular RNA is detectable for 10 days after delivery of the circular RNA to a cell. In some embodiments, the circular RNA is detectable for 11 days, In some embodiments, the circular RNA is detectable for 12 days after delivery of the circular RNA to a cell. In some embodiments, the circular RNA is detectable for 13 days after delivery of the circular RNA to a cell. In some embodiments, the circular RNA is detectable for 14 days after delivery of the circular RNA to a cell. In some embodiments, the circular RNA is detectable for 15 days after delivery of the circular RNA to a cell. In some embodiments, the circular RNA is detectable for 16 days after delivery of the circular RNA to the cell. The circRNA can be in a composition. In some embodiments, the composition is a pharmaceutical composition further comprising a pharmaceutically acceptable excipient.

A diluent (e.g., a parenterally acceptable diluent) is a vehicle comprising an inactive solvent in which a composition described herein (e.g., a composition comprising a circular polyribonucleotide) may be diluted or dissolved. A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, or powdered sugar.

In some embodiments, the parenteral delivery system further comprises a carrier. A carrier is a compound, composition, reagent, or molecule that facilitates the transport or delivery of a circular polyribonucleotide as described herein into a cell by a covalent modification of the circular polyribonucleotide, via a partially or completely encapsulating agent, or a combination thereof. Non-limiting examples of carriers include carbohydrate carriers (e.g., an anhydride-modified phytoglycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

A parenteral delivery system as disclosed herein can be used as a medicament or a pharmaceutical. A parenteral delivery system as disclosed herein can be used in a method of treatment of a human or animal body by therapy. A parenteral delivery system as disclosed herein can be used in the manufacture of a medicament or a pharmaceutical. A parenteral delivery system as disclosed herein can be used in the manufacture of a medicament or a pharmaceutical for treating a human or animal body by therapy.

### Pharmaceutical Compositions

The present invention includes the circular polyribonucleotide as disclosed herein of the parenteral delivery system in combination with one or more pharmaceutically acceptable excipients as a pharmaceutical composition. A pharmaceutically acceptable excipient can be a non-carrier excipient. A non-carrier excipient serves as a vehicle or medium for a composition, such as a circular polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. A pharmaceutically acceptable excipient can optionally comprise one or more active substances, e.g., therapeutically and/or prophylactically active substances. Pharmaceutical compositions of the present invention can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005, which is incorporated herein by reference.

Pharmaceutical compositions described herein can be used in therapeutic and veterinary. In some embodiments, pharmaceutical compositions (e.g., comprising a circular polyribonucleotide as described herein) provided herein are suitable for administration to a subject, wherein the subject is a non-human animal, for example, suitable for veterinary use. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, any animals, such as humans and/or other primates; mammals, including commercially relevant mammals, e.g., pet and live-stock animals, such as cattle, pigs, horses, sheep, goats, cats, dogs, mice, and/or rats; and/or birds, including commercially relevant birds such as parrots, poultry, chickens, ducks, geese, hens or roosters and/or turkeys; zoo animals, e.g., a feline; non-mammal animals, e.g., reptiles, fish, amphibians, etc.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

### Methods for In Vivo Delivery

The present invention includes methods of in vivo delivery of circular polyribonucleotides and compositions thereof. In some embodiments, a method of in vivo delivery of a circular polyribonucleotide comprises parenterally administering the circular polyribonucleotide or composition thereof to a subject, wherein the circular polyribonucleotide comprises a sequence that binds a target. In some embodiments, a method of in vivo delivery of a circular polyribonucleotide comprises parenterally administering the circular polyribonucleotide or composition thereof to a subject, wherein the circular polyribonucleotide is a translation incompetent circular polyribonucleotide and comprises a sequence that binds a target. In embodiments, the circular polyribonucleotide is in an amount effective to elicit a biological response in the subject. In some embodiments, the circular polyribonucleotide is in an amount effective to have a biological response in the subject.

In some embodiments, a method of in vivo delivery as described herein is a method of in vivo delivery of a circular polyribonucleotide or composition thereof to a cell or tissue of a subject comprising parenterally administering to the cell or tissue the circular polyribonucleotide or composition thereof, wherein the circular polyribonucleotide comprises a sequence that binds a target. In some embodiments, a method of in vivo delivery as described herein is a method of in vivo delivery of a circular polyribonucleotide or composition thereof to a cell or tissue of a subject comprising parenterally administering to the cell or tissue the circular polyribonucleotide or composition thereof, wherein the circular polyribonucleotide is a translation incompetent circular polyribonucleotide and comprises a sequence that binds a target.

In some embodiments, the administration of the circular polyribonucleotide or composition thereof is conducted using any delivery method described herein. In some embodiments, the parenteral administration of the circular polyribonucleotide or composition thereof is administered to the subject via intravenous injection. In some embodiments, the parenteral administration of the circular polyribonucleotide or composition thereof includes, but is not limited to, prenatal administration, neonatal administration, postnatal administration, oral, by injection (e.g., intravenous, intraarterial, intraperitoneal, intradermal, subcutaneous and intramuscular), by ophthalmic administration, and by intranasal administration. In some embodiments, the parenteral administration of the circular polyribonucleotide or composition thereof is prenatal administration. In some embodiments, the parenteral administration of the circular polyribonucleotide or composition thereof is neonatal administration. In some embodiments, the parenteral administration of the circular polyribonucleotide or composition thereof is postnatal administration. In some embodiments, the parenteral administration of the circular polyribonucleotide or composition thereof is oral. In some embodiments, the parenteral administration of the circular polyribonucleotide or composition thereof is by injection. In some embodiments, the parenteral administration of the circular polyribonucleotide or composition thereof is by intravenous injection. In some embodiments, the parenteral administration of the circular polyribonucleotide or composition thereof is by intraarterial injection. In some embodiments, the parenteral administration of the circular polyribonucleotide or composition thereof is by intraperitoneal injection. In some embodiments, the parenteral administration of the circular polyribonucleotide or composition thereof is by intradermal injection. In some embodiments, the parenteral administration of the circular polyribonucleotide or composition thereof is by subcutaneous injection. In some embodiments, the parenteral administration of the circular polyribonucleotide or composition thereof is by intramuscular injection. In some embodiments, the parenteral administration of the circular polyribonucleotide or composition thereof is by ophthalmic administration. In some embodiments, the parenteral administration of the circular polyribonucleotide or composition thereof is intranasal administration. In some embodiments, the compositions are parenterally administered and comprise a carrier. In some embodiments, the parenteral administration of the circular polyribonucleotide or composition thereof is prenatal administration and comprises a carrier. In some embodiments, the parenteral administration of the circular polyribonucleotide or composition thereof is neonatal administration and comprises a carrier. In some embodiments, the parenteral administration of the circular polyribonucleotide or composition thereof is postnatal administration and comprises a carrier. In some embodiments, the parenteral administration of the circular polyribonucleotide or composition thereof is oral and comprises a carrier. In some embodiments, the parenteral administration of the circular polyribonucleotide or composition thereof is by injection and comprises a carrier. In some embodiments, the parenteral administration of the circular polyribonucleotide or composition thereof is by intravenous injection and comprises a carrier. In some embodiments, the parenteral administration of the circular polyribonucleotide or composition thereof is by intraarterial injection and comprises a carrier. In some embodiments, the parenteral administration of the circular polyribonucleotide or composition thereof is by intraperitoneal injection and comprises a carrier. In some embodiments, the parenteral administration of the circular polyribonucleotide or composition thereof is by intradermal injection and comprises a carrier. In some embodiments, the parenteral administration of the circular polyribonucleotide or composition thereof is by subcutaneous injection and comprises a carrier. In some embodiments, the parenteral administration of the circular polyribonucleotide or composition thereof is by intramuscular injection and comprises a carrier. In some embodiments, the parenteral administration of the circular polyribonucleotide or composition thereof is by ophthalmic administration and comprises a carrier. In some embodiments, the parenteral administration of the circular polyribonucleotide or composition thereof is intranasal administration and comprises a carrier. In some embodiments, the compositions are parenterally administered and comprise a diluent. In some embodiments, the parenteral administration of the circular polyribonucleotide or composition thereof is prenatal administration and comprises a diluent. In some embodiments, the parenteral administration of the circular polyribonucleotide or composition thereof is neonatal administration and comprises a diluent. In some embodiments, the parenteral administration of the circular polyribonucleotide or composition thereof is postnatal administration and comprises a diluent. In some embodiments, the parenteral administration of the circular polyribonucleotide or composition thereof is oral and comprises a diluent. In some embodiments, the parenteral administration of the circular polyribonucleotide or composition thereof is by injection and comprises a diluent. In some embodiments, the parenteral administration of the circular polyribonucleotide or composition thereof is by intravenous injection and comprises a diluent. In some embodiments, the parenteral administration of the circular polyribonucleotide or composition thereof is by intraarterial injection and comprises a diluent. In some embodiments, the parenteral administration of the circular polyribonucleotide or composition thereof is by intraperitoneal injection and comprises a diluent. In some embodiments, the parenteral administration of the circular polyribonucleotide or composition thereof is by intradermal injection and comprises a diluent. In some embodiments, the parenteral administration of the circular polyribonucleotide or composition thereof is by subcutaneous injection and comprises a diluent. In some embodiments, the parenteral administration of the circular polyribonucleotide or composition thereof is by intramuscular injection and comprises a diluent. In some embodiments, the parenteral administration of the circular polyribonucleotide or composition thereof is by ophthalmic administration and comprises a diluent. In some embodiments, the parenteral administration of the circular polyribonucleotide or composition thereof is intranasal administration and comprises a diluent. In some embodiments, the compositions are parenterally administered and comprise a parenterally acceptable diluent. In some embodiments, the parenteral administration of the circular polyribonucleotide or composition thereof is prenatal administration and comprises a parenterally acceptable diluent. In some embodiments, the parenteral administration of the circular polyribonucleotide or composition thereof is neonatal administration and comprises a parenterally acceptable diluent. In some embodiments, the parenteral administration of the circular polyribonucleotide or composition thereof is postnatal administration and comprises a parenterally acceptable diluent. In some embodiments, the parenteral administration of the circular polyribonucleotide or composition thereof is oral and comprises a parenterally acceptable diluent. In some embodiments, the parenteral administration of the circular polyribonucleotide or composition thereof is by injection and comprises a parenterally acceptable diluent. In some embodiments, the parenteral administration of the circular polyribonucleotide or composition thereof is by intravenous injection and comprises a parenterally acceptable diluent. In some embodiments, the parenteral administration of the circular polyribonucleotide or composition thereof is by intraarterial injection and comprises a parenterally acceptable diluent. In some embodiments, the parenteral administration of the circular polyribonucleotide or composition thereof is by intraperitoneal injection and comprises a parenterally acceptable diluent. In some embodiments, the parenteral administration of the circular polyribonucleotide or composition thereof is by intradermal injection and comprises a parenterally acceptable diluent. In some embodiments, the parenteral administration of the circular polyribonucleotide or composition thereof is by subcutaneous injection and comprises a parenterally acceptable diluent. In some embodiments, the parenteral administration of the circular polyribonucleotide or composition thereof is by intramuscular injection and comprises a parenterally acceptable diluent. In some embodiments, the parenteral administration of the circular polyribonucleotide or composition thereof is by ophthalmic administration and comprises a parenterally acceptable diluent. In some embodiments, the parenteral administration of the circular polyribonucleotide or composition thereof is intranasal administration and comprises a parenterally acceptable diluent. In some embodiments, the compositions are parenterally administered and lack a carrier. In some embodiments, the parenteral administration of the circular polyribonucleotide or composition thereof is prenatal administration and lacks a carrier. In some embodiments, the parenteral administration of the circular polyribonucleotide or composition thereof is neonatal administration and lacks a carrier. In some embodiments, the parenteral administration of the circular polyribonucleotide or composition thereof is postnatal administration and lacks a carrier. In some embodiments, the parenteral administration of the circular polyribonucleotide or composition thereof is oral and lacks a carrier. In some embodiments, the parenteral administration of the circular polyribonucleotide or composition thereof is by injection and lacks a carrier. In some embodiments, the parenteral administration of the circular polyribonucleotide or composition thereof is by intravenous injection and lacks a carrier. In some embodiments, the parenteral administration of the circular polyribonucleotide or composition thereof is by intraarterial injection and lacks a carrier. In some embodiments, the parenteral administration of the circular polyribonucleotide or composition thereof is by intraperitoneal injection and lacks a carrier. In some embodiments, the parenteral administration of the circular polyribonucleotide or composition thereof is by intradermal injection and lacks a carrier. In some embodiments, the parenteral administration of the circular polyribonucleotide or composition thereof is by subcutaneous injection and lacks a carrier. In some embodiments, the parenteral administration of the circular polyribonucleotide or composition thereof is by intramuscular injection and lacks a carrier. In some embodiments, the parenteral administration of the circular polyribonucleotide or composition thereof is by ophthalmic administration and lacks a carrier. In some embodiments, the parenteral administration of the circular polyribonucleotide or composition thereof is intranasal administration and lacks a carrier.

In some embodiments, a use of a circular polyribonucleotide in the manufacture of a parenteral composition is for delivering to a cell or tissue of a subject, wherein the circular polyribonucleotide comprises a sequence that binds a target. In some embodiments, the parenteral composition is formulated for intravenous, intramuscular, ophthalmical or topical administration. In some embodiments, the parenteral composition is a pharmaceutical composition further comprising a pharmaceutically acceptable excipient. In some embodiments, the parenteral composition comprises a carrier. In some embodiments, the parenteral composition comprises a parenterally acceptable diluent and is free of any carrier. In some embodiments, the circular polyribonucleotide of the parenteral composition forms a complex with the target and the circular polyribonucleotide or the target is detectable at least 5 days after delivery. In some embodiments, the circular polyribonucleotide of the complex or the target of the complex is detectable at least 7, 8, 9, or 10 days after delivery. In some embodiments, the circular polyribonucleotide of the complex or the target of the complex is detectable 7 days after delivery. In some embodiments, the circular polyribonucleotide of the complex or the target of the complex is detectable 8 days after delivery. In some embodiments, the circular polyribonucleotide of the complex or the target of the complex is detectable 9 days after delivery. In some embodiments, the circular polyribonucleotide of the complex or the target of the complex is detectable 10 days after delivery. In some embodiments, the circular polyribonucleotide of the parenteral composition is present at least five days after delivery. In some embodiments, the circular polyribonucleotide the parenteral composition is present at least 6, 7, 8, 9, or 10 days after delivery. In some embodiments, the circular polyribonucleotide the parenteral composition is present 6 days after delivery. In some embodiments, the circular polyribonucleotide the parenteral composition is present 7 days after delivery. In some embodiments, the circular polyribonucleotide the parenteral composition is present 8 days after delivery. In some embodiments, the circular polyribonucleotide the parenteral composition is present 9 days after delivery. In some embodiments, the circular polyribonucleotide the parenteral composition is present 10 days after delivery. In some embodiments, the circular polyribonucleotide of the parenteral composition is an unmodified circular polyribonucleotide. In some embodiments, the circular polyribonucleotide of the parenteral composition has a quasi-double-stranded secondary structure. In some embodiments, the sequence is an aptamer sequence that has a secondary structure that binds the target. In some embodiments, the aptamer sequence further has a tertiary structure that binds the target. In some embodiments, the circular polyribonucleotide of the parenteral composition lacks a poly-A sequence, lacks a replication element, lacks a free 3′ end, or lacks an RNA polymerase recognition motif, or any combination thereof. In some embodiments, the circular polyribonucleotide of the parenteral composition lacks a poly-A sequence. In some embodiments, the circular polyribonucleotide of the parenteral composition lacks a replication element. In some embodiments, the circular polyribonucleotide of the parenteral composition lacks a free 3′ end. In some embodiments, the circular polyribonucleotide of the parenteral composition lacks an RNA polymerase recognition motif. In some embodiments, the circular polyribonucleotide of the parenteral composition is a translation incompetent circular polyribonucleotide. In some embodiments, the circular polyribonucleotide further comprises an expression sequence. In some embodiments, the circular polyribonucleotide of the parenteral composition comprises a termination element or an IRES, or the combination thereof.

In some embodiments, the cell is a eukaryotic cell. In some embodiments, the cell is a mammalian cell. In some embodiments, the cell is a human cell. In some embodiments, the cell is an animal cell. In some embodiments, the cell is an immune cell. In some embodiments, the cell is a livestock cell. In some embodiments, the tissue is a connective tissue. In some embodiments, the tissue is a muscle tissue. In some embodiments, the tissue is a nervous tissue. In some embodiments, the tissue is an epithelial tissue. In some embodiments, the subject is a mammal. In some embodiments, the subject is a human. In some embodiments, the subject is a pet. In some embodiments, the subject is a live-stock animal.

In some embodiments, the parenteral nucleic acid delivery system as disclosed herein (e.g., the nucleic acid compositions in the methods of delivery as described above) is used as a medicament or a pharmaceutical. A parenteral nucleic acid delivery system as disclosed herein can be used in a method of treatment of a human or animal body by therapy. A parenteral nucleic acid delivery system as disclosed herein can be used in the manufacture of a medicament or a pharmaceutical. A parenteral nucleic acid delivery system as disclosed herein can be used in the manufacture of a medicament or a pharmaceutical for treating a human or animal body by therapy.

### Delivery

The parenteral delivery system and methods of delivery as described herein comprise compositions of circular polyribonucleotides and methods of parenteral administration. A parenteral delivery system can comprise a circular polyribonucleotide and a parenterally acceptable diluent or excipient. In some embodiments, the delivery system comprises a carrier. In some embodiments, the delivery system is free of any carrier. In some embodiments, a composition, or pharmaceutical composition comprises the circular polyribonucleotide and parenterally acceptable diluent. In some embodiments, the composition or pharmaceutical composition further is free of any carrier. Methods as disclosed herein include a method of in vivo delivery of a circular polyribonucleotide as disclosed herein, composition as disclosed herein, or a pharmaceutical composition as disclosed herein comprising parenterally administering the circular polyribonucleotide, composition, or a pharmaceutical composition to the cell or tissue of a subject, or to a subject.

Pharmaceutical compositions as described herein can be formulated for example to include a pharmaceutical excipient or carrier. A pharmaceutical carrier may be a membrane, lipid bilayer, and/or a polymeric carrier, e.g., a liposome or particle such as a nanoparticle, e.g., a lipid nanoparticle, and delivered by known methods, such as via partial or full encapsulation of the circular polyribonucleotides, to a subject in need thereof (e.g., a human or non-human agricultural or domestic animal, e.g., cattle, dog, cat, horse, poultry).

Such methods include, but are not limited to, transfection (e.g., lipid-mediated, cationic polymers, calcium phosphate, dendrimers); viral delivery (e.g., lentivirus, retrovirus, adenovirus, AAV), fugene, protoplast fusion, exosome-mediated transfer, lipid nanoparticle-mediated transfer, and any combination thereof. Cationic lipid-mediated delivery of proteins enables efficient protein-based genome editing in vitro and in vivo. Nat Biotechnol. 2014 Oct. 30; 33(1):73-80. Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al.

Additional methods of delivery include electroporation (e.g., using a flow electroporation device) or other methods of membrane disruption (e.g., nucleofection), microinjection, microprojectile bombardment (“gene gun”), direct sonic loading, cell squeezing, optical transfection, impalefection, magnetofection, and any combination thereof. A flow electroporation device, for example, comprises a chamber for containing a suspension of cells to be electorporated, such as the cells (e.g., isolated cells) as described herein, the chamber being at least partially defined by oppositely chargeable electrodes, wherein the thermal resistance of the chamber is less than approximately 110° C. per Watt.

In some embodiments, the circular polyribonucleotide, composition, or a pharmaceutical composition may be delivered as a naked delivery formulation. A naked delivery formulation delivers a circular polyribonucleotide or protein to a cell without the aid of a carrier and without covalent modification or partial or complete encapsulation of the circular polyribonucleotide.

A naked delivery formulation is a formulation that is free from a carrier and wherein the circular polyribonucleotide is without a covalent modification that binds a moiety that aids in delivery to a cell or without partial or complete encapsulation of the circular polyribonucleotide. In some embodiments, a circular polyribonucleotide without covalent modification bound to a moiety that aids in delivery to a cell is not covalently bound to a protein, small molecule, a particle, a polymer, or a biopolymer that aids in delivery to a cell. A circular polyribonucleotide without a covalent modification that binds a moiety that aids in delivery to a cell does not, for example, contain a modified phosphate group, such as phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation may be free of any or all of: transfection reagents, cationic carriers, carbohydrate carriers, nanoparticle carriers, or protein carriers. For example, a naked delivery formulation may be free from phtoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B—[N—(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

A naked delivery formulation may comprise a non-carrier excipient. In some embodiments, a non-carrier excipient may comprise an inactive ingredient. In some embodiments, a non-carrier excipient may comprise a buffer, for example PBS. In some embodiments, a non-carrier excipient may be a solvent, a non-aqueous solvent, a diluent (e.g., a parenterally acceptable diluent), a suspension aid, a surface active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation may comprise a diluent (e.g., a parenterally acceptable diluent). A diluent may be a liquid diluent or a solid diluent. In some embodiments, a diluent may be an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

The invention is further directed to a host or host cell comprising the circular polyribonucleotide, composition, or a pharmaceutical composition as described herein. In some embodiments, vertebrate, mammal (e.g., human), or other organism or cell.

In some embodiments, the circular polyribonucleotide, composition, or a pharmaceutical composition is non-immunogenic in the host. In some embodiments, the circular polyribonucleotide, composition, or a pharmaceutical composition has a decreased or fails to produce a response by the host's immune system as compared to the response triggered by a reference compound, e.g., a linear polynucleotide corresponding to the described circular polyribonucleotide or a circular polyribonucleotide lacking an encryptogen. Some immune responses include, but are not limited to, humoral immune responses (e.g., production of antigen-specific antibodies) and cell-mediated immune responses (e.g., lymphocyte proliferation).

In some embodiments, a host or a host cell is contacted with (e.g., delivered to or administered to) the circular polyribonucleotide, composition, or a pharmaceutical composition. In some embodiments, the host is a mammal, such as a human. The amount of the circular polyribonucleotide, expression product, or both in the host can be measured at any time after administration. In certain embodiments, a time course of host growth in a culture is determined. If the growth is increased or reduced in the presence of the circular polyribonucleotide or protein, or expression product or both is identified as being effective in increasing or reducing the growth of the host.

### Methods of Delivery

A method of delivering a circular polyribonucleotide as described herein or a composition thereof as described herein to a cell, tissue or subject, comprises parenterally administering the circular polyribonucleotide or a composition thereof as described herein to the cell or tissue of a subject, or to a subject.

In some embodiments, the method of delivering is an in vivo method. For example, a method of delivering a circular polyribonucleotide as described herein comprises parenterally administering to a subject in need thereof, the circular polyribonucleotide, composition, or pharmaceutical composition as described herein to the subject in need thereof. In some embodiments, the circular polyribonucleotide is an amount effective to have a biological effect on the cell or tissue in the subject. In some embodiments, the pharmaceutical composition as described herein comprises a carrier. In some embodiments the pharmaceutical composition as described herein comprises a diluent and is free of any carrier. In some embodiments, parenteral administration is intravenously, intramuscularly, ophthalmically, or topically. In some embodiments, parenteral administration is intravenously. In some embodiments, parenteral administration intramuscularly. In some embodiments, parenteral administration ophthalmically. In some embodiments, parenteral administration topically.

In some embodiments the circular polyribonucleotide, a composition thereof, or pharmaceutical composition thereof is administered parenterally. In some embodiments, the circular polyribonucleotide, composition thereof, or pharmaceutical composition thereof is administered orally. In some embodiments, the circular polyribonucleotide, composition thereof, or pharmaceutical composition thereof is administered nasally. In some embodiments, the circular polyribonucleotide, composition thereof, or pharmaceutical composition thereof is administered by inhalation. In some embodiments, the circular polyribonucleotide, composition thereof, or pharmaceutical composition thereof is administered topically. In some embodiments, the circular polyribonucleotide, composition thereof, or pharmaceutical composition thereof is administered ophthalmically. In some embodiments the circular polyribonucleotide, composition thereof, or pharmaceutical composition thereof is administered rectally. In some embodiments the circular polyribonucleotide, composition thereof, or pharmaceutical composition thereof is administered by injection. The administration can be systemic administration or local administration. In some embodiments, the circular polyribonucleotide, composition thereof, or pharmaceutical composition thereof is administered intravenously, intraarterially, intraperotoneally, intradermally, intracranially, intrathecally, intralymphaticly, subcutaneously, or intramuscularly. In some embodiments, the circular polyribonucleotide, composition thereof, or pharmaceutical composition thereof is administered via intraocular administration, intracochlear (inner ear) administration, or intratracheal administration. In some embodiments, the circular polyribonucleotide, composition thereof, or pharmaceutical composition thereof is administered intravenously. In some embodiments, the circular polyribonucleotide, composition thereof, or pharmaceutical composition thereof is administered intraarterially. In some embodiments, the circular polyribonucleotide, composition thereof, or pharmaceutical composition thereof is administered intraperotoneally. In some embodiments, the circular polyribonucleotide, composition thereof, or pharmaceutical composition thereof is administered intradermally. In some embodiments, the circular polyribonucleotide, composition thereof, or pharmaceutical composition thereof is administered intracranially. In some embodiments, the circular polyribonucleotide, composition thereof, or pharmaceutical composition thereof is administered intrathecally. In some embodiments, the circular polyribonucleotide, composition thereof, or pharmaceutical composition thereof is administered intralymphaticly. In some embodiments, the circular polyribonucleotide, composition thereof, or pharmaceutical composition thereof is administered subcutaneously. In some embodiments, the circular polyribonucleotide, composition thereof, or pharmaceutical composition thereof is administered. In some embodiments, the circular polyribonucleotide, composition thereof, or pharmaceutical composition thereof is administered intramuscularly. In some embodiments, the circular polyribonucleotide, composition thereof, or pharmaceutical composition thereof is administered via intraocular administration. In some embodiments, the circular polyribonucleotide, composition thereof, or pharmaceutical composition thereof is administered via intracochlear (inner ear) administration. In some embodiments, the circular polyribonucleotide, composition thereof, or pharmaceutical composition thereof is administered via intratracheal administration. In some embodiments, any of the methods of delivery as described herein are performed with a carrier. In some embodiments, the circular polyribonucleotide, composition thereof, or pharmaceutical composition thereof is administered intravenously with a carrier. In some embodiments, the circular polyribonucleotide, composition thereof, or pharmaceutical composition thereof is administered intraarterially with a carrier. In some embodiments, the circular polyribonucleotide, composition thereof, or pharmaceutical composition thereof is administered intraperotoneally with a carrier. In some embodiments, the circular polyribonucleotide, composition thereof, or pharmaceutical composition thereof is administered intradermally with a carrier. In some embodiments, the circular polyribonucleotide, composition thereof, or pharmaceutical composition thereof is administered intracranially with a carrier. In some embodiments, the circular polyribonucleotide, composition thereof, or pharmaceutical composition thereof is administered intrathecally with a carrier. In some embodiments, the circular polyribonucleotide, composition thereof, or pharmaceutical composition thereof is administered intralymphaticly with a carrier. In some embodiments, the circular polyribonucleotide, composition thereof, or pharmaceutical composition thereof is administered subcutaneously with a carrier. In some embodiments, the circular polyribonucleotide, composition thereof, or pharmaceutical composition thereof is administered with a carrier. In some embodiments, the circular polyribonucleotide, composition thereof, or pharmaceutical composition thereof is administered intramuscularly with a carrier. In some embodiments, the circular polyribonucleotide, composition thereof, or pharmaceutical composition thereof is administered via intraocular administration with a carrier. In some embodiments, the circular polyribonucleotide, composition thereof, or pharmaceutical composition thereof is administered via intracochlear (inner ear) administration with a carrier. In some embodiments, the circular polyribonucleotide, composition thereof, or pharmaceutical composition thereof is administered via intratracheal administration with a carrier. In some embodiments, any methods of delivery as described herein are performed without the aid of a carrier in a naked delivery formulation. In some embodiments, any of the methods of delivery as described herein are performed without the aid of a carrier in a naked delivery formulation. In some embodiments, the circular polyribonucleotide, composition thereof, or pharmaceutical composition thereof is administered intravenously without the aid of a carrier in a naked delivery formulation. In some embodiments, the circular polyribonucleotide, composition thereof, or pharmaceutical composition thereof is administered intraarterially without the aid of a carrier in a naked delivery formulation. In some embodiments, the circular polyribonucleotide, composition thereof, or pharmaceutical composition thereof is administered intraperotoneally without the aid of a carrier in a naked delivery formulation. In some embodiments, the circular polyribonucleotide, composition thereof, or pharmaceutical composition thereof is administered intradermally without the aid of a carrier in a naked delivery formulation. In some embodiments, the circular polyribonucleotide, composition thereof, or pharmaceutical composition thereof is administered intracranially without the aid of a carrier in a naked delivery formulation. In some embodiments, the circular polyribonucleotide, composition thereof, or pharmaceutical composition thereof is administered intrathecally without the aid of a carrier in a naked delivery formulation. In some embodiments, the circular polyribonucleotide, composition thereof, or pharmaceutical composition thereof is administered intralymphaticly without the aid of a carrier in a naked delivery formulation. In some embodiments, the circular polyribonucleotide, composition thereof, or pharmaceutical composition thereof is administered subcutaneously without the aid of a carrier in a naked delivery formulation. In some embodiments, the circular polyribonucleotide, composition thereof, or pharmaceutical composition thereof is administered without the aid of a carrier in a naked delivery formulation. In some embodiments, the circular polyribonucleotide, composition thereof, or pharmaceutical composition thereof is administered intramuscularly without the aid of a carrier in a naked delivery formulation. In some embodiments, the circular polyribonucleotide, composition thereof, or pharmaceutical composition thereof is administered via intraocular administration without the aid of a carrier in a naked delivery formulation. In some embodiments, the circular polyribonucleotide, composition thereof, or pharmaceutical composition thereof is administered via intracochlear (inner ear) administration without the aid of a carrier in a naked delivery formulation. In some embodiments, the circular polyribonucleotide, composition thereof, or pharmaceutical composition thereof is administered via intratracheal administration without the aid of a cater in a naked delivery formulation.

### Cell and Vesicle-Based Carriers

The circular polyribonucleotide, composition, or pharmaceutical composition as described herein can be administered to a cell in a vesicle or other membrane-based carrier.

In embodiments, the circular polyribonucleotide, composition thereof, or pharmaceutical composition thereof is administered in or via a cell, vesicle or other membrane-based carrier. In one embodiment the circular polyribonucleotide, composition thereof, or pharmaceutical composition thereof can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for a circular polyribonucleotide molecule or the pharmaceutical composition thereof as described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Additional non-limiting examples of carriers include carbohydrate carriers (e.g., an anhydride-modified phytoglycogen or glycogen-type material), protein carriers (e.g., a protein covalently linked to the circular polyribonucleotide), or cationic carriers (e.g., a cationic lipopolymer or transfection reagent). Non-limiting examples of carbohydrate carriers include phtoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B—[N—(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high-density lipoprotein (HDL), or globulin.

Exosomes can also be used as drug delivery vehicles for a circular polyribonucleotide molecule or a pharmaceutical composition thereof described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a circular polyribonucleotide molecule or a pharmaceutical composition thereof described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the circular polyribonucleotide, composition thereof, or pharmaceutical composition thereof described herein.

Virosomes and virus-like particles (VLPs) can also be used as carriers to the circular polyribonucleotide, composition thereof, or pharmaceutical composition thereof described herein to targeted cells.

Plant nanovesicles and plant messenger packs (PMPs), e.g., as described in International Patent Publication Nos. WO2011097480, WO2013070324, WO2017004526, or WO2020041784 can also be used as carriers to deliver the circular RNA as described herein.

Microbubbles can also be used as carriers to deliver a circular polyribonucleotide molecule described herein. Microbubbles can also be used as carriers to deliver a linear polyribonucleotide described herein. See, e.g., U.S. Pat. No. 7,115,583; Beeri, R. et al., Circulation. 2002 Oct. 1; 106(14):1756-1759; Bez, M. et al., Nat Protoc. 2019 April; 14(4): 1015-1026; Hemot, S. et al., Adv Drug Deliv Rev. 2008 Jun. 30; 60(10): 1153-1166; Rychak, J. J. et al., Adv Drug Deliv Rev. 2014 June; 72: 82-93. In some embodiments, microbubbles are albumin-coated perfluorocarbon microbubbles.

Silk fibroin can also be used as a carrier to deliver a circular polyribonucleotide molecule described herein. See, e.g., Boopathy, A. V. et al., PNAS. 116.33 (2019): 16473-1678; and He, H. et al., ACS Biomater. Sci. Eng. 4.5(2018): 1708-1715.

### Circular Polyribonucleotides

Circular polyribonucleotides (circRNA) described herein are polyribonucleotides that form a continuous structure through covalent or non-covalent bonds.

The present invention described herein includes compositions comprising synthetic circRNA and methods of their use. Due to the circular structure, circRNA can have improved stability, increased half-life, reduced immunogenicity, and/or improved functionality (e.g., of a function described herein) compared to a corresponding linear RNA. In some embodiments, the circular RNA is detectable for at least 5 days after delivery of the circular RNA to a cell. In some embodiments, the circular RNA is detectable for at least 6 days, 7 days, 8 days, 9 days, 10 days, 11 days, 12 days, 13 days, 14 days, 15 days, or 16 days after delivery of the circular RNA to the cell. In some embodiments, the circular RNA is detectable for 6 days after delivery of the circular RNA to the cell. In some embodiments, the circular RNA is detectable for 7 days after delivery of the circular RNA to the cell. In some embodiments, the circular RNA is detectable for 8 days after delivery of the circular RNA to the cell. In some embodiments, the circular RNA is detectable for 9 days after delivery of the circular RNA to the cell. In some embodiments, the circular RNA is detectable for 10 days after delivery of the circular RNA to the cell. In some embodiments, the circular RNA is detectable for 11 days after delivery of the circular RNA to the cell. In some embodiments, the circular RNA is detectable for 12 days after delivery of the circular RNA to the cell. In some embodiments, the circular RNA is detectable for 13 days after delivery of the circular RNA to the cell. In some embodiments, the circular RNA is detectable for 14 days after delivery of the circular RNA to the cell. In some embodiments, the circular RNA is detectable for 15 days after delivery of the circular RNA to the cell. In some embodiments, the circular RNA is detectable for 16 days after delivery of the circular RNA to the cell. The circular RNA can be detected using any technique known in the art.

In some embodiments, circRNA binds one or more targets. In some embodiments, a circRNA is a circular aptamer. In one embodiment, a circRNA comprises one or more binding sites that bind to one or more targets. In one embodiment, the circ RNA comprises an aptamer sequence. In one embodiment, circRNA binds both a DNA target and a protein target and e.g., mediates transcription. In another embodiment, circRNA brings together a protein complex and e.g., mediates post-translational modifications or signal transduction. In another embodiment, circRNA binds two or more different targets, such as proteins, and e.g., shuttles these proteins to the cytoplasm, or mediates degradation of one or more of the targets.

In some embodiments, circRNA binds at least one of DNA, RNA, and proteins and thereby regulates cellular processes (e.g., alter protein expression, modulate gene expression, modulate cell signaling, etc.). In some embodiments, synthetic circRNA includes binding sites for interaction with a target or at least one moiety, e.g., a binding moiety, of DNA, RNA or proteins of choice to thereby compete in binding with the endogenous counterpart.

In some embodiments, the circular RNA forms a complex that regulates the cellular process (e.g., alter protein expression, modulate gene expression, modulate cell signaling, etc.). In some embodiments, the circular RNA sensitizes a cell to a cytotoxic agent (e.g., a chemotherapeutic agent) by binding to a target (e.g., a transcription factor), which results in reduce cell viability. For example, sensitizing the cell to the cytoxic agent results in decreased cell viability after the delivery of the cytotoxic agent and the circular RNA. In some embodiments, the decreased cell viability is decreased by at least 10%, 20%, 30%, 40%, 50%, 60%, 70%, 80%, or 90%, or any percentage therein.

In some embodiments, the complex is detectable for at least 5 days after delivery of the circular RNA to cell. In some embodiments, the complex is detectable for at least 6 days, 7 days, 8 days, 9 days, 10 days, 11 days, 12 days, 13 days, 14 days, 15 days, or 16 days after delivery of the circular RNA to the cell. In some embodiments, the complex is detectable for 6 days after delivery of the circular RNA to the cell. In some embodiments, the complex is detectable for 7 days after delivery of the circular RNA to the cell. In some embodiments, the complex is detectable for 8 days after delivery of the circular RNA to the cell. In some embodiments, the complex is detectable for 9 days after delivery of the circular RNA to the cell. In some embodiments, the complex is detectable for 10 days after delivery of the circular RNA to the cell. In some embodiments, the complex is detectable for 11 days after delivery of the circular RNA to the cell. In some embodiments, the complex is detectable for 12 days after delivery of the circular RNA to the cell. In some embodiments, the complex is detectable for 13 days after delivery of the circular RNA to the cell. In some embodiments, the complex is detectable for 14 days after delivery of the circular RNA to the cell. In some embodiments, the complex is detectable for 15 days after delivery of the circular RNA to the cell. In some embodiments, the complex is detectable for 16 days after delivery of the circular RNA to the cell.

In one embodiment, synthetic circRNA binds and/or sequesters miRNAs. In another embodiment, synthetic circRNA binds and/or sequesters proteins. In another embodiment, synthetic circRNA binds and/or sequesters mRNA. In another embodiment, synthetic circRNA binds and/or sequesters ribosomes. In another embodiment, synthetic circRNA binds and/or sequesters circRNA. In another embodiment, synthetic circRNA binds and/or sequesters long-noncoding RNA (lncRNA) or any other non-coding RNA, e.g., miRNA, tRNA, rRNA, snoRNA, ncRNA, siRNA, long-noncoding RNA, shRNA. Besides binding and/or sequestration sites, the circRNA may include a degradation element, which will result in degradation of the bound and/or sequestered RNA and/or protein.

In one embodiment, a circRNA comprises a lncRNA or a sequence of a lncRNA, e.g., a circRNA comprises a sequence of a naturally occurring, non-circular lncRNA or a fragment thereof. In one embodiment, a lncRNA or a sequence of a lncRNA is circularized, with or without a spacer sequence, to form a synthetic circRNA.

In one embodiment, a circRNA has ribozyme activity. In one embodiment, a circRNA can be used to act as a ribozyme and cleave pathogenic or endogenous RNA, DNA, small molecules or protein. In one embodiment, a circRNA has enzymatic activity. In one embodiment, synthetic circRNA is able to specifically recognize and cleave RNA (e.g., viral RNA). In another embodiment circRNA is able to specifically recognize and cleave proteins. In another embodiment circRNA is able to specifically recognize and degrade small molecules.

In one embodiment, a circRNA is an immolating or self-cleaving or cleavable circRNA. In one embodiment, a circRNA can be used to deliver RNA, e.g., miRNA, tRNA, rRNA, snoRNA, ncRNA, siRNA, long-noncoding RNA, shRNA. In one embodiment, synthetic circRNA is made up of microRNAs separated by (1) self-cleavable elements (e.g., hammerhead, splicing element), (2) cleavage recruitment sites (e.g., ADAR), (3) a degradable linker (e.g., glycerol), (4) a chemical linker, and/or (5) a spacer sequence. In another embodiment, synthetic circRNA is made up of siRNAs separated by (1) self-cleavable elements (e.g., hammerhead, splicing element), (2) cleavage recruitment sites (e.g., ADAR), (3) a degradable linker (e.g., glycerol), (4), chemical linker, and/or (5) a spacer sequence.

In one embodiment, a circRNA is a transcriptionally/replication competent circRNA. This circRNA can encode any type of RNA. In one embodiment, a synthetic circRNA has an anti-sense miRNA and a transcriptional element. In one embodiment, after transcription, linear functional miRNAs are generated from a circRNA. In one embodiment, a circRNA is a translation incompetent circular polyribonucleotide.

In one embodiment, a circRNA has one or more of the above attributes in combination with a translating element.

In some embodiments, a circRNA comprises at least one modified nucleotide. In some embodiments, a circRNA comprises at least 10%, 20%, 30%, 40%, 50%, 60%, 70%, or 80% modified nucleotides. In some embodiments, a circRNA comprises substantially all (e.g., greater than 80%, 85%, 90%, 95%, 97%, 98%, or 99%, or about 100%) modified nucleotides. In some embodiments, a circRNA comprises modified nucleotides and a portion of unmodified contiguous nucleotides, which can be referred to as a hybrid modified circRNA. A portion of unmodified contiguous nucleotides can be an unmodified binding site configured to bind a protein, DNA, RNA, or a cell target in a hybrid modified circRNA. A portion of unmodified contiguous nucleotides can be an unmodified IRES in a hybrid modified circRNA. In other embodiments, a circRNA lacks modified nucleotides, which can be referred to as an unmodified circRNA.

In some embodiments, the circular polyribonucleotide comprises one or more of the elements as described herein in addition to comprising a binding site (e.g., a sequence for binding to a target). In some embodiments, the circular polyribonucleotide lacks a poly-A tail. In some embodiments, the circular polyribonucleotide lacks a replication element. In some embodiments, the circular polyribonucleotide lacks an IRES. In some embodiments, the circular polyribonucleotide lacks a cap. In some embodiments, the circular polyribonucleotide comprises any feature or any combination of features as disclosed in WO2019/118919 in addition to a binding site, which is hereby incorporated by reference in its entirety.

### Targets

A circRNA can comprise at least one binding site for a target, e.g., for a binding moiety of a target. A circRNA can comprise at least one aptamer sequence that binds to a target. In some embodiments, the circRNA comprises one or more binding sites for one or more targets. Targets include, but are not limited to, nucleic acids (e.g., RNAs, DNAs, RNA-DNA hybrids), small molecules (e.g., drugs, fluorophores, metabolites), aptamers, polypeptides, proteins, lipids, carbohydrates, antibodies, viruses, virus particles, membranes, multi-component complexes, organelles, cells, other cellular moieties, any fragments thereof, and any combination thereof. (See, e.g., Fredriksson et al., (2002) Nat Biotech 20:473-77; Gullberg et al., (2004) PNAS, 101:8420-24). For example, a target is a single-stranded RNA, a double-stranded RNA, a single-stranded DNA, a double-stranded DNA, a DNA or RNA comprising one or more double stranded regions and one or more single stranded regions, an RNA-DNA hybrid, a small molecule, an aptamer, a polypeptide, a protein, a lipid, a carbohydrate, an antibody, an antibody fragment, a mixture of antibodies, a virus particle, a membrane, a multi-component complex, a cell, a cellular moiety, any fragment thereof, or any combination thereof.

In some embodiments, a target is a polypeptide, a protein, or any fragment thereof. For example, a target can be a purified polypeptide, an isolated polypeptide, a fusion tagged polypeptide, a polypeptide attached to or spanning the membrane of a cell or a virus or virion, a cytoplasmic protein, an intracellular protein, an extracellular protein, a kinase, a tyrosine kinase, a serine/threonine kinase, a phosphatase, an aromatase, a phosphodiesterase, a cyclase, a helicase, a protease, an oxidoreductase, a reductase, a transferase, a hydrolase, a lyase, an isomerase, a glycosylase, a extracellular matrix protein, a ligase, a ubiquitin ligase, any ligase that affects post-translational modification, an ion transporter, a channel, a pore, an apoptotic protein, a cell adhesion protein, a pathogenic protein, an aberrantly expressed protein, a transcription factor, a transcription regulator, a translation protein, an epigenetic factor, an epigenetic regulator, a chromatin regulator, a chaperone, a secreted protein, a ligand, a hormone, a cytokine, a chemokine, a nuclear protein, a receptor, a transmembrane receptor, a receptor tyrosine kinase, a G-protein coupled receptor, a growth factor receptor, a nuclear receptor, a hormone receptor, a signal transducer, an antibody, a membrane protein, an integral membrane protein, a peripheral membrane protein, a cell wall protein, a globular protein, a fibrous protein, a glycoprotein, a lipoprotein, a chromosomal protein, a proto-oncogene, an oncogene, a tumor-suppressor gene, any fragment thereof, or any combination thereof. In some embodiments, a target is a heterologous polypeptide. In some embodiments, a target is a protein overexpressed in a cell using molecular techniques, such as transfection. In some embodiments, a target is a recombinant polypeptide. For example, a target is in a sample produced from bacterial (e.g., E. coli), yeast, animal (e.g., a pet), mammalian (e.g., human, livestock), or insect cells (e.g., proteins overexpressed by the organisms). In some embodiments, a target is a polypeptide with a mutation, insertion, deletion, or polymorphism. In some embodiments, a target is a polypeptide naturally expressed by a cell (e.g., a healthy cell or a cell associated with a disease or condition). In some embodiments, a target is an antigen, such as a polypeptide used to immunize an organism or to generate an immune response in an organism, such as for antibody production.

In some embodiments, a target is an antibody. An antibody can specifically bind to a particular spatial and polar organization of another molecule. An antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. A naturally occurring antibody can be a protein comprising at least two heavy (H) chains and two light (L) chains inter-connected by disulfide bonds. Each heavy chain can be comprised of a heavy chain variable region (V_{H}) and a heavy chain constant region. The heavy chain constant region can comprise three domains, C_{H1}, C_{H2}, and C_{H3}. Each light chain can comprise a light chain variable region (V_{L}) and a light chain constant region. The light chain constant region can comprise one domain, C_{L}. The V_{H} and V_{L} regions can be further subdivided into regions of hypervariability, termed complementary determining regions (CDR), interspersed with regions that are more conserved, termed framework regions (FR). Each V_{H} and V_{L} can be composed of three CDRs and four FRs arranged from amino-terminus to carboxy-terminus in the following order: FR₁, CDR₁, FR₂, CDR₂, FR₃, CDR₃, and FR4. The constant regions of the antibodies may mediate the binding of the immunoglobulin to host tissues or factors, including various cells of the immune system (e.g., effector cells) and the first component (C1 q) of the classical complement system. The antibodies can be of any isotype (e.g., IgG, IgE, IgM, IgD, IgA and IgY), class (e.g., IgG₁, IgG₂, IgG₃, IgG₄, IgA₁ and IgA₂), subclass or modified version thereof. Antibodies may include a complete immunoglobulin or fragments thereof. An antibody fragment can refer to one or more fragments of an antibody that retain the ability to specifically bind to a binding moiety, such as an antigen. In addition, aggregates, polymers, and conjugates of immunoglobulins or their fragments are also included so long as binding affinity for a particular molecule is maintained. Examples of antibody fragments include a Fab fragment, a monovalent fragment consisting of the V_{L}, V_{H}, C_{L} and C_{H1} domains; a F(ab)₂ fragment, a bivalent fragment comprising two Fab fragments linked by a disulfide bridge at the hinge region; an Fd fragment consisting of the V_{H} and C_{H1} domains; an Fv fragment consisting of the V_{L} and V_{H} domains of a single arm of an antibody; a single domain antibody (dAb) fragment (Ward et al., (1989) Nature 341:544-46), which consists of a V_{H} domain; and an isolated CDR and a single chain Fragment (scFv) in which the V_{L} and V_{H} regions pair to form monovalent molecules (known as single chain Fv (scFv); See, e.g., Bird et al., (1988) Science 242:423-26; and Huston et al., (1988) PNAS 85:5879-83). Thus, antibody fragments include Fab, F(ab)₂, scFv, Fv, dAb, and the like. Although the two domains V_{L} and V_{H} are coded for by separate genes, they can be joined, using recombinant methods, by an artificial peptide linker that enables them to be made as a single protein chain. Such single chain antibodies include one or more antigen binding moieties. An antibody can be a polyvalent antibody, for example, bivalent, trivalent, tetravalent, pentavalent, hexavalanet, heptavalent, or octavalent antibodies. An antibody can be a multi-specific antibody. For example, bispecific, trispecific, tetraspecific, pentaspecific, hexaspecific, heptaspecific, or octaspecific antibodies can be generated, e.g., by recombinantly joining a combination of any two or more antigen binding agents (e.g., Fab, F(ab)₂, scFv, Fv, IgG). Multi-specific antibodies can be used to bring two or more targets into close proximity, e.g., degradation machinery and a target substrate to degrade, or a ubiquitin ligase and a substrate to ubiquitinate. These antibody fragments can be obtained using conventional techniques known to those of skill in the art, and the fragments can be screened for utility in the same manner as are intact antibodies. Antibodies can be human, humanized, chimeric, isolated, dog, cat, donkey, sheep, any plant, animal, or mammal.

In some embodiments, a target is a polymeric form of ribonucleotides and/or deoxyribonucleotides (adenine, guanine, thymine, or cytosine), such as DNA or RNA (e.g., mRNA). DNA includes double-stranded DNA found in linear DNA molecules (e.g., restriction fragments), viruses, plasmids, and chromosomes. In some embodiments, a polynucleotide target is single-stranded, double stranded, small interfering RNA (siRNA), messenger RNA (mRNA), transfer RNA (tRNA), a chromosome, a gene, a noncoding genomic sequence, genomic DNA (e.g., fragmented genomic DNA), a purified polynucleotide, an isolated polynucleotide, a hybridized polynucleotide, a transcription factor binding site, mitochondrial DNA, ribosomal RNA, a eukaryotic polynucleotide, a prokaryotic polynucleotide, a synthesized polynucleotide, a ligated polynucleotide, a recombinant polynucleotide, a polynucleotide containing a nucleic acid analogue, a methylated polynucleotide, a demethylated polynucleotide, any fragment thereof, or any combination thereof. In some embodiments, a target is a recombinant polynucleotide. In some embodiments, a target is a heterologous polynucleotide. For example, a target is a polynucleotide produced from bacterial (e.g., E. coli), yeast, mammalian, or insect cells (e.g., polynucleotides heterologous to the organisms). In some embodiments, a target is a polynucleotide with a mutation, insertion, deletion, or polymorphism.

In some embodiments, a target is an aptamer. An aptamer is an isolated nucleic acid molecule that binds with high specificity and affinity to a binding moiety or target molecule, such as a protein. An aptamer is a three dimensional structure held in certain conformation(s) that provides chemical contacts to specifically bind its given target. Although aptamers are nucleic acid based molecules, there is a fundamental difference between aptamers and other nucleic acid molecules such as genes and mRNA. In the latter, the nucleic acid structure encodes information through its linear base sequence and thus this sequence is of importance to the function of information storage. In complete contrast, aptamer function, which is based upon the specific binding of a target molecule, is not entirely dependent on a conserved linear base sequence (a non-coding sequence), but rather a particular secondary/tertiary/quaternary structure. Any coding potential that an aptamer may possess is fortuitous and is not thought to play a role in the binding of an aptamer to its cognate target. Aptamers are differentiated from naturally occurring nucleic acid sequences that bind to certain proteins. These latter sequences are naturally occurring sequences embedded within the genome of the organism that bind to a specialized sub-group of proteins that are involved in the transcription, translation, and transportation of naturally occurring nucleic acids (e.g., nucleic acid-binding proteins). Aptamers on the other hand non-naturally occurring nucleic acid molecules. While aptamers can be identified that bind nucleic acid-binding proteins, in most cases such aptamers have little or no sequence identity to the sequences recognized by the nucleic acid-binding proteins in nature. More importantly, aptamers can bind virtually any protein (not just nucleic acid-binding proteins) as well as almost any partner of interest including small molecules, carbohydrates, peptides, etc. For most partners, even proteins, a naturally occurring nucleic acid sequence to which it binds does not exist. For those partners that do have such a sequence, e.g., nucleic acid-binding proteins, such sequences will differ from aptamers as a result of the relatively low binding affinity used in nature as compared to tightly binding aptamers. Aptamers are capable of specifically binding to selected partners and modulating the partner's activity or binding interactions, e.g., through binding, aptamers may block their partner's ability to function. The functional property of specific binding to a partner is an inherent property an aptamer. An aptamer can be 6-35 kDa. An aptamer can be from 20 to 250 nucleotides. An aptamer can bind its partner with micromolar to sub-nanomolar affinity, and may discriminate against closely related targets (e.g., aptamers may selectively bind related proteins from the same gene family). In some cases, an aptamer only binds one molecule. In some cases, an aptamer binds family members of a molecule of interest. An aptamer, in some cases, binds to multiple different molecules. Aptamers are capable of using commonly seen intermolecular interactions such as hydrogen bonding, electrostatic complementarities, hydrophobic contacts, and steric exclusion to bind with a specific partner. Aptamers have a number of desirable characteristics for use as therapeutics and diagnostics including high specificity and affinity, low immunogenicity, biological efficacy, and excellent pharmacokinetic properties. An aptamer can comprise a molecular stem and loop structure formed from the hybridization of complementary polynucleotides that are covalently linked (e.g., a hairpin loop structure). The stem comprises the hybridized polynucleotides and the loop is the region that covalently links the two complementary polynucleotides. An aptamer can be a linear ribonucleic acid (e.g., linear aptamer) comprising an aptamer sequence or a circular polyribonucleic acid comprising an aptamer sequence (e.g., a circular aptamer).

In some embodiments, a target is a small molecule. For example, a small molecule can be a macrocyclic molecule, an inhibitor, a drug, or chemical compound. In some embodiments, a small molecule contains no more than five hydrogen bond donors. In some embodiments, a small molecule contains no more than ten hydrogen bond acceptors. In some embodiments, a small molecule has a molecular weight of 500 Daltons or less. In some embodiments, a small molecule has a molecular weight of from about 180 to 500 Daltons. In some embodiments, a small molecule contains an octanol-water partition coefficient lop P of no more than five. In some embodiments, a small molecule has a partition coefficient log P of from −0.4 to 5.6. In some embodiments, a small molecule has a molar refractivity of from 40 to 130. In some embodiments, a small molecule contains from about 20 to about 70 atoms. In some embodiments, a small molecule has a polar surface area of 140 Angstroms² or less.

In some embodiments, a target is a cell. For example, a target is an intact cell, a cell treated with a compound (e.g., a drug), a fixed cell, a lysed cell, or any combination thereof. In some embodiments, a target is a single cell. In some embodiments, a target is a plurality of cells.

In some embodiments, circRNA comprises a binding site to a single target or a plurality of (e.g., two or more) targets. In one embodiment, the single circRNA comprises 2, 3, 4, 5, 6, 7, 8, 9, 10, or more different binding sites for a single target. In one embodiment, the single circRNA comprises 2, 3, 4, 5, 6, 7, 8, 9, 10, or more of the same binding sites for a single target. In one embodiment, the single circRNA comprises 2, 3, 4, 5, 6, 7, 8, 9, 10, or more different binding sites for one or more different targets. In one embodiment, two or more targets are in a sample, such as a mixture or library of targets, and the sample comprises circRNA comprising two or more binding sites that bind to the two or more targets.

In some embodiments, a single target or a plurality of (e.g., two or more) targets have a plurality of binding moieties. In one embodiment, the single target may have 2, 3, 4, 5, 6, 7, 8, 9, 10, or more binding moieties. In one embodiment, two or more targets are in a sample, such as a mixture or library of targets, and the sample comprises two or more binding moieties. In some embodiments, a single target or a plurality of targets comprise a plurality of different binding moieties. For example, a plurality may include at least about 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 binding moieties.

A target can comprise a plurality of binding moieties comprising at least 2 different binding moieties. For example, a binding moiety can comprise a plurality of binding moieties comprising at least 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, or 25,000 different binding moieties.

### Binding Sites and Binding Moieties

In some instances, a circRNA comprises one binding site. A binding site can comprise an aptamer sequence. In some instances, a circRNA comprises at least two binding sites. For example, a circRNA can comprise 2, 3, 4, 5, 6, 7, 8, 9, 10, 11, 12, 13, 14, 15, 16, 17, 18, 19, 20, or more binding sites. In some embodiments, circRNA described herein is a molecular scaffold that binds one or more targets, or one or more binding moieties of one or more targets. Each target may be, but is not limited to, a different or the same nucleic acids (e.g., RNAs, DNAs, RNA-DNA hybrids), small molecules (e.g., drugs), aptamers, polypeptides, proteins, lipids, carbohydrates, antibodies, viruses, virus particles, membranes, multi-component complexes, cells, cellular moieties, any fragments thereof, and any combination thereof. In some embodiments, the one or more binding sites binds to the same target. In some embodiments, the one or more binding sites bind to one or more binding moieties of the same target. In some embodiments, the one or more binding sites bind to one or more different targets. In some embodiments, the one or more binding sites bind to one or more binding moieties of different targets. In some embodiments, a circRNA acts as a scaffold for one or more binding one or more targets. In some embodiments, a circRNA acts as a scaffold for one or more binding moieties of one or more targets. In some embodiments, a circRNA modulates cellular processes by specifically binding to one or more one or more targets. In some embodiments, a circRNA modulates cellular processes by specifically binding to one or more binding moieties of one or more targets. In some embodiments, a circRNA modulates cellular processes by specifically binding to one or more targets. In some embodiments, a circRNA described herein includes binding sites for one or more specific targets of interest. In some embodiments, circRNA includes multiple binding sites or a combination of binding sites for each target of interest. In some embodiments, circRNA includes multiple binding sites or a combination of binding sites for each binding moiety of interest. For example, a circRNA can include one or more binding sites for a polypeptide target. In some embodiments, a circRNA includes one or more binding sites for a polynucleotide target, such as a DNA or RNA, an mRNA target, an rRNA target, a tRNA target, or a genomic DNA target.

In some instances, a circRNA comprises a binding site for a single-stranded DNA. In some instances, a circRNA comprises a binding site for double-stranded DNA. In some instances, a circRNA comprises a binding site for an antibody. In some instances, a circRNA comprises a binding site for a virus particle. In some instances, a circRNA comprises a binding site for a small molecule. In some instances, a circRNA comprises a binding site that binds in or on a cell. In some instances, a circRNA comprises a binding site for a RNA-DNA hybrid. In some instances, a circRNA comprises a binding site for a methylated polynucleotide. In some instances, a circRNA comprises a binding site for an unmethylated polynucleotide. In some instances, a circRNA comprises a binding site for an aptamer. In some instances, a circRNA comprises a binding site for a polypeptide. In some instances, a circRNA comprises a binding site for a polypeptide, a protein, a protein fragment, a tagged protein, an antibody, an antibody fragment, a small molecule, a virus particle (e.g., a virus particle comprising a transmembrane protein), or a cell. In some instances, a circRNA comprises a binding site for a binding moiety on a single-stranded DNA. In some instances, a circRNA comprises a binding site for a binding moiety on a double-stranded DNA. In some instances, a circRNA comprises a binding site for a binding moiety on an antibody. In some instances, a circRNA comprises a binding site for a binding moiety on a virus particle. In some instances, a circRNA comprises a binding site for a binding moiety on a small molecule. In some instances, a circRNA comprises a binding site for a binding moiety in or on a cell. In some instances, a circRNA comprises a binding site for a binding moiety on a RNA-DNA hybrid. In some instances, a circRNA comprises a binding site for a binding moiety on a methylated polynucleotide. In some instances, a circRNA comprises a binding site for a binding moiety on an unmethylated polynucleotide. In some instances, a circRNA comprises a binding site for a binding moiety on an aptamer. In some instances, a circRNA comprises a binding site for a binding moiety on a polypeptide. In some instances, a circRNA comprises a binding site for a binding moiety on a polypeptide, a protein, a protein fragment, a tagged protein, an antibody, an antibody fragment, a small molecule, a virus particle (e.g., a virus particle comprising a transmembrane protein), or a cell.

In some instances, a binding site binds to a portion of a target comprising at least two amide bonds. In some instances, a binding site does not bind to a portion of a target comprising a phosphodiester linkage. In some instances, a portion of the target is not DNA or RNA. In some instances, a binding moiety comprises at least two amide bonds. In some instances, a binding moiety does not comprise a phosphodiester linkage. In some instances, a binding moiety is not DNA or RNA.

The circRNAs provided herein can include one or more binding sites for binding moieties on a complex. The circRNAs provided herein can include one or more binding sites for targets to form a complex. For example, the circRNAs provided herein can act as a scaffold to form a complex between a circRNA and a target. In some embodiments, a circRNA forms a complex with a single target. In some embodiments, a circRNA forms a complex with two targets. In some embodiments, a circRNA forms a complex with three targets. In some embodiments, a circRNA forms a complex with four targets. In some embodiments, a circRNA forms a complex with five or more targets. In some embodiments, a circRNA forms a complex with a complex of two or more targets. In some embodiments, a circRNA forms a complex with a complex of three or more targets. In some embodiments, two or more circRNAs form a complex with a single target. In some embodiments, two or more circRNAs form a complex with two or more targets. In some embodiments, a first circRNA forms a complex with a first binding moiety of a first target and a second different binding moiety of a second target. In some embodiments, a first circRNA forms a complex with a first binding moiety of a first target and a second circRNA forms a complex with a second binding moiety of a second target.

In some embodiments, a circRNA can include a binding site for one or more antibody-polypeptide complexes, polypeptide-polypeptide complexes, polypeptide-DNA complexes, polypeptide-RNA complexes, polypeptide-aptamer complexes, virus particle-antibody complexes, virus particle-polypeptide complexes, virus particle-DNA complexes, virus particle-RNA complexes, virus particle-aptamer complexes, cell-antibody complexes, cell-polypeptide complexes, cell-DNA complexes, cell-RNA complexes, cell-aptamer complexes, small molecule-polypeptide complexes, small molecule-DNA complexes, small molecule-aptamer complexes, small molecule-cell complexes, small molecule-virus particle complexes, and combinations thereof.

In some embodiments, a circRNA can include a binding site for one or more binding moieties on one or more antibody-polypeptide complexes, polypeptide-polypeptide complexes, polypeptide-DNA complexes, polypeptide-RNA complexes, polypeptide-aptamer complexes, virus particle-antibody complexes, virus particle-polypeptide complexes, virus particle-DNA complexes, virus particle-RNA complexes, virus particle-aptamer complexes, cell-antibody complexes, cell-polypeptide complexes, cell-DNA complexes, cell-RNA complexes, cell-aptamer complexes, small molecule-polypeptide complexes, small molecule-DNA complexes, small molecule-aptamer complexes, small molecule-cell complexes, small molecule-virus particle complexes, and combinations thereof.

In some instances, a binding site binds to a polypeptide, protein, or fragment thereof. In some embodiments, a binding site binds to a domain, a fragment, an epitope, a region, or a portion of a polypeptide, protein, or fragment thereof of a target. For example, a binding site binds to a domain, a fragment, an epitope, a region, or a portion of an isolated polypeptide, a polypeptide of a cell, a purified polypeptide, or a recombinant polypeptide. For example, a binding site binds to a domain, a fragment, an epitope, a region, or a portion of an antibody or fragment thereof. For example, a binding site binds to a domain, a fragment, an epitope, a region, or a portion of a transcription factor. For example, a binding site binds to a domain, a fragment, an epitope, a region, or a portion of a receptor. For example, a binding site binds to a domain, a fragment, an epitope, a region, or a portion of a transmembrane receptor. Binding sites may bind to a domain, a fragment, an epitope, a region, or a portion of isolated, purified, and/or recombinant polypeptides. Binding sites can bind to a domain, a fragment, an epitope, a region, or a portion of a mixture of analytes (e.g., a lysate). For example, a binding site binds to a domain, a fragment, an epitope, a region, or a portion of from a plurality of cells or from a lysate of a single cell. A binding site can bind to a binding moiety of a target. In some instances, a binding moiety is on a polypeptide, protein, or fragment thereof. In some embodiments, a binding moiety comprises a domain, a fragment, an epitope, a region, or a portion of a polypeptide, protein, or fragment thereof. For example, a binding moiety comprises a domain, a fragment, an epitope, a region, or a portion of an isolated polypeptide, a polypeptide of a cell, a purified polypeptide, or a recombinant polypeptide. For example, a binding moiety comprises a domain, a fragment, an epitope, a region, or a portion of an antibody or fragment thereof. For example, a binding moiety comprises a domain, a fragment, an epitope, a region, or a portion of a transcription factor. For example, a binding moiety comprises a domain, a fragment, an epitope, a region, or a portion of a receptor. For example, a binding moiety comprises a domain, a fragment, an epitope, a region, or a portion of a transmembrane receptor. Binding moieties may be on or comprise a domain, a fragment, an epitope, a region, or a portion of isolated, purified, and/or recombinant polypeptides. Binding moieties include binding moieties on or a domain, a fragment, an epitope, a region, or a portion of a mixture of analytes (e.g., a lysate). For example, binding moieties are on or comprise a domain, a fragment, an epitope, a region, or a portion of from a plurality of cells or from a lysate of a single cell.

In some instances, a binding site binds to a domain, a fragment, an epitope, a region, or a portion of a chemical compound (e.g., small molecule). For example, a binding binds to a domain, a fragment, an epitope, a region, or a portion of a drug. For example, a binding site binds to a domain, a fragment, an epitope, a region, or a portion of a compound. For example, a binding moiety binds to a domain, a fragment, an epitope, a region, or a portion of an organic compound. In some instances, a binding site binds to a domain, a fragment, an epitope, a region, or a portion of a small molecule with a molecular weight of 900 Daltons or less. In some instances, a binding site binds to a domain, a fragment, an epitope, a region, or a portion of a small molecule with a molecular weight of 500 Daltons or more. The portion the small molecule that the binding site binds to may be obtained, for example, from a library of naturally occurring or synthetic molecules, including a library of compounds produced through combinatorial means, i.e. a compound diversity combinatorial library. Combinatorial libraries, as well as methods for their production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference. A binding site can bind to a binding moiety of a small molecule. In some instances, a binding moiety is on or comprises a domain, a fragment, an epitope, a region, or a portion of a small molecule. For example, a binding moiety is on or comprises a domain, a fragment, an epitope, a region, or a portion of a drug. For example, a binding moiety is on or comprises a domain, a fragment, an epitope, a region, or a portion of a compound. For example, a binding moiety is on or comprises a domain, a fragment, an epitope, a region, or a portion of an organic compound. In some instances, a binding moiety is on or comprises a domain, a fragment, an epitope, a region, or a portion of a small molecule with a molecular weight of 900 Daltons or less. In some instances, a binding moiety is on or comprises a domain, a fragment, an epitope, a region, or a portion of a small molecule with a molecular weight of 500 Daltons or more. Binding moieties may be obtained, for example, from a library of naturally occurring or synthetic molecules, including a library of compounds produced through combinatorial means, i.e., a compound diversity combinatorial library. Combinatorial libraries, as well as methods for their production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

A binding site can bind to a domain, a fragment, an epitope, a region, or a portion of a member of a specific binding pair (e.g., a ligand). A binding site can bind to a domain, a fragment, an epitope, a region, or a portion of monovalent (monoepitopic) or polyvalent (polyepitopic). A binding site can bind to an antigenic or haptenic portion of a target. A binding site can bind to a domain, a fragment, an epitope, a region, or a portion of a single molecule or a plurality of molecules that share at least one common epitope or determinant site. A binding site can bind to a domain, a fragment, an epitope, a region, or a portion of a part of a cell (e.g., a bacteria cell, a plant cell, or an animal cell). A binding site can bind to a target that is in a natural environment (e.g., tissue), a cultured cell, or a microorganism (e.g., a bacterium, fungus, protozoan, or virus), or a lysed cell. A binding site can bind to a portion of a target that is modified (e.g., chemically), to provide one or more additional binding sites such as, but not limited to, a dye (e.g., a fluorescent dye), a polypeptide modifying moiety such as a phosphate group, a carbohydrate group, and the like, or a polynucleotide modifying moiety such as a methyl group. A binding site can bind to a binding moiety of a member of a specific binding pair. A binding moiety can be on or comprise a domain, a fragment, an epitope, a region, or a portion of a member of a specific binding pair (e.g., a ligand). A binding moiety can be on or comprise a domain, a fragment, an epitope, a region, or a portion of monovalent (monoepitopic) or polyvalent (polyepitopic). A binding moiety can be antigenic or haptenic. A binding moiety can be on or comprise a domain, a fragment, an epitope, a region, or a portion of a single molecule or a plurality of molecules that share at least one common epitope or determinant site. A binding moiety can be on or comprise a domain, a fragment, an epitope, a region, or a portion of a part of a cell (e.g., a bacteria cell, a plant cell, or an animal cell). A binding moiety can be either in a natural environment (e.g., tissue), a cultured cell, or a microorganism (e.g., a bacterium, fungus, protozoan, or virus), or a lysed cell. A binding moiety can be modified (e.g., chemically), to provide one or more additional binding sites such as, but not limited to, a dye (e.g., a fluorescent dye), a polypeptide modifying moiety such as a phosphate group, a carbohydrate group, and the like, or a polynucleotide modifying moiety such as a methyl group.

In some instances, a binding site binds to a domain, a fragment, an epitope, a region, or a portion of a molecule found in a sample from a host. A binding site can bind to a binding moeity of a molecule found in a sample from a host. In some instances, a binding moiety is on or comprises a domain, a fragment, an epitope, a region, or a portion of a molecule found in a sample from a host. A sample from a host includes a body fluid (e.g., urine, blood, plasma, serum, saliva, semen, stool, sputum, cerebral spinal fluid, tears, mucus, and the like). A sample can be examined directly or may be pretreated to render a binding moiety more readily detectible. Samples include a quantity of a substance from a living thing or formerly living things. A sample can be natural, recombinant, synthetic, or not naturally occurring. A binding site can bind to any of the above that is expressed from a cell naturally or recombinantly, in a cell lysate or cell culture medium, an in vitro translated sample, or an immunoprecipitation from a sample (e.g., a cell lysate). A binding moiety can be any of the above that is expressed from a cell naturally or recombinantly, in a cell lysate or cell culture medium, an in vitro translated sample, or an immunoprecipitation from a sample (e.g., a cell lysate).

In some instances, a binding site binds to a target expressed in a cell-free system or in vitro. For example, a binding site binds to a target in a cell extract. In some instances, a binding site binds to a target in a cell extract with a DNA template, and reagents for transcription and translation. A binding site can bind to a binding moiety of a target expressed in a cell-free system or in vitro. In some instances, a binding moiety of a target is expressed in a cell-free system or in vitro. For example, a binding moiety of a target is in a cell extract. In some instances, a binding moiety of a target is in a cell extract with a DNA template, and reagents for transcription and translation. Exemplary sources of cell extracts that can be used include wheat germ, Escherichia coli, rabbit reticulocyte, hyperthermophiles, hybridomas, Xenopus oocytes, insect cells, and mammalian cells (e.g., human cells). Exemplary cell-free methods that can be used to express target polypeptides (e.g., to produce target polypeptides on an array) include Protein in situ arrays (PISA), Multiple spotting technique (MIST), Self-assembled mRNA translation, Nucleic acid programmable protein array (NAPPA), nanowell NAPPA, DNA array to protein array (DAPA), membrane-free DAPA, nanowell copying and pIP-microintaglio printing, and pMAC-protein microarray copying (See Kilb et al., Eng. Life Sci. 2014, 14, 352-364).

In some instances, a binding site binds to a target that is synthesized in situ (e.g., on a solid substrate of an array) from a DNA template. A binding site can bind to binding moiety of a target that is synthesized in situ. In some instances, a binding moiety of a target is synthesized in situ (e.g., on a solid substrate of an array) from a DNA template. In some instances, a plurality of binding moieties is synthesized in situ from a plurality of corresponding DNA templates in parallel or in a single reaction. Exemplary methods for in situ target polypeptide expression include those described in Stevens, Structure 8(9): R177-R185 (2000); Katzen et al., Trends Biotechnol. 23(3):150-6. (2005); He et al., Curr. Opin. Biotechnol. 19(1):4-9. (2008); Ramachandran et al., Science 305(5680):86-90. (2004); He et al., Nucleic Acids Res. 29(15):E73-3 (2001); Angenendt et al., Mol. Cell Proteomics 5(9): 1658-66 (2006); Tao et al, Nat Biotechnol 24(10):1253-4 (2006); Angenendt et al., Anal. Chem. 76(7):1844-9 (2004); Kinpara et al., J Biochem. 136(2):149-54 (2004); Takulapalli et al., J. Proteome Res. 11(8):4382-91 (2012); He et al., Nat. Methods 5(2):175-7 (2008); Chatterjee and J. LaBaer, Curr Opin Biotech 17(4):334-336 (2006); He and Wang, Biomol Eng 24(4):375-80 (2007); and He and Taussig, J. Immunol. Methods 274(1-2):265-70 (2003).

In some instances, a binding site binds to a nucleic acid target comprising a span of at least 6 nucleotides, for example, least 8, 9, 10, 12, 15, 20, 25, 30, 40, 50, or 100 nucleotides. In some instances, a binding site binds to a protein target comprising a contiguous stretch of nucleotides. In some instances, a binding site binds to a protein target comprising a non-contiguous stretch of nucleotides. In some instances, a binding site binds to a nucleic acid target comprising a site of a mutation or functional mutation, including a deletion, addition, swap, or truncation of the nucleotides in a nucleic acid sequence. A binding site can bind to a binding moiety of a nucleic acid target. In some instances, a binding moiety of a nucleic acid target comprises a span of at least 6 nucleotides, for example, least 8, 9, 10, 12, 15, 20, 25, 30, 40, 50, or 100 nucleotides. In some instances, a binding moiety of a protein target comprises a contiguous stretch of nucleotides. In some instances, a binding moiety of a protein target comprises a non-contiguous stretch of nucleotides. In some instances, a binding moiety of a nucleic acid target comprises a site of a mutation or functional mutation, including a deletion, addition, swap, or truncation of the nucleotides in a nucleic acid sequence.

In some instances, a binding site binds to a protein target comprising a span of at least 6 amino acids, for example, least 8, 9, 10, 12, 15, 20, 25, 30, 40, 50, or 100 amino acids. In some instances, a binding site binds to a protein target comprising a contiguous stretch of amino acids. In some instances, a binding site binds to a protein target comprising a non-contiguous stretch of amino acids. In some instances, a binding site binds to a protein target comprising a site of a mutation or functional mutation, including a deletion, addition, swap, or truncation of the amino acids in a polypeptide sequence. A binding site can bind to a binding moiety of a protein target. In some instances, a binding moiety of a protein target comprises a span of at least 6 amino acids, for example, least 8, 9, 10, 12, 15, 20, 25, 30, 40, 50, or 100 amino acids. In some instances, a binding moiety of a protein target comprises a contiguous stretch of amino acids. In some instances, a binding moiety of a protein target comprises a non-contiguous stretch of amino acids. In some instances, a binding moiety of a protein target comprises a site of a mutation or functional mutation, including a deletion, addition, swap, or truncation of the amino acids in a polypeptide sequence.

In some embodiments, a binding site binds to a domain, a fragment, an epitope, a region, or a portion of a membrane bound protein. A binding site can bind to a binding moiety of a membrane bound protein. In some embodiments, a binding moiety is on or comprises a domain, a fragment, an epitope, a region, or a portion of a membrane bound protein. Exemplary membrane bound proteins include, but are not limited to, GPCRs (e.g., adrenergic receptors, angiotensin receptors, cholecystokinin receptors, muscarinic acetylcholine receptors, neurotensin receptors, galanin receptors, dopamine receptors, opioid receptors, erotonin receptors, somatostatin receptors, etc.), ion channels (e.g., nicotinic acetylcholine receptors, sodium channels, potassium channels, etc.), non-excitable and excitable channels, receptor tyrosine kinases, receptor serine/threonine kinases, receptor guanylate cyclases, growth factor and hormone receptors (e.g., epidermal growth factor (EGF) receptor), and others. The binding site can bind to a domain, a fragment, an epitope, a region, or a portion of a mutant or modified variants of membrane-bound proteins. The binding site can bind to a binding moiety of a mutant or modified variant of membrane-bound protein. The binding moiety may also be on or comprise a domain, a fragment, an epitope, a region, or a portion of a mutant or modified variants of membrane-bound proteins. For example, some single or multiple point mutations of GPCRs retain function and are involved in disease (See, e.g., Stadel et al., (1997) Trends in Pharmacological Review 18:430-37).

A binding site binds to, for example, a domain, a fragment, an epitope, a region, or a portion of a ubiquitin ligase. A binding site binds to, for example, a domain, a fragment, an epitope, a region, or a portion of a ubiquitin adaptor, proteasome adaptor, or proteasome protein. A binding site binds to, for example, a domain, a fragment, an epitope, a region, or a portion of a protein involved in endocytosis, phagocytosis, a lysosomal pathway, an autophagic pathway, macroautophagy, microautophagy, chaperone-mediated autophagy, the multivesicular body pathway, or a combination thereof. In some instance, the binding site binds to a binding moiety. A binding moiety can comprise, for example, a domain, a fragment, an epitope, a region, or a portion of a ubiquitin ligase. A binding moiety can comprise, for example, a domain, a fragment, an epitope, a region, or a portion of a ubiquitin adaptor, proteasome adaptor, or proteasome protein. A binding moiety can comprise, for example, a domain, a fragment, an epitope, a region, or a portion of a protein involved in endocytosis, phagocytosis, a lysosomal pathway, an autophagic pathway, macroautophagy, microautophagy, chaperone-mediated autophagy, the multivesicular body pathway, or a combination thereof.

A binding site binds to, for example, a domain, a fragment, an epitope, a region, or a portion of a protein associated with a disease or condition. A binding site binds to, for example, a domain, a fragment, an epitope, a region, or a portion of a proto-oncogene. A binding site binds to, for example, a domain, a fragment, an epitope, a region, or a portion of an oncogene. A binding site binds to, for example, a domain, a fragment, an epitope, a region, or a portion of a tumor suppressor gene. A binding site binds to, for example, a domain, a fragment, an epitope, a region, or a portion of an inflammatory gene (e.g., a cytokine). A binding site can bind to a binding moiety. A binding moiety can comprise, for example, a domain, a fragment, an epitope, a region, or a portion of a protein associated with a disease or condition. A binding moiety can comprise, for example, a domain, a fragment, an epitope, a region, or a portion of a proto-oncogene. A binding moiety can comprise, for example, a domain, a fragment, an epitope, a region, or a portion of an oncogene. A binding moiety can comprise, for example, a domain, a fragment, an epitope, a region, or a portion of a tumor suppressor gene. A binding moiety can comprise, for example, a domain, a fragment, an epitope, a region, or a portion of an inflammatory gene (e.g., a cytokine).

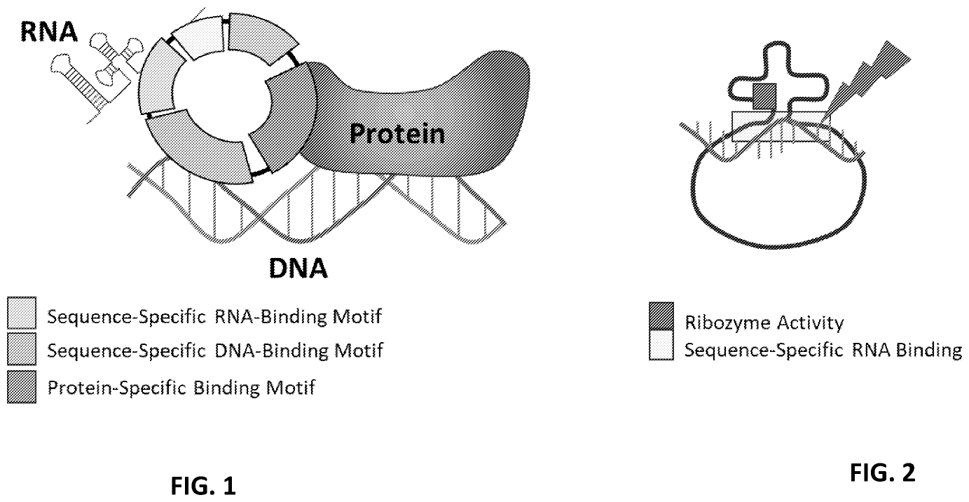

FIG. 1 shows an example of a circular polyribonucleotide with a sequence-specific RNA-binding motif, sequence-specific DNA-binding motif, and protein-specific binding motif. In some embodiments, circRNA can include other binding motifs for binding other intracellular molecules. Non-limiting examples of circRNA applications are listed in TABLE 1.

TABLE 1

| Process | MOA (example) |
|---|---|
| Directed | DNA-circRNA-Protein (pol, TF) |
| Transcription | |
| Epigenetic | DNA-circRNA-Protein (SWI/SNF) |
| Remodeling | |
| Transcriptional | circRNA-DNA |
| interference | |
| Translational | circRNA-mRNA or ribosomeI |
| interference | |
| Protein interaction | circRNA-Protein |
| inhibitor | |
| Protein Degradation | Protein-circRNA-Protein (ubiq) |
| RNA Degradation | RNA-circRNA-RNA (RNAse to RNA) |
| DNA Degradation | DNA-circRNA-Protein {DNA to |
| | DNAse) |
| Artificial Receptor | Cell Surface-circRNA-Substrate |
| Protein | Protein-circRNA-Protein/RNA |
| Translocation | |
| Cellular Fusion | Cell Surface-circRNA-Cell Surface |
| Complex | Protein-circRNA-Protein/RNA |
| Disassembly | |
| Receptor inhibition | Protein-circRNA-Substrate |
| Signal Transduction | Protein-circRNA-Protein (caspase) |
| Multi-Enzyme | Multiple Enzyems-circRNA |
| Acceleration | |
| Induction of receptor | circRNA-receptor |

RNA Binding Sites

In some embodiments, the circular polyribonucleotide comprises one or more RNA binding sites. In some embodiments, the circular polyribonucleotide includes RNA binding sites that modify expression of an endogenous gene and/or an exogenous gene. In some embodiments, the RNA binding site modulates expression of a host gene. The RNA binding site can include a sequence that hybridizes to an endogenous gene (e.g., a sequence for a miRNA, siRNA, mRNA, lncRNA, RNA, DNA, an antisense RNA, gRNA as described herein), a sequence that hybridizes to an exogenous nucleic acid such as a viral DNA or RNA, a sequence that hybridizes to an RNA, a sequence that interferes with gene transcription, a sequence that interferes with RNA translation, a sequence that stabilizes RNA or destabilizes RNA such as through targeting for degradation, or a sequence that modulates a DNA- or RNA-binding factor. In some embodiments, the circular polyribonucleotide comprises an aptamer sequence that binds to an RNA. The aptamer sequence can bind to an endogenous gene (e.g., a sequence for a miRNA, siRNA, mRNA, lncRNA, RNA, DNA, an antisense RNA, gRNA as described herein), to an exogenous nucleic acid such as a viral DNA or RNA, to an RNA, to a sequence that interferes with gene transcription, to a sequence that interferes with RNA translation, to a sequence that stabilizes RNA or destabilizes RNA such as through targeting for degradation, or to a sequence that modulates a DNA- or RNA-binding factor. The secondary structure of the aptamer sequence can bind to the RNA. The circular RNA can form a complex with the RNA by binding of the aptamer sequence to the RNA.

In some embodiments, the RNA binding site can be one of a tRNA, lncRNA, lincRNA, miRNA, rRNA, snRNA, microRNA, siRNA, piRNA, snoRNA, snRNA, exRNA, scaRNA, Y RNA, and hnRNA binding site. RNA binding sites are well-known to persons of ordinary skill in the art.

Certain RNA binding sites can inhibit gene expression through the biological process of RNA interference (RNAi). In some embodiments, the circular polyribonucleotides comprises an RNAi molecule with RNA or RNA-like structures typically having 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNA (siRNA), double-strand RNA (dsRNA), microRNA (miRNA), short hairpin RNA (shRNA), meroduplexes, and dicer substrates.

In some embodiments, the RNA binding site comprises an siRNA or an shRNA. siRNA and shRNA resemble intermediates in the processing pathway of the endogenous miRNA genes. In some embodiments, siRNA can function as miRNA and vice versa. MicroRNA, like siRNA, can use RISC to downregulate target genes, but unlike siRNA, most animal miRNA do not cleave the mRNA. Instead, miRNA reduce protein output through translational suppression or polyA removal and mRNA degradation. Known miRNA binding sites are within mRNA 3′-UTRs; miRNA seem to target sites with near-perfect complementarity to nucleotides 2-8 from the miRNA's 5′ end. This region is known as the seed region. Because siRNA and miRNA are interchangeable, exogenous siRNA can downregulate mRNA with seed complementarity to the siRNA. Multiple target sites within a 3′-UTR can give stronger downregulation.

MicroRNA (miRNA) are short noncoding RNA that bind to the 3′-UTR of nucleic acid molecules and down-regulate gene expression either by reducing nucleic acid molecule stability or by inhibiting translation. The circular polyribonucleotide can comprise one or more miRNA target sequences, miRNA sequences, or miRNA seeds. Such sequences can correspond to any miRNA.

A miRNA sequence comprises a “seed” region, i.e., a sequence in the region of positions 2-8 of the mature miRNA, which sequence has Watson-Crick complementarity to the miRNA target sequence. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA target is flanked by an adenine (A) opposed to miRNA position 1. In some embodiments, a miRNA seed can comprise 6 nucleotides (e.g., nucleotides 2-7 of the mature miRNA), wherein the seed-complementary site in the corresponding miRNA target is flanked by an adenine (A) opposed to miRNA at position 1.

The bases of the miRNA seed can be substantially complementary with the target sequence. By engineering miRNA target sequences into the circular polyribonucleotide, the circular polyribonucleotide can evade or be detected by the host's immune system, have modulated degradation, or modulated translation. This process can reduce the hazard of off target effects upon circular polyribonucleotide delivery.

The circular polyribonucleotide can include an miRNA sequence identical to about 5 to about 25 contiguous nucleotides of a target gene. In some embodiments, the miRNA sequence targets a mRNA and commences with the dinucleotide AA, comprises a GC-content of about 30%-70%, about 30%-60%, about 40%-60%, or about 45%-55%, and does not have a high percentage identity to any nucleotide sequence other than the target in the genome of the mammal in which it is to be introduced, for example, as determined by standard BLAST search.

Conversely, miRNA binding sites can be engineered out of (i.e., removed from) the circular polyribonucleotide to modulate protein expression in specific tissues. Regulation of expression in multiple tissues can be accomplished through introduction or removal or one or several miRNA binding sites.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126). MiRNA can also regulate complex biological processes, such as angiogenesis (miR-132). In the circular polyribonucleotides described herein, binding sites for miRNA that are involved in such processes can be removed or introduced, in order to tailor the expression from the circular polyribonucleotide to biologically relevant cell types or to the context of relevant biological processes. In some embodiments, the miRNA binding site includes, e.g., miR-7.

Through an understanding of the expression patterns of miRNA in different cell types, the circular polyribonucleotide described herein can be engineered for more targeted expression in specific cell types or only under specific biological conditions. Through introduction of tissue-specific miRNA binding sites, the circular polyribonucleotide can be designed for optimal protein expression in a tissue or in the context of a biological condition.

In addition, miRNA seed sites can be incorporated into the circular polyribonucleotide to modulate expression in certain cells which results in a biological improvement. An example of this is incorporation of miR-142 sites. Incorporation of miR-142 sites into the circular polyribonucleotide described herein can modulate expression in hematopoietic cells, but also reduce or abolish immune responses to a protein encoded in the circular polyribonucleotide.

In some embodiments, the circular polyribonucleotide comprises at least one miRNA, e.g., 2, 3, 4, 5, 6, or more. In some embodiments, the circular polyribonucleotide comprises an miRNA having at least about 75%, about 80%, about 85%, about 90%, about 95%, about 96%, about 97%, about 98%, about 99%, or 100% nucleotide sequence identity to any one of the nucleotide sequences or a sequence that is complementary to a target sequence.

Lists of known miRNA sequences can be found in databases maintained by research organizations, for example, Wellcome Trust Sanger Institute, Penn Center for Bioinformatics, Memorial Sloan Kettering Cancer Center, and European Molecule Biology Laboratory. RNAi molecules can be readily designed and produced by technologies known in the art. In addition, computational tools can be used to determine effective and specific sequence motifs.

In some embodiments, a circular polyribonucleotide comprises a long non-coding RNA. Long non-coding RNA (lncRNA) include non-protein coding transcripts longer than 100 nucleotides. The longer length distinguishes lncRNA from small regulatory RNA, such as miRNA, siRNA, and other short RNA. In general, the majority (˜78%) of lncRNA are characterized as tissue-specific. Divergent lncRNA that are transcribed in the opposite direction to nearby protein-coding genes (comprise a significant proportion ˜20% of total lncRNA in mammalian genomes) can regulate the transcription of the nearby gene.

The length of the RNA binding site may be between about 5 to 30 nucleotides, between about 10 to 30 nucleotides, or about 11, 12, 13, 14, 15, 16, 17, 18, 19, 20, 21, 22, 23, 24, 25, 26, 27, 28, 29, 30, or more nucleotides. The length of the RNA binding site may be between about 5 to 30 nucleotides. The length of the RNA binding site may be between about 10 to 30 nucleotides. The length of the RNA binding site may be 11 nucleotides. The length of the RNA binding site may be 12 nucleotides. The length of the RNA binding site may be 13 nucleotides. The length of the RNA binding site may be 14 nucleotides. The length of the RNA binding site may be 15 nucleotides. The length of the RNA binding site may be 16 nucleotides. The length of the RNA binding site may be 17 nucleotides. The length of the RNA binding site may be 18 nucleotides. The length of the RNA binding site may be 19 nucleotides. The length of the RNA binding site may be 20 nucleotides. The length of the RNA binding site may be 21 nucleotides. The length of the RNA binding site may be 22 nucleotides. The length of the RNA binding site may be 23 nucleotides. The length of the RNA binding site may be 24 nucleotides. The length of the RNA binding site may be 25 nucleotides. The length of the RNA binding site may be 26 nucleotides. The length of the RNA binding site may be 27 nucleotides. The length of the RNA binding site may be 28 nucleotides. The length of the RNA binding site may be 29 nucleotides. The length of the RNA binding site may be 30 nucleotides. The degree of identity of the RNA binding site to a target of interest can be at least 75%, at least 80%, at least 85%, at least 90%, or at least 95%.

In some embodiments, the circular polyribonucleotide includes one or more large intergenic non-coding RNA (lincRNA) binding sites. LincRNA make up most of the long non-coding RNA. LincRNA are non-coding transcripts and, in some embodiments, are more than about 200 nucleotides long. In some embodiments, lincRNA have an exon-intron-exon structure, similar to protein-coding genes, but do not encompass open-reading frames and do not code for proteins. LincRNA expression can be strikingly tissue-specific compared to coding genes. LincRNA are typically co-expressed with their neighboring genes to a similar extent to that of pairs of neighboring protein-coding genes. In some embodiments, the circular polyribonucleotide comprises a circularized lincRNA.

In some embodiments, the circular polyribonucleotides disclosed herein include one or more lincRNA, for example, FIRRE, LINC00969, PVT1, LINC01608, JPX, LINC01572, LINC00355, C1orf132, C3orf35, RP11-734, LINC01608, CC-499B15.5, CASC15, LINC00937, and RP11-191.

Lists of known lincRNA and lncRNA sequences can be found in databases maintained by research organizations, for example, Institute of Genomics and Integrative Biology, Diamantina Institute at the University of Queensland, Ghent University, and Sun Yat-sen University. LincRNA and lncRNA molecules can be readily designed and produced by technologies known in the art. In addition, computational tools can be used to determine effective and specific sequence motifs.

The RNA binding site can comprise a sequence that is substantially complementary, or fully complementary, to all or a fragment of an endogenous gene or gene product (e.g., mRNA). The complementary sequence can complement sequences at the boundary between introns and exons to prevent the maturation of newly-generated nuclear RNA transcripts of specific genes into mRNA for transcription. The complementary sequence may be specific to genes by hybridizing with the mRNA for that gene and prevent its translation. The RNA binding site can comprise a sequence that is antisense or substantially antisense to all or a fragment of an endogenous gene or gene product, such as DNA, RNA, or a derivative or hybrid thereof.

In some embodiments, the circular polyribonucleotide comprises a RNA binding site that has an RNA or RNA-like structure typically between about 5-5000 base pairs (depending on the specific RNA structure, e.g., miRNA 5-30 bps, lncRNA 200-500 bps) and has a nucleobase sequence identical (complementary) or nearly identical (substantially complementary) to a coding sequence in an expressed target gene within the cell.

DNA Binding Sites

In some embodiments, the circular polyribonucleotide comprises a DNA binding site, such as a sequence for a guide RNA (gRNA). In some embodiments, the circular polyribonucleotide comprises a guide RNA or a complement to a gRNA sequence. A gRNA short synthetic RNA composed of a “scaffold” sequence necessary for binding to the incomplete effector moiety and a user-defined ˜20 nucleotide targeting sequence for a genomic target. Guide RNA sequences can have a length of between 17-24 nucleotides (e.g., 19, 20, or 21 nucleotides) and complementary to the targeted nucleic acid sequence. Custom gRNA generators and algorithms can be used in the design of effective guide RNA. Gene editing can be achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNA can be effective in genome editing.

The gRNA can recognize specific DNA sequences (e.g., sequences adjacent to or within a promoter, enhancer, silencer, or repressor of a gene).

In some embodiments, the gRNA is part of a CRISPR system for gene editing. For gene editing, the circular polyribonucleotide can be designed to include one or multiple guide RNA sequences corresponding to a desired target DNA sequence. The gRNA sequences may include at least 10, 11, 12, 13, 14, 15, 16, 17, 18, 19, 20, 21, 22, 23, 24, 25, 26, 27, 28, 29, 30 or more nucleotides for interaction with Cas9 or other exonuclease to cleave DNA, e.g., Cpf1 interacts with at least about 16 nucleotides of gRNA sequence for detectable DNA cleavage.

In some embodiments, the circular polyribonucleotide comprises an aptamer sequence that can bind to DNA. The secondary structure of the aptamer sequence can bind to DNA. In some embodiments, the circular polyribonucleotide forms a complex with the DNA by binding of the aptamer sequence to the DNA.

In some embodiments, the circular polyribonucleotide includes sequences that bind a major groove of in duplex DNA. In one such instance, the specificity and stability of a triplex structure created by the circular polyribonucleotide and duplex DNA is afforded via Hoogsteen hydrogen bonds, which are different from those formed in classical Watson-Crick base pairing in duplex DNA. In one instance, the circular polyribonucleotide binds to the purine-rich strand of a target duplex through the major groove.

In some embodiments, triplex formation occurs in two motifs, distinguished by the orientation of the circular polyribonucleotide with respect to the purine-rich strand of the target duplex. In some instances, polypyrimidine sequence stretches in a circular polyribonucleotides bind to the polypurine sequence stretches of a duplex DNA via Hoogsteen hydrogen bonding in a parallel fashion (i.e., in the same 5′ to 3′, orientation as the purine-rich strand of the duplex), whereas the polypurine stretches (R) bind in an antiparallel fashion to the purine strand of the duplex via reverse-Hoogsteen hydrogen bonds. In the antiparallel, a purine motif comprises triplets of G:G-C, A:A-T, or T:A-T; whereas in the parallel, a pyrimidine motif comprises canonical triples of C+:G-C or T:A-T triplets (where C+ represents a protonated cytosine on the N3 position). Antiparallel GA and GT sequences in a circular polyribonucleotide may form stable triplexes at neutral pH, while parallel CT sequences in a circular polyribonucleotide may bind at acidic pH. N3 on cytosine in the circular polyribonucleotide may be protonated. Substitution of C with 5-methyl-C may permit binding of CT sequences in the circular polyribonucleotide at physiological pH as 5-methyl-C has a higher pK than does cytosine. For both purine and pyrimidine motifs, contiguous homopurine-homopyrimidine sequence stretches of at least 10 base pairs aid circular polyribonucleotide binding to duplex DNA, since shorter triplexes may be unstable under physiological conditions, and interruptions in sequences can destabilize the triplex structure. In some embodiments, the DNA duplex target for triplex formation includes consecutive purine bases in one strand. In some embodiments, a target for triplex formation comprises a homopurine sequence in one strand of the DNA duplex and a homopyrimidine sequence in the complementary strand.

In some embodiments, a triplex comprising a circular polyribonucleotide is a stable structure. In some embodiments, a triplex comprising a circular polyribonucleotide exhibits an increased half-life, e.g., increased by about 5%, 10%, 15%, 20%, 25%, 30%, 35%, 40%, 45%, 50%, or greater, e.g., persistence for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between.

Protein Binding Sites

In some embodiments, the circular polyribonucleotide includes one or more protein binding sites. In some embodiments, a protein binding site comprises an aptamer sequence. In one embodiment, the circular polyribonucleotide includes a protein binding site to reduce an immune response from the host as compared to the response triggered by a reference compound, e.g., a circular polyribonucleotide lacking the protein binding site, e.g., linear RNA.

In some embodiments, circular polyribonucleotides disclosed herein include one or more protein binding sites to bind a protein, e.g., a ribosome. By engineering protein binding sites, e.g., ribosome binding sites, into the circular polyribonucleotide, the circular polyribonucleotide can evade or have reduced detection by the host's immune system, have modulated degradation, or modulated translation.

In some embodiments, the circular polyribonucleotide comprises at least one immunoprotein binding site, for example, to mask the circular polyribonucleotide from components of the host's immune system, e.g., evade CTL responses. In some embodiments, the immunoprotein binding site is a nucleotide sequence that binds to an immunoprotein and aids in masking the circular polyribonucleotide as non-endogenous.

Traditional mechanisms of ribosome engagement to linear RNA involve ribosome binding to the capped 5′ end of an RNA. From the 5′ end, the ribosome migrates to an initiation codon, whereupon the first peptide bond is formed. According to the present invention, internal initiation (i.e., cap-independent) or translation of the circular polyribonucleotide does not require a free end or a capped end. Rather, a ribosome binds to a non-capped internal site, whereby the ribosome begins polypeptide elongation at an initiation codon. In some embodiments, the circular polyribonucleotide includes one or more RNA sequences comprising a ribosome binding site, e.g., an initiation codon.

In some embodiments, circular polyribonucleotides disclosed herein comprise a protein binding sequence that binds to a protein. In some embodiments, the protein binding sequence targets or localizes a circular polyribonucleotide to a specific target. In some embodiments, the protein binding sequence specifically binds an arginine-rich region of a protein.

In some embodiments, circular polyribonucleotides disclosed herein include one or more protein binding sites that each bind a target protein, e.g., acting as a scaffold to bring two or more proteins in close proximity. In some embodiments, circular polynucleotides disclosed herein comprise two protein binding sites that each bind a target protein, thereby bringing the target proteins into close proximity. In some embodiments, circular polynucleotides disclosed herein comprise three protein binding sites that each bind a target protein, thereby bringing the three target proteins into close proximity. In some embodiments, circular polynucleotides disclosed herein comprise four protein binding sites that each bind a target protein, thereby bringing the four target proteins into close proximity. In some embodiments, circular polynucleotides disclosed herein comprise five or more protein binding sites that each bind a target protein, thereby bringing five or more target proteins into close proximity. In some embodiments, the target proteins are the same. In some embodiments, the target proteins are different. In some embodiments, bringing target proteins into close proximity promotes formation of a protein complex. For example, a circular polyribonucleotide of the disclosure can act as a scaffold to promote the formation of a complex comprising one, two, three, four, five, six, seven, eight, nine, or ten target proteins, or more. In some embodiments, bringing two or more target proteins into close proximity promotes interaction of the two or more target proteins. In some embodiments, bringing two or more target proteins into close proximity modulates, promotes, or inhibits of an enzymatic reaction. In some embodiments, bringing two or more target proteins into close proximity modulates, promotes, or inhibits a signal transduction pathway.

In some embodiments, the protein binding site includes, but is not limited to, a binding site to the protein, such as ACIN1, AGO, APOBEC3F, APOBEC3G, ATXN2, AUH, BCCIP, CAPRINI, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO-, NOP58, NPM1, NUDT21, p53, PCBP2, POLR2A, PRPF8, PTBP1, RBFOX1, RBFOX2, RBFOX3, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

In some embodiments, a protein binding site is a nucleic acid sequence that binds to a protein, e.g., a sequence that can bind a transcription factor, enhancer, repressor, polymerase, nuclease, histone, or any other protein that binds DNA. In some embodiments, a protein binding site is an aptamer sequence that binds to a protein. In some embodiments, the secondary structure of the aptamer sequence binds the protein. In some embodiments, the circular RNA forms a complex with the protein by binding of the aptamer sequence to the protein.

In some embodiments, a circular RNA is conjugated to a small molecule or a part thereof, wherein the small molecule or part thereof binds to a target such as a protein. A small molecule can be conjugated to a circular RNA via a modified nucleotide, e.g., by click chemistry. Examples of small molecules that can bind to proteins include, but are not limited to 4-hydroxytamoxifen (4-OHT), AC220, Afatinib, an aminopyrazole analog, an AR antagonist, BI-7273, Bosutinib, Ceritinib, Chloroalkane, Dasatinib, Foretinib, Gefitinib, a HIF-1α-derived (R)-hydroxyproline, HJB97, a hydroxyproline-based ligand, IACS-7e, Ibrutinib, an ibrutinib derivative, JQ1, Lapatinib, an LCL161 derivative, Lenalidomide, a nutlin small molecule, OTX015, a PDE4 inhibitor, Pomalidomide, a ripk2 inhibitor, RN486, Sirt2 inhibitor 3b, SNS-032, Steel factor, a TBK1 inhibitor, Thalidomide, a thalidomide derivative, a Thiazolidinedione-based ligand, a VH032 derivative, VHL ligand 2, VHL-1, VL-269, and derivatives thereof.

In some embodiments, a circular RNA is conjugated to more than one small molecule, for instance, 2, 3, 4, 5, 6, 7, 8, 9, 10, or more small molecules. In some embodiments, a circular RNA is conjugated to 2 small molecules. In some embodiments, a circular RNA is conjugated to 3 small molecules. In some embodiments, a circular RNA is conjugated to 4 small molecules. In some embodiments, a circular RNA is conjugated to 5 small molecules. In some embodiments, a circular RNA is conjugated to 6 small molecules. In some embodiments, a circular RNA is conjugated to 7 small molecules. In some embodiments, a circular RNA is conjugated to 8 small molecules. In some embodiments, a circular RNA is conjugated to 9 small molecules. In some embodiments, a circular RNA is conjugated to 10 small molecules. In some embodiments, a circular RNA is conjugated to more than one different small molecules, for instance, 2, 3, 4, 5, 6, 7, 8, 9, 10, or more different small molecules. In some embodiments, a circular RNA is conjugated to 2 different small molecules. In some embodiments, a circular RNA is conjugated to 3 different small molecules. In some embodiments, a circular RNA is conjugated to 4 different small molecules. In some embodiments, a circular RNA is conjugated to 5 different small molecules. In some embodiments, a circular RNA is conjugated to 6 different small molecules. In some embodiments, a circular RNA is conjugated to 7 different small molecules. In some embodiments, a circular RNA is conjugated to 8 different small molecules. In some embodiments, a circular RNA is conjugated to 9 different small molecules. In some embodiments, a circular RNA is conjugated to 10 different small molecules. In some embodiments, the more than one small molecule conjugated to the circular RNA are configured to recruit their respective target proteins into proximity, which can lead to interaction between the target proteins, and/or other molecular and cellular changes. For instance, a circular RNA can be conjugated to both JQ1 and thalidomide, or derivative thereof, which can thus recruit a target protein of JQ1, e.g., BET family proteins, and a target protein of thalidomide, e.g., E3 ligase. In some cases, the circular RNA conjugated with JQ1 and thalidomide recruits a BET family protein via JQ1, or derivative thereof, tags the BET family protein with ubiquitin by E3 ligase that is recruited through thalidomide or derivative thereof, and thus leads to degradation of the tagged BET family protein.

Other Binding Sites

In some embodiments, the circular polyribonucleotide comprises one or more binding sites to a non-RNA or non-DNA target. In some embodiments, the binding site can be one of a small molecule, an aptamer, a lipid, a carbohydrate, a virus particle, a membrane, a multi-component complex, a cell, a cellular moiety, or any fragment thereof binding site. In some embodiments, the circular polyribonucleotide comprises one or more binding sites to a lipid. In some embodiments, the circular polyribonucleotide comprises one or more binding sites to a carbohydrate. In some embodiments, the circular polyribonucleotide comprises one or more binding sites to a carbohydrate. In some embodiments, the circular polyribonucleotide comprises one or more binding sites to a membrane. In some embodiments, the circular polyribonucleotide comprises one or more binding sites to a multi-component complex, e.g., ribosome, nucleosome, transcription machinery, etc.

In some embodiments, the circular polyribonucleotide comprises an aptamer sequence. The aptamer sequence can bind to any target as described herein (e.g., a nucleic acid molecule, a small molecule, a protein, a carbohydrate, a lipid, etc.). The aptamer sequence has a secondary structure that can bind the target. In some embodiments, the aptamer sequence has a tertiary structure that can bind the target. In some embodiments, the aptamer sequence has a quaternary structure that can bind the target. The circular polyribonucleotide can bind to the target via the aptamer sequence to form a complex. In some embodiments, the complex is detectable for at least 5 days. In some embodiments, the complex is detectable for at least 2 days, 3, days, 4 days, 5 days, 6 days, 7 days, 8 days, 9 days, 10 days, 11 days, 12 days, 13 days, 14 days, 15 days, 16 days.

### Sequestration

In some embodiments, circRNA described herein sequesters a target, e.g., DNA, RNA, proteins, and other cellular components to regulate cellular processes. CircRNA with binding sites for a target of interest can compete with binding of the target with an endogenous binding partner. In some embodiments, circRNA described herein sequesters miRNA. In some embodiments, circRNA described herein sequesters mRNA. In some embodiments, circRNA described herein sequesters proteins. In some embodiments, circRNA described herein sequesters ribosomes. In some embodiments, circRNA described herein sequesters other circRNA. In some embodiments, circRNA described herein sequesters non-coding RNA, lncRNA, miRNA, tRNA, rRNA, snoRNA, ncRNA, siRNA, or shRNA. In some embodiments, circRNA described herein includes a degradation element that degrades a sequestered target, e.g., DNA, RNA, protein, or other cellular component bound to the circRNA. Non-limiting examples of circRNA sequestration applications are listed in TABLE 2.

TABLE 2

| Process | MOA (example) |
|---|---|
| Transcriptional interference | circRNA-DNA |
| Translational interference | circRNA-mRNA or ribosome |
| Protein interaction inhibitor | circRNA-Protein |
| microRNA sequester | circRNA-RNA (antisense) |
| circRNA sequester | circRNA-circRNA (antisense) |
| (endogenous circRNA) | |

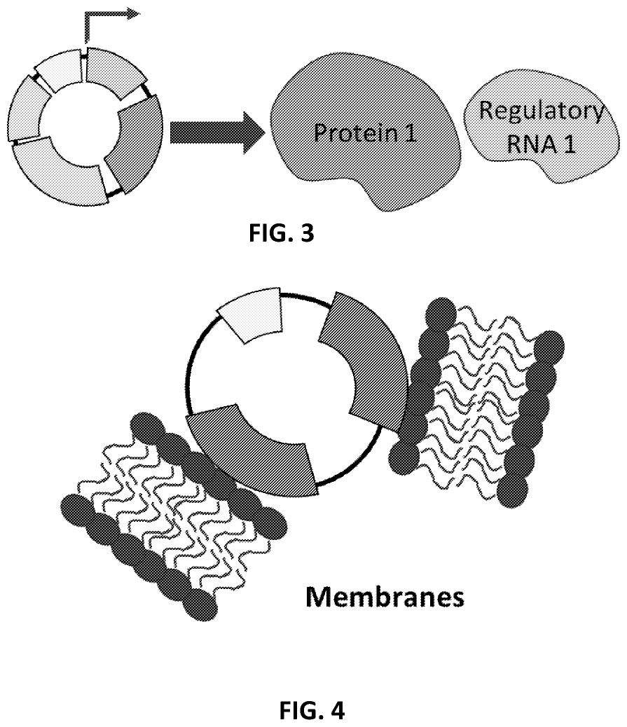

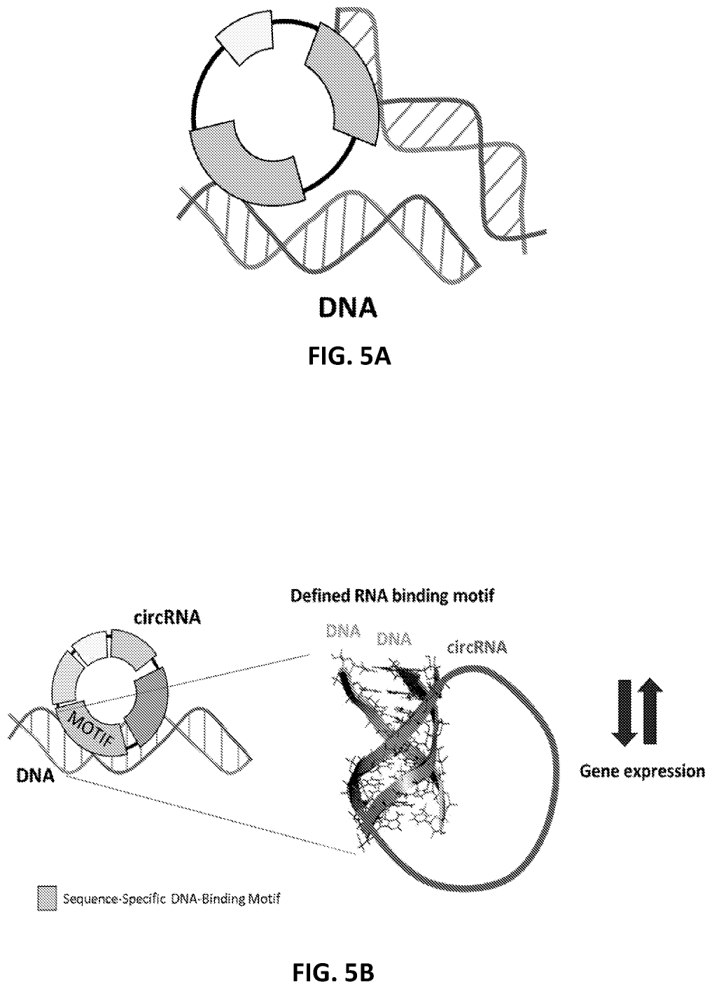

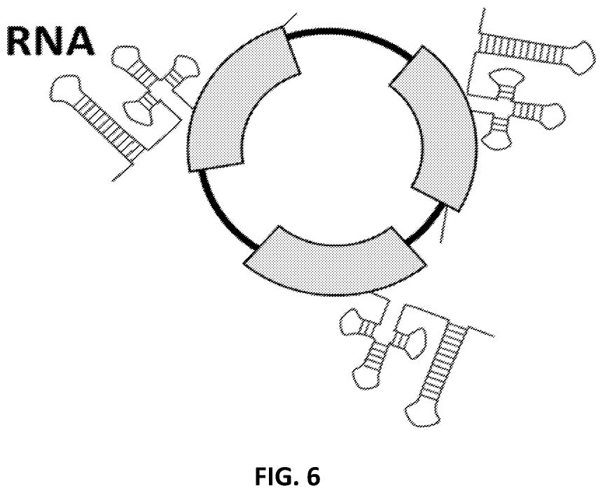

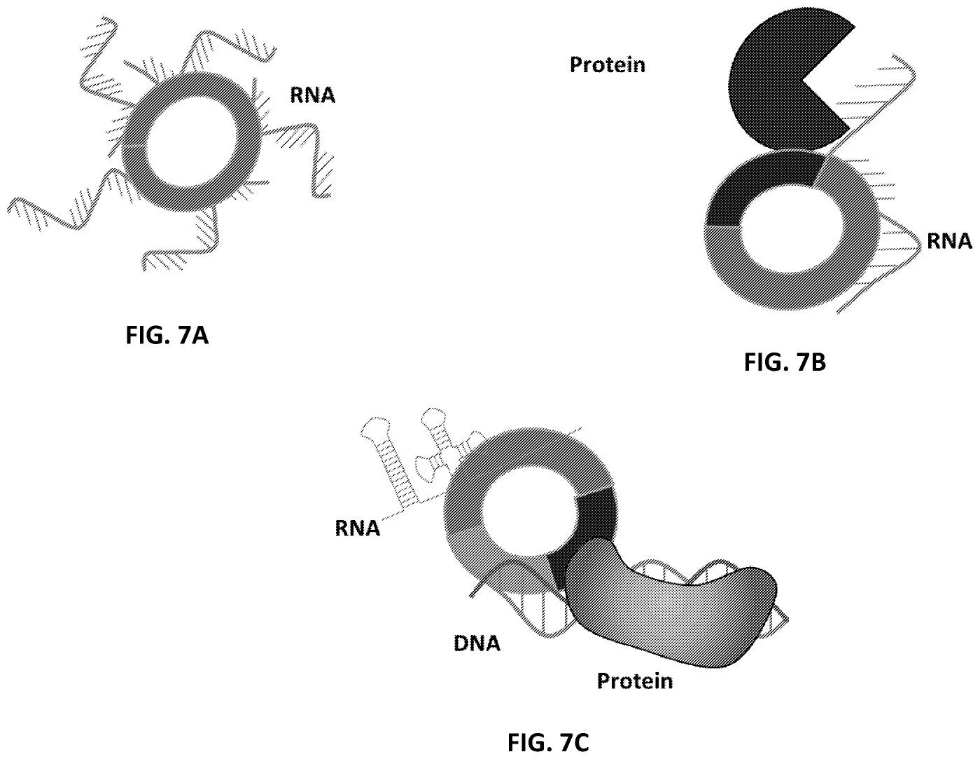

In some embodiments, any of the methods of using circRNA described herein can be in combination with a translating element. CircRNA described herein that contain a translating element can translate RNA into proteins. FIG. 3 illustrates a schematic of protein expression facilitated by a circRNA containing a sequence-specific RNA-binding motif, sequence-specific DNA-binding motif, protein-specific binding motif (Protein 1), and regulatory RNA motif (RNA 1). The regulatory RNA motif can initiate RNA transcription and protein expression.

### Untranslated Regions

In some embodiments, a circRNA as disclosed herein can comprise an encryptogen. In some embodiments, the encryptogen comprises untranslated regions (UTRs). UTRs of a gene can be transcribed but not translated. In some embodiments, a UTR can be included upstream of the translation initiation sequence of an expression sequence described herein. In some embodiments, a UTR can be included downstream of an expression sequence described herein. In some instances, one UTR for first expression sequence is the same as or continuous with or overlapping with another UTR for a second expression sequence. In some embodiments, the intron is a human intron. In some embodiments, the intron is a full length human intron, e.g., ZKSCAN1.

In some embodiments, the encryptogen enhances stability. In some embodiments, the regulatory features of a UTR can be included in the encryptogen to enhance the stability of the circular polyribonucleotide.

In some embodiments, the circular polyribonucleotide comprises a UTR with one or more stretches of adenosines and uridines embedded within. AU-rich signatures can increase turnover rates of the expression product.

Introduction, removal, or modification of UTR AU-rich elements (AREs) can be useful to modulate the stability or immunogenicity of the circular polyribonucleotide. When engineering specific circular polyribonucleotides, one or more copies of an ARE can be introduced to destabilize the circular polyribonucleotide and the copies of an ARE can decrease translation and/or decrease production of an expression product. Likewise, AREs can be identified and removed or mutated to increase the intracellular stability and thus increase translation and production of the resultant protein.

A UTR from any gene can be incorporated into the respective flanking regions of the circular polyribonucleotide. Furthermore, multiple wild-type UTRs of any known gene can be utilized. In some embodiments, artificial UTRs that are not variants of wild type genes can be used. These UTRs or portions thereof can be placed in the same orientation as in the transcript from which they were selected or can be altered in orientation or location. Hence a 5′- or 3′-UTR can be inverted, shortened, lengthened, or made chimeric with one or more other 5′- or 3′-UTRs. As used herein, the term “altered” as it relates to a UTR sequence, means that the UTR has been changed in some way in relation to a reference sequence. For example, a 3′- or 5′-UTR can be altered relative to a wild type or native UTR by the change in orientation or location as taught above or can be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double UTR, triple UTR, or quadruple UTR, such as a 5′- or 3′-UTR, can be used. As used herein, a “double” UTR is one in which two copies of the same UTR are encoded either in series or substantially in series. For example, a double beta-globin 3′-UTR can be used in some embodiments of the invention.

### Encryptogen

As described herein, a circular polyribonucleotide can comprise an encryptogen to reduce, evade, or avoid the innate immune response of a cell. In some embodiments, circular polyribonucleotides provided herein result in a reduced immune response from the host as compared to the response triggered by a reference compound, e.g., a linear polynucleotide corresponding to the described circular polyribonucleotide or a circular polyribonucleotide lacking an encryptogen. In some embodiments, the circular polyribonucleotide has less immunogenicity than a counterpart lacking an encryptogen.

In some embodiments, the circular polyribonucleotide is non-immunogenic in a mammal, e.g., a human. In some embodiments, the circular polyribonucleotide is capable of replicating in a mammalian cell, e.g., a human cell.

In some embodiments, the circular polyribonucleotide includes sequences or expression products.

In some embodiments, the circular polyribonucleotide has a half-life of at least that of a linear counterpart, e.g., linear expression sequence, or linear circular polyribonucleotide. In some embodiments, the circular polyribonucleotide has a half-life that is increased over that of a linear counterpart. In some embodiments, the half-life is increased by about 5%, 10%, 15%, 20%, 25%, 30%, 35%, 40%, 45%, 50%, or greater. In some embodiments, the circular polyribonucleotide has a half-life or persistence in a cell for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between. In certain embodiments, the circular polyribonucleotide has a half-life or persistence in a cell for no more than about 10 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time there between.

In some embodiments, the circular polyribonucleotide modulates a cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time there between.

In some embodiments, the circular polyribonucleotide is at least about 20 base pairs, at least about 30 base pairs, at least about 40 base pairs, at least about 50 base pairs, at least about 75 base pairs, at least about 100 base pairs, at least about 200 base pairs, at least about 300 base pairs, at least about 400 base pairs, at least about 500 base pairs, or at least about 1,000 base pairs. In some embodiments, the circular polyribonucleotide can be of a sufficient size to accommodate a binding site for a ribosome. One of skill in the art can appreciate that the maximum size of a circular polyribonucleotide can be as large as is within the technical constraints of producing a circular polyribonucleotide, and/or using the circular polyribonucleotide. While not being bound by theory, it is possible that multiple segments of RNA can be produced from DNA and their 5′ and 3′ free ends annealed to produce a “string” of RNA, which ultimately can be circularized when only one 5′ and one 3′ free end remains. In some embodiments, the maximum size of a circular polyribonucleotide can be limited by the ability of packaging and delivering the RNA to a target. In some embodiments, the size of a circular polyribonucleotide is a length sufficient to encode useful polypeptides, and thus, lengths of less than about 20,000 base pairs, less than about 15,000 base pairs, less than about 10,000 base pairs, less than about 7,500 base pairs, or less than about 5,000 base pairs, less than about 4,000 base pairs, less than about 3,000 base pairs, less than about 2,000 base pairs, less than about 1,000 base pairs, less than about 500 base pairs, less than about 400 base pairs, less than about 300 base pairs, less than about 200 base pairs, less than about 100 base pairs can be useful.

### Cleavage Sequences

In some embodiments, the circular polyribonucleotide includes at least one cleavage sequence. In some embodiments, the cleavage sequence is adjacent to an expression sequence. In some embodiments, the circular polyribonucleotide includes a cleavage sequence, such as in an immolating circRNA or cleavable circRNA or self-cleaving circRNA. In some embodiments, the circular polyribonucleotide comprises two or more cleavage sequences, leading to separation of the circular polyribonucleotide into multiple products, e.g., miRNAs, linear RNAs, smaller circular polyribonucleotide, etc.

In some embodiments, the cleavage sequence includes a ribozyme RNA sequence. A ribozyme (from ribonucleic acid enzyme, also called RNA enzyme or catalytic RNA) is a RNA molecule that catalyzes a chemical reaction. Many natural ribozymes catalyze either the hydrolysis of one of their own phosphodiester bonds, or the hydrolysis of bonds in other RNA, but they have also been found to catalyze the aminotransferase activity of the ribosome. Catalytic RNA can be “evolved” by in vitro methods. Similar to riboswitch activity discussed above, ribozymes and their reaction products can regulate gene expression. In some embodiments, a catalytic RNA or ribozyme can be placed within a larger non-coding RNA such that the ribozyme is present at many copies within the cell for the purposes of chemical transformation of a molecule from a bulk volume. In some embodiments, aptamers and ribozymes can both be encoded in the same non-coding RNA.

### Immolating Sequence

In some embodiments, circRNA described herein comprises immolating circRNA or cleavable circRNA or self-cleaving circRNA. CircRNA can deliver cellular components including, for example, RNA, lncRNA, lincRNA, miRNA, tRNA, rRNA, snoRNA, ncRNA, siRNA, or shRNA. In some embodiments, circRNA includes miRNA separated by (i) self-cleavable elements; (ii) cleavage recruitment sites; (iii) degradable linkers; (iv) chemical linkers; and/or (v) spacer sequences. In some embodiments, circRNA includes siRNA separated by (i) self-cleavable elements; (ii) cleavage recruitment sites (e.g., ADAR); (iii) degradable linkers (e.g., glycerol); (iv) chemical linkers; and/or (v) spacer sequences. Non-limiting examples of self-cleavable elements include hammerhead, splicing element, hairpin, hepatitis delta virus (HDV), Varkud Satellite (VS), and glmS ribozymes. Non-limiting examples of circRNA immolating applications are listed in TABLE 3.

TABLE 3

| Process | MOA (example) |
|---|---|
| miRNA delivery | microRNAs in a circular form with self |
| | cleavage element (e.g., hammerhead), cleavage |
| | recruitment (e.g., ADAR), or degradable linker |
| | (e.g., glycerol) |
| siRNA delivery | siRNAs in circular form with self |
| | cleavage element (e.g., hammerhead), cleavage |
| | recruitment (e.g., ADAR), or degradable linker |
| | (e.g., glycerol) |

### Riboswitches

In some embodiments, the circular polyribonucleotide comprises one or more riboswitches.

A riboswitch can be a part of the circular polyribonucleotide that can directly bind a small target molecule, and whose binding of the target affects RNA translation and the expression product stability and activity. Thus, the circular polyribonucleotide that includes a riboswitch can regulate the activity of the circular polyribonucleotide depending on the presence or absence of the target molecule. In some embodiments, a riboswitch has a region of aptamer-like affinity for a separate molecule. Any aptamer included within a non-coding nucleic acid can be used for sequestration of molecules from bulk volumes. In some embodiments, “(ribo)switch” activity can be used for downstream reporting of the event.

In some embodiments, the riboswitch modulates gene expression by transcriptional termination, inhibition of translation initiation, mRNA self-cleavage, and in eukaryotes, alteration of splicing pathways. The riboswitch can control gene expression through the binding or removal of a trigger molecule. Thus, subjecting a circular polyribonucleotide that includes the riboswitch to conditions that activate, deactivate, or block the riboswitch can alter gene expression. For example, gene expression can be altered as a result of termination of transcription or blocking of ribosome binding to the RNA. Binding of a trigger molecule, or an analog thereof, can reduce/prevent expression or promote/increase expression of the RNA molecule depending on the nature of the riboswitch.

In some embodiments, the riboswitch is a Cobalamin riboswitch (also B12-element), which binds adenosylcobalamin (the coenzyme form of vitamin B12) to regulate the biosynthesis and transport of cobalamin and similar metabolites.

In some embodiments, the riboswitch is a cyclic di-GMP riboswitch, which binds cyclic di-GMP to regulate a variety of genes. There are two non-structurally related classes of cyclic di-GMP riboswitch: cyclic di-GMP-I and cyclic di-GMP-II.

In some embodiments, the riboswitch is a FMN riboswitch (also RFN-element) which binds flavin mononucleotide (FMN) to regulate riboflavin biosynthesis and transport.

In some embodiments, the riboswitch is a glmS riboswitch, which cleaves itself when there is a sufficient concentration of glucosamine-6-phosphate.

In some embodiments, the riboswitch is a glutamine riboswitch, which binds glutamine to regulate genes involved in glutamine and nitrogen metabolism. Glutamine riboswitches can also bind short peptides of unknown function. Such riboswitches fall into two structurally related classes: the glnA RNA motif and Downstream-peptide motif.

In some embodiments, the riboswitch is a glycine riboswitch, which binds glycine to regulate glycine metabolism genes. It comprises two adjacent aptamer domains in the same mRNA, and is the only known natural RNA that exhibits cooperative binding.

In some embodiments, the riboswitch is a lysine riboswitch (also L-box), which binds lysine to regulate lysine biosynthesis, catabolism, and transport.

In some embodiments, the riboswitch is a preQ1 riboswitch, which binds pre-queuosine to regulate genes involved in the synthesis or transport of this precursor to queuosine. Two distinct classes of preQ1 riboswitches are preQ1-I riboswitches and preQ1-II riboswitches. The binding domain of preQ1-I riboswitches is unusually small among naturally occurring riboswitches. PreQ1-II riboswitches, which are only found in certain species in the genera Streptococcus and Lactococcus, have a completely different structure and are larger than preQ1-I riboswitches.

In some embodiments, the riboswitch is a purine riboswitch, which binds purines to regulate purine metabolism and transport. Different forms of purine riboswitches bind guanine or adenine. The specificity for either guanine or adenine depends upon Watson-Crick interactions with a single pyrimidine in the riboswitch at position Y74. In the guanine riboswitch, the single pyrimidine is cytosine (i.e., C74). In the adenine riboswitch, the single pyrimidine is uracil (i.e., U74). Homologous types of purine riboswitches can bind deoxyguanosine, but have more significant differences than a single nucleotide mutation.

In some embodiments, the riboswitch is an S-adenosylhomocysteine (SAH) riboswitch, which binds SAH to regulate genes involved in recycling SAH produced from S-adenosylmethionine (SAM) in methylation reactions.

In some embodiments, the riboswitch is an S-adenosyl methionine (SAM) riboswitch, which binds SAM to regulate methionine and SAM biosynthesis and transport. There are three distinct SAM riboswitches: SAM-I (originally called S-box), SAM-II, and the SMK box. SAM-I is widespread in bacteria. SAM-II is found only in α-, β-, and a few γ-proteobacteria. The SMK box riboswitch is found in Lactobacillales. These three varieties of riboswitch have no obvious sequence or structural similarities. A fourth variety, SAM-IV, appears to have a similar ligand-binding core to that of SAM-I, but in the context of a distinct scaffold.

In some embodiments, the riboswitch is a SAM-SAH riboswitch, which binds both SAM and SAH with similar affinities.

In some embodiments, the riboswitch is a tetrahydrofolate riboswitch, which binds tetrahydrofolate to regulate synthesis and transport genes.

In some embodiments, the riboswitch is a theophylline-binding riboswitch or a thymine pyrophosphate-binding riboswitch.

In some embodiments, the riboswitch is a glmS catalytic riboswitch from Thermoanaerobacter tengcongensis, which senses glucosamine-6 phosphate.

In some embodiments, the riboswitch is a thiamine pyrophosphate (TPP) riboswitch (also Thi-box), which binds TPP to regulate thiamine biosynthesis and transport, as well as transport of similar metabolites. The TPP riboswitch is found in eukaryotes.

In some embodiments, the riboswitch is a Moco riboswitch, which binds molybdenum cofactor, to regulate genes involved in biosynthesis and transport of this coenzyme, as well as enzymes that use molybdenum or derivatives thereof as a cofactor.

In some embodiments, the riboswitch is an adenine-sensing add-A riboswitch, found in the 5′-UTR of the adenine deaminase (add) encoding gene of Vibrio vulnificus.

### Aptazyme

In some embodiments, the circular polyribonucleotide comprises an aptazyme. Aptazyme is a switch for conditional expression in which an aptamer region is used as an allosteric control element and coupled to a region of catalytic RNA (a “ribozyme” as described below). In some embodiments, the aptazyme is active in cell type-specific translation. In some embodiments, the aptazyme is active under cell state-specific translation, e.g., virally infected cells or in the presence of viral nucleic acids or viral proteins.

A ribozyme is a RNA molecule that catalyzes a chemical reaction. Many natural ribozymes can catalyze the hydrolysis of phosphodiester bonds of the ribozyme itself or the hydrolysis of phosphodiester bonds in other RNA. Natural ribozymes can also catalyze the aminotransferase activity of the ribosome. Catalytic RNA can be “evolved” by in vitro methods. Ribozymes and reaction products of ribozymes can regulate gene expression. In some embodiments, a catalytic RNA or ribozyme can be placed within a larger, non-coding RNA such that the ribozyme is present at many copies within the cell for chemical transformation of a molecule from a bulk volume. In some embodiments, aptamers and ribozymes can both be encoded in the same non-coding RNA.

Non-limiting examples of ribozymes include hammerhead ribozyme, VL ribozyme, leadzyme, and hairpin ribozyme.

In some embodiments, the aptazyme is a ribozyme that can cleave RNA sequences and can be regulated as a result of binding a ligand or modulator. The ribozyme can be a self-cleaving ribozyme. As such, these ribozymes can combine the properties of ribozymes and aptamers.

In some embodiments, the aptazyme is included in an untranslated region of circular polyribonucleotides described herein. An aptazyme in the absence of ligand/modulator is inactive, which can allow expression of the transgene. Expression can be turned off or down-regulated by addition of the ligand. Aptazymes that are downregulated in response to the presence of a particular modulator can be used in control systems where upregulation of gene expression in response to modulator is desired.

Aptazymes can also be used to develop of systems for self-regulation of circular polyribonucleotide expression. For example, the protein product of circular polyribonucleotides described herein that is the rate determining enzyme in the synthesis of a particular small molecule can be modified to include an aptazyme that is selected to have increased catalytic activity in the presence of the small molecule to provide an autoregulatory feedback loop for synthesis of the molecule. Alternatively, the aptazyme activity can be selected sense accumulation of the protein product from the circular polyribonucleotide, or any other cellular macromolecule.

In some embodiments, the circular polyribonucleotide can include an aptamer sequence. Non-limiting examples of aptamers include RNA aptamers that bind lysozyme, Toggle-25t (an RNA aptamer containing 2′-fluoropyrimidine nucleotides that binds thrombin with high specificity and affinity), RNA-Tat that binds human immunodeficiency virus trans-acting responsive element (HIV TAR), RNA aptamers that bind hemin, RNA aptamers that bind interferon γ, RNA aptamer binding vascular endothelial growth factor (VEGF), RNA aptamers that bind prostate specific antigen (PSA), RNA aptamers that bind dopamine, and RNA aptamers that bind heat shock factor 1 (HSF1).

In some embodiments, circRNA described herein can be used for transcription and replication of RNA. For example, circRNA can be used to encode non-coding RNA, lncRNA, miRNA, tRNA, rRNA, snoRNA, ncRNA, siRNA, or shRNA. In some embodiments, circRNA can include anti-sense miRNA and a transcriptional element. After transcription, such circRNA can produce functional, linear miRNAs. Non-limiting examples of circRNA expression and modulation applications are listed in TABLE 4.

TABLE 4

| Process | MOA (example) |
|---|---|
| Combinational therapy of | Inhibition of one protein and |
| inhibition & translation | supplementation of another (or same) |

### Replication Element

The circular polyribonucleotide can encode a sequence and/or motif useful for replication. Replication of a circular polyribonucleotide can occur by generating a complement circular polyribonucleotide. In some embodiments, the circular polyribonucleotide includes a motif to initiate transcription, where transcription is driven by either endogenous cellular machinery (DNA-dependent RNA polymerase) or an RNA-depended RNA polymerase encoded by the circular polyribonucleotide. The product of rolling-circle transcriptional event can be cut by a ribozyme to generate either complementary or propagated circular polyribonucleotide at unit length. The ribozymes can be encoded by the circular polyribonucleotide, its complement, or by an RNA sequence in trans. In some embodiments, the encoded ribozymes can include a sequence or motif that regulates (inhibits or promotes) activity of the ribozyme to control circRNA propagation. In some embodiments, unit-length sequences can be ligated into a circular form by a cellular RNA ligase. In some embodiments, the circular polyribonucleotide includes a replication element that aids in self-amplification. Examples of such replication elements include HDV replication domains and replication competent circular RNA sense and/or antisense ribozymes, such as antigenomic 5′-CGGGUCGGCAUGGCAUCUCCACCUCCUCGCGGUCCGACCUGGGCAUCCGAAGGAGGACGC ACGUCCACUCGGAUGGCUAAGGGAGAGCCA-3′ or genomic 5′-UGGCCGGCAUGGUCCCAGCCUCCUCGCUGGCGCCGGCUGGGCAACAUUCCGAGGGGACCG UCCCCUCGGUAAUGGCGAAUGGGACCCA-3′.

In some embodiments, the circular polyribonucleotide includes at least one cleavage sequence as described herein to aid in replication. A cleavage sequence within the circular polyribonucleotide can cleave long transcripts replicated from the circular polyribonucleotide to a specific length that can subsequently circularize to form a complement to the circular polyribonucleotide.

In another embodiment, the circular polyribonucleotide includes at least one ribozyme sequence to cleave long transcripts replicated from the circular polyribonucleotide to a specific length, where another encoded ribozyme cuts the transcripts at the ribozyme sequence. Circularization forms a complement to the circular polyribonucleotide.

In some embodiments, the circular polyribonucleotide is substantially resistant to degradation, e.g., by exonucleases.

In some embodiments, the circular polyribonucleotide replicates within a cell. In some embodiments, the circular polyribonucleotide replicates within in a cell at a rate of between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage there between. In some embodiments, the circular polyribonucleotide is replicates within a cell and is passed to daughter cells. In some embodiments, a cell passes at least one circular polyribonucleotide to daughter cells with an efficiency of at least 25%, 50%, 60%, 70%, 80%, 85%, 90%, 95%, or 99%. In some embodiments, cell undergoing meiosis passes the circular polyribonucleotide to daughter cells with an efficiency of at least 25%, 50%, 60%, 70%, 80%, 85%, 90%, 95%, or 99%. In some embodiments, a cell undergoing mitosis passes the circular polyribonucleotide to daughter cells with an efficiency of at least 25%, 50%, 60%, 70%, 80%, 85%, 90%, 95%, or 99%.

In some embodiments, the circular polyribonucleotide replicates within the host cell. In some embodiments, the circular polyribonucleotide is capable of replicating in a mammalian cell, e.g., human cell.

While in some embodiments the circular polyribonucleotide replicates in the host cell, the circular polyribonucleotide does not integrate into the genome of the host, e.g., with the host's chromosomes. In some embodiments, the circular polyribonucleotide has a negligible recombination frequency, e.g., with the host's chromosomes. In some embodiments, the circular polyribonucleotide has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host's chromosomes.

### Expression Sequences

Peptides or Polypeptides

In some embodiments, the circular polyribonucleotide comprises a sequence that encodes a peptide or polypeptide.

The polypeptide can be linear or branched. The polypeptide can have a length from about 5 to about 4000 amino acids, about 15 to about 3500 amino acids, about 20 to about 3000 amino acids, about 25 to about 2500 amino acids, about 50 to about 2000 amino acids, or any range there between. In some embodiments, the polypeptide has a length of less than about 4000 amino acids, less than about 3500 amino acids, less than about 3000 amino acids, less than about 2500 amino acids, or less than about 2000 amino acids, less than about 1500 amino acids, less than about 1000 amino acids, less than about 900 amino acids, less than about 800 amino acids, less than about 700 amino acids, less than about 600 amino acids, less than about 500 amino acids, less than about 400 amino acids, less than about 300 amino acids, or less can be useful.

In some embodiments, the circular polyribonucleotide comprises one or more RNA sequences, each of which can encode a polypeptide. The polypeptide can be produced in substantial amounts. As such, the polypeptide can be any proteinaceous molecule that can be produced. A polypeptide can be a polypeptide that can be secreted from a cell, or localized to the cytoplasm, nucleus or membrane compartment of a cell.

In some embodiments, the circular polyribonucleotide includes a sequence encoding a protein e.g., a therapeutic protein. Some examples of therapeutic proteins can include, but are not limited to, an protein replacement, protein supplementation, vaccination, antigens (e.g., tumor antigens, viral, and bacterial), hormones, cytokines, antibodies, immunotherapy (e.g., cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component thereof.

Regulatory Sequences

In some embodiments, the regulatory sequence is a promoter. In some embodiments, the circular polyribonucleotide includes at least one promoter adjacent to at least one expression sequence. In some embodiments, the circular polyribonucleotide includes a promoter adjacent each expression sequence. In some embodiments, the promoter is present on one or both sides of each expression sequence, leading to separation of the expression products, e.g., peptide(s) and or polypeptide(s).

The circular polyribonucleotide can modulate expression of RNA encoded by a gene. Because multiple genes can share some degree of sequence homology with each other, the circular polyribonucleotide can be designed to target a class of genes with sufficient sequence homology. In some embodiments, the circular polyribonucleotide can contain a sequence that has complementarity to sequences that are shared amongst different gene targets or are unique for a specific gene target. In some embodiments, the circular polyribonucleotide can be designed to target conserved regions of an RNA sequence having homology between several genes thereby targeting several genes in a gene family. In some embodiments, the circular polyribonucleotide can be designed to target a sequence that is unique to a specific RNA sequence of a single gene.

In some embodiments, the expression sequence has a length less than 5000 bps (e.g., less than about 5000 bps, 4000 bps, 3000 bps, 2000 bps, 1000 bps, 900 bps, 800 bps, 700 bps, 600 bps, 500 bps, 400 bps, 300 bps, 200 bps, 100 bps, 50 bps, 40 bps, 30 bps, 20 bps, 10 bps, or less). In some embodiments, the expression sequence has, independently or in addition to, a length greater than 10 bps (e.g., at least about 10 bps, 20 bps, 30 bps, 40 bps, 50 bps, 60 bps, 70 bps, 80 bps, 90 bps, 100 bps, 200 bps, 300 bps, 400 bps, 500 bps, 600 bps, 700 bps, 800 bps, 900 bps, 1000 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb or greater).

In some embodiments, the expression sequence comprises one or more of the features described herein, e.g., a sequence encoding one or more peptides or proteins, one or more regulatory nucleic acids, one or more non-coding RNA, and other expression sequences.

Internal Ribosome Entry Site (IRES)

In some embodiments, the circular polyribonucleotides described herein comprise an internal ribosome entry site (IRES) element. A suitable IRES element can contain an RNA sequence capable of engaging a eukaryotic ribosome. In some embodiments, the IRES element is at least about 50 base pairs, at least about 100 base pairs, at least about 200 base pairs, at least about 250 base pairs, at least about 350 base pairs, or at least about 500 base pairs. In some embodiments, the IRES element is derived from the DNA of an organism including, but not limited to, a virus, a mammal, and a Drosophila. Viral DNA can be derived from, for example, picornavirus cDNA, encephalomyocarditis virus (EMCV) cDNA, and poliovirus cDNA. In some embodiments, Drosophila DNA from which an IRES element is derived can include, for example, an Antennapedia gene from Drosophila melanogaster.

In some embodiments, circular polyribonucleotides described herein include at least one IRES flanking at least one (e.g., 2, 3, 4, 5 or more) expression sequence. In some embodiments, the IRES can flank both sides of at least one (e.g., 2, 3, 4, 5 or more) expression sequence. In some embodiments, circular polyribonucleotides can include one or more IRES sequences on one or both sides of each expression sequence, leading to separation of the resulting peptide(s) and or polypeptide(s).

Translation Initiation Sequence

In some embodiments, the circular polyribonucleotide encodes a polypeptide and can comprise a translation initiation sequence, e.g., a start codon. In some embodiments, the translation initiation sequence includes a Kozak or Shine-Dalgarno sequence. In some embodiments, the circular polyribonucleotide includes the translation initiation sequence, e.g., Kozak sequence, adjacent to an expression sequence. In some embodiments, the translation initiation sequence, e.g., Kozak sequence, is present on one or both sides of each expression sequence, leading to separation of the expression products. In some embodiments, the circular polyribonucleotide includes at least one translation initiation sequence adjacent to an expression sequence.

Natural 5′-UTRs can bear features that play a role in translation initiation. Natural 5′-UTRs can harbor signatures like Kozak sequences, which can be involved in the process by which the ribosome initiates translation of many genes. Kozak sequences have the consensus CCR(A/G)CCAUGG, where R is a purine (adenine or guanine) three bases upstream of the start codon (AUG), which is followed by another “G”. 5′-UTR can also form secondary structures that are involved in elongation factor binding.

The circular polyribonucleotide can include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60, or more than 60 start codons. Translation can initiate on the first start codon or initiate downstream of the first start codon.

In some embodiments, the circular polyribonucleotide can initiate at a codon that is not the first start codon, e.g., AUG. Translation of the circular polyribonucleotide can initiate at an alternative translation initiation sequence, such as, but not limited to, ACG, AGG, AAG, CTG/CUG, GTG/GUG, ATA/AUA, ATT/AUU, TTG/UUG. In some embodiments, translation begins at an alternative translation initiation sequence under selective conditions, e.g., stress induced conditions. As a non-limiting example, the translation of the circular polyribonucleotide can begin at alternative translation initiation sequence, such as ACG. As another non-limiting example, the circular polyribonucleotide translation can begin at alternative translation initiation sequence, CTG/CUG. As yet another non-limiting example, the circular polyribonucleotide translation can begin at alternative translation initiation sequence, GTG/GUG. As yet another non-limiting example, the circular polyribonucleotide can begin translation at a repeat-associated non-AUG (RAN) sequence, such as an alternative translation initiation sequence that includes short stretches of repetitive RNA, e.g., CGG, GGGGCC, CAG, CTG.

Nucleotides flanking a codon that initiates translation can affect the translation efficiency, the length and/or the structure of the circular polyribonucleotide. Masking any of the nucleotides flanking a codon that initiates translation can be used to alter the position of translation initiation, translation efficiency, length, and/or structure of the circular polyribonucleotide.

In some embodiments, a masking agent can be used near the start codon or alternative start codon in order to mask or hide the codon to reduce the probability of translation initiation at the masked start codon or alternative start codon. Non-limiting examples of masking agents include antisense locked nucleic acids (LNA) oligonucleotides and exon-junction complexes (EJCs). In some embodiments, a masking agent can be used to mask a start codon of the circular polyribonucleotide in order to increase the likelihood that translation will initiate at an alternative start codon.

In some embodiments, translation is initiated under selective conditions, such as, but not limited to, viral induced selection in the presence of GRSF-1 and the circular polyribonucleotide includes GRSF-1 binding sites.

In some embodiments, translation is initiated by eukaryotic initiation factor 4A (eIF4A) treatment with Rocaglates. Translation can be repressed by blocking 43S scanning, leading to premature, upstream translation initiation and reduced protein expression from transcripts bearing the RocA-eIF4A target sequence.

Termination Sequence

In some embodiments, the circular polyribonucleotide includes one or more expression sequences and each expression sequence can have a termination sequence. In some embodiments, the circular polyribonucleotide includes one or more expression sequences and the expression sequences lack a termination sequence, such that the circular polyribonucleotide is continuously translated. Exclusion of a termination sequence can result in rolling circle translation or continuous production of expression product, e.g., peptides or polypeptides, due to lack of ribosome stalling or fall-off. In such an embodiment, rolling circle translation produces a continuous expression product through each expression sequence.

In some embodiments, the circular polyribonucleotide includes a stagger sequence. To avoid production of a continuous expression product, e.g., peptide or polypeptide, while maintaining rolling circle translation, a stagger sequence can be included to induce ribosomal pausing during translation. The stagger sequence can include a 2A-like or CHYSEL (cis-acting hydrolase element) sequence. In some embodiments, the stagger element encodes a sequence with a C-terminal consensus sequence that is X1X2X3EX5NPGP, where X1 is absent or G or H, X2 is absent or D or G, X3 is D or V or I or S or M, and X5 is any amino acid. In some embodiments, this sequence comprises a non-conserved sequence of amino-acids with a strong alpha-helical propensity followed by the consensus sequence -D(V/I)ExNPG P, where x=any amino acid. Some nonlimiting examples of stagger elements includes GDVESNPGP, GDIEENPGP, VEPNPGP, IETNPGP, GDIESNPGP, GDVELNPGP, GDIETNPGP, GDVENPGP, GDVEENPGP, GDVEQNPGP, IESNPGP, GDIELNPGP, HDIETNPGP, HDVETNPGP, HDVEMNPGP, GDMESNPGP, GDVETNPGP, GDIEQNPGP, and DSEFNPGP.

In some embodiments, the circular polyribonucleotide includes a termination sequence at the end of one or more expression sequences. In some embodiments, one or more expression sequences lacks a termination sequence. Generally, termination sequences include an in-frame nucleotide triplet that signals termination of translation, e.g., UAA, UGA, UAG. In some embodiments, one or more termination sequences in the circular polyribonucleotide are frame-shifted termination sequences, such as but not limited to, off-frame or −1 and +1 shifted reading frames (e.g., hidden stop) that can terminate translation. Frame-shifted termination sequences include nucleotide triples, TAA, TAG, and TGA that appear in the second and third reading frames of an expression sequence. Frame-shifted termination sequences can be important in preventing misreads of mRNA, which is often detrimental to the cell.

In some embodiments, a stagger sequence described herein can terminate translation and/or cleave an expression product between G and P of the consensus sequence described herein. As one non-limiting example, the circular polyribonucleotide includes at least one stagger sequence to terminate translation and/or cleave the expression product. In some embodiments, the circular polyribonucleotide includes a stagger sequence adjacent to at least one expression sequence. In some embodiments, the circular polyribonucleotide includes a stagger sequence after each expression sequence. In some embodiments, the circular polyribonucleotide includes a stagger sequence is present on one or both sides of each expression sequence, leading to translation of individual peptide(s) and or polypeptide(s) from each expression sequence.

PolyA Sequence

In some embodiments, the circular polyribonucleotide includes a poly-A sequence. In some embodiments, the length of a poly-A sequence is greater than 10 nucleotides in length. In some embodiments, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is from about 10 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A sequence is designed relative to the length of the overall circular polyribonucleotide. The design can be based on the length of the coding region, the length of a particular feature or region (such as the first or flanking regions), or based on the length of the ultimate product expressed from the circular polyribonucleotide. In this context, the poly-A sequence can be 10, 20, 30, 40, 50, 60, 70, 80, 90, or 100% greater in length than the circular polyribonucleotide or a feature thereof. The poly-A sequence can also be designed as a fraction of the circular polyribonucleotide. In this context, the poly-A sequence can be 10%, 20%, 30%, 40%, 50%, 60%, 70%, 80%, 90%, or more of the total length of the construct or the total length of the construct minus the poly-A sequence. Further, engineered binding sites and conjugation of circular polyribonucleotide for Poly-A binding protein can enhance expression.

In some embodiments, the circular polyribonucleotide is designed to include a polyA-G quartet. The G-quartet is a cyclic hydrogen bonded array of four guanine nucleotides that can be formed by G-rich sequences in both DNA and RNA. In some embodiments, the G-quartet can be incorporated at the end of the poly-A sequence. The resultant circular polyribonucleotide construct can be assayed for stability, protein production, and/or other parameters including half-life at various time points. In some embodiments, the polyA-G quartet can result in protein production equivalent to at least 75% of that seen using a poly-A sequence of 120 nucleotides alone.

### Other Sequences

In some embodiments, the circular polyribonucleotide further includes another nucleic acid sequence. In some embodiments, the circular polyribonucleotide can include DNA, RNA, or artificial nucleic acid sequences. The other sequences can include, but are not limited to, genomic DNA, cDNA, or sequences that encode tRNA, mRNA, rRNA, miRNA, gRNA, siRNA, or other RNAi molecules. In some embodiments, the circular polyribonucleotide includes a sequence encoding an siRNA to target a different locus or loci of the same gene expression product as the circular polyribonucleotide. In some embodiments, the circular polyribonucleotide includes a sequence encoding an siRNA to target a different gene expression product as the circular polyribonucleotide.

In some embodiments, the circular polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular polyribonucleotide lacks a termination sequence. In some embodiments, the circular polyribonucleotide lacks an internal ribosomal entry site. In some embodiments, the circular polyribonucleotide lacks degradation susceptibility by exonucleases. In some embodiments, the circular polyribonucleotide lacks binding to cap-binding proteins. In some embodiments, the circular polyribonucleotide lacks a 5′ cap.

In some embodiments, the circular polyribonucleotide comprises one or more of the following sequences: a sequence that encodes one or more miRNA, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNA, shRNA), and a sequence that encodes a therapeutic mRNA or protein.

The other sequence can have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range there between.

As a result of its circularization, the circular polyribonucleotide can include certain characteristics that distinguish it from linear RNA. For example, the circular polyribonucleotide is less susceptible to degradation by exonuclease as compared to linear RNA. As such, the circular polyribonucleotide is more stable than a linear RNA, especially when incubated in the presence of an exonuclease. The increased stability of the circular polyribonucleotide compared with linear RNA makes circular polyribonucleotide more useful as a cell transforming reagent to produce polypeptides and can be stored more easily and for longer than linear RNA. The stability of the circular polyribonucleotide treated with exonuclease can be tested using methods standard in art which determine whether RNA degradation has occurred (e.g., by gel electrophoresis).

Moreover, unlike linear RNA, the circular polyribonucleotide is less susceptible to dephosphorylation when the circular polyribonucleotide is incubated with phosphatase, such as calf intestine phosphatase.

Nucleotide Spacer Sequences

In some embodiments, the circular polyribonucleotide comprises a spacer sequence.

The spacer can be a nucleic acid molecule having low GC content, for example less than 65%, 60%, 55%, 50%, 55%, 50%, 45%, 40%, 39%, 38%, 37%, 36%, 35%, 34%, 33%, 32%, 31%, 30%, 29%, 28%, 27%, 26%, 25%, 24%, 23%, 22%, 20%, 19%, 18%, 17%, 16%, 15%, 14%, 13%, 12%, 11%, 10%, 9%, 8%, 7%, 6%, 5%, 4%, 3%, 2% or 1%, across the full length of the spacer, or across at least 50%, 60%, 70%, 80%, 90%, 91%, 92%, 93%, 94%, 95%, 96%, 97%, 98% or 99% contiguous nucleic acid residues of the spacer. In some embodiments, the spacer is substantially free of a secondary structure, such as less than 40 kcal/mol, less than −39, −38, −37, −36, −35, −34, −33, −32, −31, −30, −29, −28, −27, −26, −25, −24, −23, −22, −20, −19, −18, −17, −16, −15, −14, −13, −12, −11, −10, −9, −8, −7, −6, −5, −4, −3, −2 or −1 kcal/mol. The spacer can include a nucleic acid, such as DNA or RNA.

The spacer sequence can encode an RNA sequence, and preferably a protein or peptide sequence, including a secretion signal peptide.

The spacer sequence can be non-coding. Where the spacer is a non-coding sequence, a start codon can be provided in the coding sequence of an adjacent sequence. In some embodiments, it is envisaged that the first nucleic acid residue of the coding sequence can be the A residue of a start codon, such as AUG. Where the spacer encodes an RNA or protein or peptide sequence, a start codon can be provided in the spacer sequence.

In some embodiments, the spacer is operably linked to another sequence described herein.

Non-Nucleic Acid Linkers

The circular polyribonucleotide described herein can also comprise a non-nucleic acid linker. In some embodiments, the circular polyribonucleotide described herein has a non-nucleic acid linker between one or more of the sequences or elements described herein. In some embodiments, one or more sequences or elements described herein are linked with the linker. The non-nucleic acid linker can be a chemical bond, e.g., one or more covalent bonds or non-covalent bonds. In some embodiments, the non-nucleic acid linker is a peptide or protein linker. Such a linker can be between 2-30 amino acids, or longer. The linker includes flexible, rigid or cleavable linkers described herein.

The most commonly used flexible linkers have sequences consisting primarily of stretches of Gly and Ser residues (“GS” linker). Flexible linkers can be useful for joining domains that require a certain degree of movement or interaction and can include small, non-polar (e.g., Gly) or polar (e.g., Ser or Thr) amino acids. Incorporation of Ser or Thr can also maintain the stability of the linker in aqueous solutions by forming hydrogen bonds with the water molecules, and therefore reduce unfavorable interactions between the linker and the protein moieties.

Rigid linkers are useful to keep a fixed distance between domains and to maintain their independent functions. Rigid linkers can also be useful when a spatial separation of the domains is critical to preserve the stability or bioactivity of one or more components in the fusion. Rigid linkers can have an alpha helix-structure or Pro-rich sequence, (XP)ₙ, with X designating any amino acid, preferably Ala, Lys, or Glu.

Cleavable linkers can release free functional domains in vivo. In some embodiments, linkers can be cleaved under specific conditions, such as the presence of reducing reagents or proteases. In vivo cleavable linkers can utilize the reversible nature of a disulfide bond. One example includes a thrombin-sensitive sequence (e.g., PRS) between the two Cys residues. In vitro thrombin treatment of CPRSC results in the cleavage of the thrombin-sensitive sequence, while the reversible disulfide linkage remains intact. In vivo cleavage of linkers in fusions can also be carried out by proteases that are expressed in vivo under pathological conditions (e.g., cancer or inflammation), in specific cells or tissues, or constrained within certain cellular compartments. The specificity of many proteases offers slower cleavage of the linker in constrained compartments.

Examples of linking molecules include a hydrophobic linker, such as a negatively charged sulfonate group; lipids, such as a poly (—CH₂-ipids, such as a poly (—CHe g polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more polypeptides. Non-covalent linkers are also included, such as hydrophobic lipid globules to which the polypeptide is linked, for example through a hydrophobic region of the polypeptide or a hydrophobic extension of the polypeptide, such as a series of residues rich in leucine, isoleucine, valine, or perhaps also alanine, phenylalanine, or even tyrosine, methionine, glycine or other hydrophobic residue. The polypeptide can be linked using charge-based chemistry, such that a positively charged moiety of the polypeptide is linked to a negative charge of another polypeptide or nucleic acid.

### Modifications

In some aspects, the invention described herein comprises compositions and methods of using and making modified circular polyribonucleotides, and delivery of modified circular polyribonucleotides. The term “modified nucleotide” can refer to any nucleotide analog or derivative that has one or more chemical modifications to the chemical composition of an unmodified natural ribonucleotide, such as a natural unmodified nucleotide adenosine (A), uridine (U), guanine (G), cytidine (C) as shown by the chemical formulae in TABLE 5, and monophosphate. The chemical modifications of the modified ribonucleotide can be modifications to any one or more functional groups of the ribonucleotide, such as, the sugar the nucleobase, or the internucleoside linkage (e.g. to a linking phosphate/to a phosphodiester linkage/to the phosphodiester backbone).

TABLE 5
Unmodified Natural Ribonucleosides

| Ribonucleoside | IUPAC name | Chemical Formula |
|---|---|---|
| Adenosine | (2R,3R,4S,5R)-2- (6-amino- 9H-purin-9-yl)-5- (hydroxymethyl) oxolane-3,4- diol | 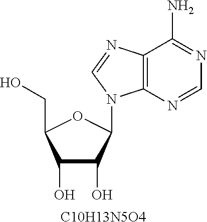 |
| Uridine | 1-[(3R,4S,5R)-3,4- dihydroxy-5- (hydroxymethyl) oxolan-2- yl]pyrimidine- 2,4-dione | 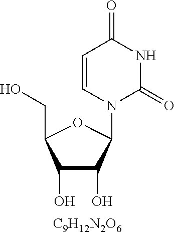 |
| Guanine | 2-amino-9H-purin- 6(1H)-one | 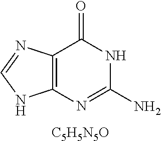 |
| Cytidine | 4-amino-1- [(2R,3R,4S,5R)- 3,4-dihydroxy-5- (hydroxymethyl) oxolan-2- yl]pyrimidin-2 (1H)-one | 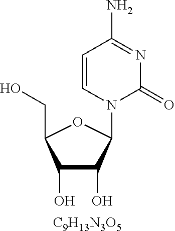 |

The circular polyribonucleotide can include one or more substitutions, insertions and/or additions, deletions, and covalent modifications with respect to reference sequences, in particular, the parent polyribonucleotide, are included within the scope of this invention. In some embodiments, the circular polyribonucleotide includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The circular polyribonucleotide can include any useful modification, such as to the sugar, the nucleobase, or the internucleoside linkage (e.g., to a linking phosphate/to a phosphodiester linkage/to the phosphodiester backbone). One or more atoms of a pyrimidine nucleobase can be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications can be modifications of ribonucleic acids (RNA) to deoxyribonucleic acids (DNA), threose nucleic acids (TNA), glycol nucleic acids (GNA), peptide nucleic acids (PNA), locked nucleic acids (LNA) or hybrids thereof). Additional modifications are described herein.

In some embodiments, the circular polyribonucleotide includes at least one N(6)methyladenosine (m6A) modification to increase translation efficiency.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

“Pseudouridine” refers, in another embodiment, to m¹acp³Ψ (1-methyl-3-(3-amino-3-carboxypropyl) pseudouridine. In another embodiment, the term refers to m¹Ψ (1-methylpseudouridine). In another embodiment, the term refers to Ψm (2′-O-methylpseudouridine. In another embodiment, the term refers to m5D (5-methyldihydrouridine). In another embodiment, the term refers to m³Ψ (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

In some embodiments, chemical modifications to the ribonucleotides of the circular polyribonucleotide can enhance immune evasion. Modifications include, for example, end modifications, e.g., 5′-end modifications (phosphorylation (mono-, di- and tri-), conjugation, inverted linkages, etc.), 3′-end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified ribonucleotide bases can also include 5-methylcytidine and pseudouridine. In some embodiments, base modifications can modulate expression, immune response, stability, subcellular localization, to name a few functional effects, of the circular polyribonucleotide. In some embodiments, the modification includes a bi-orthogonal nucleotide, e.g., an unnatural base.

In some embodiments, sugar modifications (e.g., at the 2′ position or 4′ position) or replacement of the sugar one or more ribonucleotides of the circular polyribonucleotide can, as well as backbone modifications, include modification or replacement of the phosphodiester linkages. Non-limiting examples of circular polyribonucleotide include circular polyribonucleotide with modified backbones or non-natural internucleoside linkages, such as those modified or replaced of the phosphodiester linkages. Circular polyribonucleotides having modified backbones include, among others, those that do not have a phosphorus atom in the backbone. For the purposes of this application, and as sometimes referenced in the art, modified RNA that do not have a phosphorus atom in their internucleoside backbone can also be considered to be oligonucleosides. In particular embodiments, the circular polyribonucleotide will include ribonucleotides with a phosphorus atom in its internucleoside backbone.

Modified circular polyribonucleotide backbones can include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the circular polyribonucleotide can be negatively or positively charged.

The modified nucleotides, which can be incorporated into the circular polyribonucleotide, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the circular polyribonucleotide is expected to reduce the innate immune response through weaker binding/activation of cellular innate immune molecules.

In some embodiments, a modified nucleoside includes an α-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine). Other internucleoside linkages can include internucleoside linkages which do not contain a phosphorous atom.

In some embodiments, the circular polyribonucleotide can include one or more cytotoxic nucleosides. For example, cytotoxic nucleosides can be incorporated into circular polyribonucleotide, such as bifunctional modification. Cytotoxic nucleoside can include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((R,S)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

The circular polyribonucleotide can be uniformly modified along the entire length of the molecule. For example, one or more or all types of nucleotide (e.g., naturally-occurring nucleotides, purine or pyrimidine, or any one or more or all of A, G, U, C, I, pU) can be uniformly modified in the circular polyribonucleotide, or in a given predetermined sequence region thereof. In some embodiments, the circular polyribonucleotide includes a pseudouridine. In some embodiments, the circular polyribonucleotide includes an inosine, which can aid in the immune system characterizing the circular polyribonucleotide as endogenous versus viral RNA. The incorporation of inosine can also mediate improved RNA stability/reduced degradation.

In some embodiments, all nucleotides in the circular polyribonucleotide (or in a given sequence region thereof) are modified. In some embodiments, the modification can include an m6A, which can augment expression; an inosine, which can attenuate an immune response; pseudouridine, which can increase RNA stability, or translational readthrough (stop codon=coding potential), an m5C, which can increase stability; and a 2,2,7-trimethylguanosine, which aids subcellular translocation (e.g., nuclear localization).

Different sugar modifications, nucleotide modifications, and/or internucleoside linkages (e.g., backbone structures) can exist at various positions in the circular polyribonucleotide. One of ordinary skill in the art will appreciate that the nucleotide analogs or other modification(s) can be located at any position(s) of the circular polyribonucleotide, such that the function of the circular polyribonucleotide is not substantially decreased. A modification can also be a non-coding region modification. The circular polyribonucleotide can include from about 1% to about 100% modified nucleotides (either in relation to overall nucleotide content, or in relation to one or more types of nucleotide, i.e., any one or more of A, G, U, or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 950%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, the circular polyribonucleotide provided herein is a modified circular polyribonucleotide. For example, a completely modified circular polyribonucleotide comprises all or substantially all modified adenosine residues, all or substantially all modified uridine residues, all or substantially all modified guanine residues, all or substantially all modified cytidine residues, or any combination thereof. In some embodiments, the circular polyribonucleotide provided herein is a hybrid modified circular polyribonucleotide. A hybrid modified circular polyribonucleotide can have at least one modified nucleotide and can have a portion of contiguous unmodified nucleotides. This unmodified portion of the hybrid modified circular polyribonucleotide can have at least about 5, 10, 15, or 20 contiguous unmodified nucleotides, or any number therebetween. In some embodiments, the unmodified portion of the hybrid modified circular polyribonucleotide has at least about 30, 40, 40, 60, 70, 80, 90, 100, 110, 120, 130, 140, 150, 160, 180, 200, 220, 250, 280, 300, 320, 350, 380, 400, 420, 450, 500, 550, 600, 650, 700, 750, 800, 850, 900, or 1000 contiguous unmodified nucleotides, or any number therebetween. In some embodiments, the hybrid modified circular polyribonucleotide has 1, 2, 3, 4, 5, 6, 7, 8, 9, 10, or more unmodified portions. In some embodiments, the hybrid modified circular polyribonucleotide has at least 1, 2, 3, 4, 5, 6, 7, 8, 9, 10, 12, 15, 20, 30, 40, 50, 70, 80, 100, 120, 150, 200, 250, 300, 400, 500, 600, 700, 800, 900, 1000, or more modified nucleotides. In some embodiments, the hybrid modified circular polyribonucleotide has at least 1%, 2%, 5%, 7%, 8%, 10%, 12%, 15%, 18%, 20%, 25%, 30%, 35%, 40%, 45%, 50%, 55%, 60%, 65%, 70%, 80%, 90%, 95%, or 99% but less than 100% nucleotides that are modified. In some embodiments, the unmodified portion comprises a binding site. In some embodiments, the unmodified portion comprises a binding site configured to bind a protein, DNA, RNA, or a cell target. In some embodiments, the unmodified portion comprises an IRES.

In some embodiments, the hybrid modified circular polyribonucleotide has a lower immunogenicity than a corresponding unmodified circular polyribonucleotide. In some embodiments, the hybrid modified circular polyribonucleotide has an immunogenicity that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold lower than a corresponding unmodified circular polyribonucleotide. In some embodiments, the immunogenicity as described herein is assessed by the level of expression or signaling or activation of at least one of RIG-I, TLR-3, TLR-7, TLR-8, MDA-5, LGP-2, OAS, OASL, PKR, and IFN-beta. In some embodiments, the hybrid modified circular polyribonucleotide has a higher half-life than a corresponding unmodified circular polyribonucleotide. In some embodiments, the hybrid modified circular polyribonucleotide has a half-life that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold higher than a corresponding unmodified circular polyribonucleotide. In some embodiments, the half-life is measured by introducing the circular polyribonucleotide or the corresponding circular polyribonucleotide into a cell and measuring a level of the introduced circular polyribonucleotide or corresponding circular polyribonucleotide inside the cell.

In some embodiments, the hybrid modified circular polyribonucleotide comprises one or more expression sequences. In some embodiments, the one or more expression sequences of the hybrid modified circular polyribonucleotide has a translation efficiency similar to or higher than a corresponding unmodified circular polyribonucleotide. In some embodiments, the one or more expression sequences of the hybrid modified circular polyribonucleotide have a translation efficiency of that is at least about 0.7, 0.8, 0.9, 1.0, 1.1, 1.2, 1.3, 1.5, 1.6, 1.8, 2, 2.2, 2.5, 2.8, or 3 fold higher than a corresponding unmodified circular polyribonucleotide. In some embodiments, the one or more expression sequences of the hybrid modified circular polyribonucleotide have a higher translation efficiency than a corresponding circular polyribonucleotide having a portion comprising a modified nucleotide (e.g., the portion corresponds to the unmodified portion of the hybrid modified circular polyribonucleotide). In some embodiments, one or more expression sequences of the circular polyribonucleotide are configured to have a higher translation efficiency than a corresponding circular polyribonucleotide having a first portion comprising more than 10%, or at least 20%, 30%, 40%, 50%, 60%, 70%, 80%, 90%, or 100% modified nucleotides. In some embodiments, the one or more expression sequences of the hybrid modified circular polyribonucleotide has a translation efficiency that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold higher than a corresponding circular polyribonucleotide having a portion comprising a modified nucleotide (e.g., the portion corresponds to the unmodified portion of the hybrid modified circular polyribonucleotide). As described herein, in some embodiments, the translation efficiency is measured either in a cell comprising the circular polyribonucleotide or the corresponding circular polyribonucleotide, or in an in vitro translation system (e.g., rabbit reticulocyte lysate).

In some embodiments, the hybrid modified circular polyribonucleotide has a binding site that is unmodified, e.g., having no modified nucleotides. In some embodiments, the hybrid modified circular polyribonucleotide has a binding site configured to bind to a protein, DNA, RNA, or cell target that is unmodified, e.g., having no modified nucleotides. In some embodiments, the hybrid modified circular polyribonucleotide has an internal ribosome entry site (IRES) that is unmodified, e.g., having no modified nucleotides. In some embodiments, the hybrid modified circular polyribonucleotide has no more than 10% of the nucleotides in the binding site that are modified nucleotides. In some embodiments, the hybrid modified circular polyribonucleotide has no more than 10% of the nucleotides in the binding site configured to bind to a protein, DNA, RNA, or cell target that are modified nucleotides. In some embodiments, the hybrid modified circular polyribonucleotide has no more than 10% of the nucleotides in the internal ribosome entry site (IRES) that are modified nucleotides. In some embodiments, a hybrid modified circular polyribonucleotide has modified nucleotides throughout except the binding site. In some embodiments, a hybrid modified circular polyribonucleotide has modified nucleotides throughout except the binding site configured to bind a protein, DNA, RNA, or a cell target. In some embodiments, a hybrid modified circular polyribonucleotide has modified nucleotides throughout except the IRES element. In other embodiments, the hybrid modified circular polyribonucleotide has modified nucleotides throughout except the IRES element and one or more other portions. Without wishing to be bound by a certain theory, the unmodified IRES element renders the hybrid modified circular polyribonucleotide translation competent, e.g., having a translation efficiency for the one or more expression sequences that is similar to or higher than a corresponding circular polyribonucleotide that does not have any modified nucleotides.

In some embodiments, the hybrid modified circular polyribonucleotide has modified nucleotides, e.g., 5′ methylcytidine and pseudouridine, throughout the circular polyribonucleotide except the IRES element or a binding site configured to bind a protein, DNA, RNA, or a cell target. In these cases, the hybrid modified circular polyribonucleotide has a higher a lower immunogenicity as compared to a corresponding circular polyribonucleotide that does not comprise 5′ methylcytidine and pseudouridine. In some embodiments, the hybrid modified circular polyribonucleotide has an immunogenicity that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold lower than a corresponding unmodified circular polyribonucleotide. In some embodiments, the immunogenicity as described herein is assessed by expression or signaling or activation of at least one of RIG-I, TLR-3, TLR-7, TLR-8, MDA-5, LGP-2, OAS, OASL, PKR, and IFN-beta. In some embodiments, the hybrid modified circular polyribonucleotide has n higher half-life than a corresponding unmodified circular polyribonucleotide, e.g., a corresponding circular polyribonucleotide that does not comprise 5′ methylcytidine and pseudouridine. In some embodiments, the hybrid modified circular polyribonucleotide has a higher half-life that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold higher than a corresponding unmodified circular polyribonucleotide. In some embodiments, the half-life is measured by introducing the circular polyribonucleotide or the corresponding circular polyribonucleotide into a cell and measuring a level of the introduced circular polyribonucleotide or corresponding circular polyribonucleotide inside the cell.

In some cases, the hybrid modified circular polyribonucleotide as described herein has similar immunogenicity as compared to a corresponding circular polyribonucleotide that is otherwise the same but completely modified. For instance, a hybrid modified circular polyribonucleotide that has 5′ methylcytidine and pseudouridine throughout except its IRES element can have similar immunogenicity or lower immunogenicity as compared to a corresponding circular polyribonucleotide that is otherwise the same but has 5′ methylcytidine and pseudouridine throughout and no unmodified cytidine and uridine. In some embodiments, the hybrid modified circular polyribonucleotide that has 5′ methylcytidine and pseudouridine throughout except its IRES element has translation efficiency that is similar to or higher than the translation efficiency of a corresponding circular polyribonucleotide that is otherwise the same but has 5′ methylcytidine and pseudouridine throughout and no unmodified cytidine and uridine.

### Conjugation of Circular Polyribonucleotides

A circRNA of the disclosure can be conjugated, for example, to a chemical compound (e.g., a small molecule), an antibody or fragment thereof, a peptide, a protein, an aptamer, a drug, or a combination thereof. In some embodiments, a small molecule can be conjugated to a circRNA, thereby generating a circRNA comprising a small molecule.

A circRNA of the disclosure can comprise a conjugation moiety to facilitate conjugation. A conjugation moiety can be incorporated, for example, at an internal site of a circular polynucleotide, or at a 5′ end, 3′ end, or internal site of a linear polynucleotide. A conjugation moiety can be incorporated chemically or enzymatically. For example, a conjugation moiety can be incorporated during solid phase oligonucleotide synthesis, cotranscriptionally (e.g., with a tolerant RNA polymerase) or posttranscriptionally (e.g., with a RNA methyltransferase). A conjugation moiety can be a modified nucleotide or a nucleotide analog, e.g., bromodeoxyuridine. A conjugation moiety can comprise a reactive group or a functional group, e.g., an azide group or an alkyne group. A conjugation moiety can be capable of undergoing a chemoselective reaction. A conjugation moiety can be a hapten group, e.g., comprising digoxigenin, 2,4-dinitrophenyl, biotin, avidin, or selected from azoles, nitroaryl compounds, benzofurazans, triterpenes, ureas, thioureas, rotenones, oxazoles, thiazoles, coumarins, cyclolignans, heterobiaryl compounds, azoaryl compounds or benzodiazepines. A conjugation moiety can comprise a diarylethene photoswitch capable of undergoing reversible electrocyclic rearrangement. A conjugation moiety can comprise a nucleophile, a carbanion, and/or an α,β-unsaturated carbonyl compound.

A circRNA can be conjugated via a chemical reaction, e.g., using click chemistry, Staudinger ligation, Pd-catalyzed C—C bond formation (e.g., Suzuki-Miyaura reaction), Michael addition, olefin metathesis, or inverse electron demand Diels-Alder. Click chemistry can utilize pairs of functional groups that rapidly and selectively react (“click”) with each other in appropriate reaction conditions. Non-limiting click chemistry reactions include azide-alkyne cycloaddition, copper-catalyzed 1,3-dipolar azide-alkyne cycloaddition (CuAAC), strain-promoted Azide-Alkyne Click Chemistry reaction (SPAAC), and tetrazine-alkene Ligation.

Non-limiting examples of functionalized nucleotides include azide modified UTP analogs, 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 3′-Azido-2′,3′-ddATP, 5-Azido-PEG4-CTP, 5-DBCO-PEG4-CTP, N6-Azidohexyl-3′-dATP, 5-DBCO-PEG4-dCpG, and 5-azidopropyl-UTP. In some embodiments, a circRNA comprises at least one 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 5-Azido-PEG4-CTP, 5-DBCO-PEG4-CTP, or 5-azidopropyl-UTP.

A single modified nucleotide of choice (e.g., modified A, C, G, U, or T containing an azide at the 2′-position) can be incorporated site-specifically under optimized conditions (e.g., via solid-phase chemical synthesis). A plurality of nucleotides containing an azide at the 2′-position can be incorporated, for example, by substituting a nucleotide during an in vitro transcription reaction (e.g., substituting UTP for 5-azido-C3-UTP).

A circRNA conjugate can be generated using a copper-catalyzed click reaction, e.g., copper-catalyzed 1,3-dipolar azide-alkyne cycloaddition (CuAAC) of an alkyne-functionalized small molecule and an azide-functionalized polyribonucleic acid. A linear RNA can be conjugated with a small molecule. For example, a linear RNA can be modified at its 3′-end by a poly(A) polymerase with an azido-derivatized nucleotide. The azide can be conjugated to a small molecule via copper-catalyzed or strain-promoted azide-alkyne click reaction, and the linear RNA can be circularized.

A circRNA conjugate can be generated using a Staudinger reaction. For example, a circular RNA comprising an azide-functionalized nucleotide can be conjugated with an alkyne-functionalized small molecule in the presence of triphenylphosphine-3,3′,3″-trisulfonic acid (TPPTS).

A circRNA conjugate can be generated using a Suzuki-Miyaura reaction. For example, a circRNA comprising a halogenated nucleotide analog can be subjected to Suzuki-Miyaura reaction in the presence of a cognate reactive partner. A circRNA comprising 5-Iodouridine triphosphate (IUTP), for example, can be used in a catalytic system with Pd(OAc)₂ and 2-aminopyrimidine-4,6-diol (ADHP) or dimethylamino-substituted ADHP (DMADHP) to functionalize iodouridine-labeled circRNA in the presence of various boronic acid and ester substrates. In another example, a circRNA comprising 8-bromoguanosine can be reacted with arylboronic acids in the presence of a catalytic system made of Pd(OAc)₂ and a water-soluble triphenylphosphan-3,3′,3″-trisulfonate ligand.

A circRNA conjugate can be generated using Michael addition, for example, via reaction of an an electron-rich Michael Donor with an α,β-unsaturated compound (Michael Acceptor).

### Structure

In some embodiments, the circular polyribonucleotide comprises a higher order structure, e.g., a secondary or tertiary structure. In some embodiments, complementary segments of the circular polyribonucleotide fold itself into a double stranded segment, held together with hydrogen bonds between pairs, e.g., A-U and C-G. In some embodiments, helices, also known as stems, are formed intra-molecularly, having a double-stranded segment connected to an end loop. In some embodiments, the circular polyribonucleotide has at least one segment with a quasi-double-stranded secondary structure. In some embodiments, a segment having a quasi-double-stranded secondary structure has at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides. In some embodiments, the circular polyribonucleotide has one or more segments (e.g., 2, 3, 4, 5, 6, or more) having a quasi-double-stranded secondary structure. In some embodiments, the segments are separated by 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides.

There are 16 possible base-pairings, however of these, six (AU, GU, GC, UA, UG, CG) can form actual base-pairs. The rest are called mismatches and occur at very low frequencies in helices. In some embodiments, the structure of the circular polyribonucleotide cannot easily be disrupted without impact on its function and lethal consequences, which provide a selection to maintain the secondary structure. In some embodiments, the primary structure of the stems (i.e., their nucleotide sequence) can still vary, while still maintaining helical regions. The nature of the bases is secondary to the higher structure, and substitutions are possible as long as they preserve the secondary structure. In some embodiments, the circular polyribonucleotide has a quasi-helical structure. In some embodiments, the circular polyribonucleotide has at least one segment with a quasi-helical structure. In some embodiments, a segment having a quasi-helical structure has at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides. In some embodiments, the circular polyribonucleotide has one or more segments (e.g., 2, 3, 4, 5, 6, or more) having a quasi-helical structure. In some embodiments, the segments are separated by 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides. In some embodiments, the circular polyribonucleotide includes at least one of a U-rich or A-rich sequence or a combination thereof. In some embodiments, the U-rich and/or A-rich sequences are arranged in a manner that would produce a triple quasi-helix structure. In some embodiments, the circular polyribonucleotide has a double quasi-helical structure. In some embodiments, the circular polyribonucleotide has one or more segments (e.g., 2, 3, 4, 5, 6, or more) having a double quasi-helical structure. In some embodiments, the circular polyribonucleotide includes at least one of a C-rich and/or G-rich sequence. In some embodiments, the C-rich and/or G-rich sequences are arranged in a manner that would produce triple quasi-helix structure. In some embodiments, the circular polyribonucleotide has an intramolecular triple quasi-helix structure that aids in stabilization.

In some embodiments, the circular polyribonucleotide has two quasi-helical structure (e.g., separated by a phosphodiester linkage), such that their terminal base pairs stack, and the quasi-helical structures become colinear, resulting in a “coaxially stacked” substructure.

In some embodiments, the circular polyribonucleotide has at least one miRNA binding site, at least one lncRNA binding site, and/or at least one tRNA motif.

### Circularization

In some embodiments, a linear circular polyribonucleotide can be cyclized or concatemerized. In some embodiments, the linear circular polyribonucleotide can be cyclized in vitro prior to formulation and/or delivery. In some embodiments, linear circular polyribonucleotides can be cyclized within a cell.

### Extracellular Circularization

In some embodiments, a linear circular polyribonucleotide is cyclized, or concatemerized using a chemical method to form a circular polyribonucleotide. In some chemical methods, the 5′-end and the 3′-end of the nucleic acid (e.g., a linear circular polyribonucleotide) includes chemically reactive groups that, when close together, can form a new covalent linkage between the 5′-end and the 3′-end of the molecule. The 5′-end can contain an NHS-ester reactive group and the 3′-end can contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′- or 3′-amide bond.

In some embodiments, a DNA or RNA ligase may be used to enzymatically link a 5′-phosphorylated nucleic acid molecule (e.g., a linear circular polyribonucleotide) to the 3′-hydroxyl group of a nucleic acid (e.g., a linear nucleic acid) forming a new phosphorodiester linkage. In an example reaction, a linear circular polyribonucleotide is incubated at 37° C. for 1 hour with 1-10 units of T4 RNA ligase (New England Biolabs, Ipswich, Mass.) according to the manufacturer's protocol. The ligation reaction may occur in the presence of a linear nucleic acid capable of base-pairing with both the 5′- and 3′-region in juxtaposition to assist the enzymatic ligation reaction. In some embodiments, the ligation is splint ligation. For example, a splint ligase, like SplintR® ligase, can be used for splint ligation. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide.

In some embodiments, a DNA or RNA ligase can be used in the synthesis of the circular polynucleotides. As a non-limiting example, the ligase can be a circ ligase or circular ligase.

In some embodiments, either the 5′- or 3′-end of the linear circular polyribonucleotide can encode a ligase ribozyme sequence such that during in vitro transcription, the resultant linear circular polyribonucleotide includes an active ribozyme sequence capable of ligating the 5′-end of the linear circular polyribonucleotide to the 3′-end of the linear circular polyribonucleotide. The ligase ribozyme can be derived from the Group I Intron, Hepatitis Delta Virus, Hairpin ribozyme or can be selected by SELEX (systematic evolution of ligands by exponential enrichment). The ribozyme ligase reaction can take 1 to 24 hours at temperatures between 0 and 37° C.

In some embodiments, a linear circular polyribonucleotide can be cyclized or concatermerized by using at least one non-nucleic acid moiety. In one aspect, the at least one non-nucleic acid moiety can react with regions or features near the 5′-terminus and/or near the 3′-terminus of the linear circular polyribonucleotide in order to cyclize or concatermerize the linear circular polyribonucleotide. In another aspect, the at least one non-nucleic acid moiety can be located in or linked to or near the 5′-terminus and/or the 3′-terminus of the linear circular polyribonucleotide. The non-nucleic acid moieties contemplated can be homologous or heterologous. As a non-limiting example, the non-nucleic acid moiety can be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety can be an oligonucleotide or a peptide moiety, such as an aptamer or a non-nucleic acid linker as described herein.

In some embodiments, a linear circular polyribonucleotide can be cyclized or concatermerized due to a non-nucleic acid moiety that causes an attraction between atoms, molecular surfaces at, near or linked to the 5′- and 3′-ends of the linear circular polyribonucleotide. As a non-limiting example, one or more linear circular polyribonucleotides can be cyclized or concantermized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In some embodiments, the linear circular polyribonucleotide can comprise a ribozyme RNA sequence near the 5′-terminus and near the 3′-terminus. The ribozyme RNA sequence can covalently link to a peptide when the sequence is exposed to the remainder of the ribozyme. In one aspect, the peptides covalently linked to the ribozyme RNA sequence near the 5′-terminus and the 3′-terminus can associate with each other causing a linear circular polyribonucleotide to cyclize or concatemerize. In another aspect, the peptides covalently linked to the ribozyme RNA near the 5′-terminus and the 3′-terminus can cause the linear primary construct or linear mRNA to cyclize or concatemerize after being subjected to ligation using various methods known in the art such as, but not limited to, protein ligation. Non-limiting examples of ribozymes for use in the linear primary constructs or linear RNA of the present invention or a non-exhaustive listing of methods to incorporate and/or covalently link peptides are described in US patent application No. US20030082768, the contents of which is here in incorporated by reference in its entirety.

In some embodiments, the linear circular polyribonucleotide can include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear circular polyribonucleotide into a 5′ monophosphate can occur by a two-step reaction comprising: (a) contacting the 5′ nucleotide of the linear circular polyribonucleotide with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%.

In some embodiment, the circular polyribonucleotide includes at least one splicing element. Exemplary splicing elements are described in paragraphs [0270]-[0275] of WO2019/118919, which is hereby incorporated by reference in its entirety.

### Other Circularization Methods

In some embodiments, linear circular polyribonucleotides can include complementary sequences, including either repetitive or nonrepetitive nucleic acid sequences within individual introns or across flanking introns. Repetitive nucleic acid sequences are sequences that occur within a segment of the circular polyribonucleotide. In some embodiments, the circular polyribonucleotide includes a repetitive nucleic acid sequence. In some embodiments, the repetitive nucleotide sequence includes poly CA or poly UG sequences. In some embodiments, the circular polyribonucleotide includes at least one repetitive nucleic acid sequence that hybridizes to a complementary repetitive nucleic acid sequence in another segment of the circular polyribonucleotide, with the hybridized segment forming an internal double strand. In some embodiments, repetitive nucleic acid sequences and complementary repetitive nucleic acid sequences from two separate circular polyribonucleotides hybridize to generate a single circularized polyribonucleotide, with the hybridized segments forming internal double strands. In some embodiments, the complementary sequences are found at the 5′- and 3′-ends of the linear circular polyribonucleotides. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the circular polyribonucleotide. Such methods may include, but are not limited to click chemistry (e.g., alkyne and azide based methods, or clickable bases), olefin metathesis, phosphoramidate ligation, hemiaminal-imine crosslinking, base modification, and any combination thereof.

In some embodiments, enzymatic methods of circularization may be used to generate the circular polyribonucleotide. In some embodiments, a ligation enzyme, e.g., DNA or RNA ligase, may be used to generate a template of the circular polyribonuclease or complement, a complementary strand of the circular polyribonuclease, or the circular polyribonuclease.

Circularization of the circular polyribonucleotide may be accomplished by methods known in the art, for example, those described in “RNA circularization strategies in vivo and in vitro” by Petkovic and Muller from Nucleic Acids Res, 2015, 43(4): 2454-2465, and “In vitro circularization of RNA” by Muller and Appel, from RNA Biol, 2017, 14(8):1018-1027.

The circular polyribonucleotide may encode a sequence and/or motifs useful for replication. Exemplary replication elements include binding sites for RNA polymerase. Other types of replication elements are described in paragraphs [0280]-[0286] of WO2019/118919, which is hereby incorporated by reference in its entirety. In some embodiments, the circular polyribonucleotide as disclosed herein lacks a replication element, e.g., lacks an RNA-dependent RNA polymerase binding site.

In some embodiments, the circular polyribonucleotide lacks a poly-A sequence and a replication element.

### Methods of Production

In some embodiments, the circular polyribonucleotide includes a deoxyribonucleic acid sequence that is non-naturally occurring and can be produced using recombinant technology (e.g., derived in vitro using a DNA plasmid), chemical synthesis, or a combination thereof.

It is within the scope of the disclosure that a DNA molecule used to produce an RNA circle can comprise a DNA sequence of a naturally-occurring original nucleic acid sequence, a modified version thereof, or a DNA sequence encoding a synthetic polypeptide not normally found in nature (e.g., chimeric molecules or fusion proteins, such as fusion proteins comprising multiple antigens and/or epitopes). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

The circular polyribonucleotide may be prepared according to any available technique including, but not limited to chemical synthesis and enzymatic synthesis. In some embodiments, a linear primary construct or linear mRNA may be cyclized, or concatemerized to create a circular polyribonucleotide described herein. The mechanism of cyclization or concatemerization may occur through methods such as, but not limited to, chemical, enzymatic, splint ligation), or ribozyme catalyzed methods. The newly formed 5′-/3′-linkage may be an intramolecular linkage or an intermolecular linkage.

Methods of making the circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

Various methods of synthesizing circular polyribonucleotides are also described in the art (see, e.g., U.S. Pat. Nos. 6,210,931, 5,773,244, 5,766,903, 5,712,128, 5,426,180, US Publication No. US20100137407, International Publication No. WO1992001813 and International Publication No. WO2010084371; the contents of each of which are herein incorporated by reference in their entireties).

In some embodiments, the circular polyribonucleotides is purified, e.g., free ribonucleic acids, linear or nicked RNA, DNA, proteins, etc are removed. In some embodiments, the circular polyribonucleotides may be purified by any known method commonly used in the art. Examples of nonlimiting purification methods include, column chromatography, gel excision, size exclusion, etc.

### Applications

A parenteral delivery system described herein comprises a circular polyribonucleotide and a parenterally acceptable diluent, wherein the circular polyribonucleotide comprises a sequence that binds a target. The parenteral delivery system can be a delivery system free of any carrier. The parenteral delivery system further comprise a carrier. The invention also contemplates a method of in vivo delivery of a circular polyribonucleotide comprising parenterally administering the circular polyribonucleotide to a subject, wherein the circular polyribonucleotide comprises a sequence that binds a target. The in vivo delivery of a circular polyribonucleotide can also comprise a method of in vivo delivery of a circular polyribonucleotide to a cell or tissue of a subject comprising parenterally administering to the cell or tissue the circular polyribonucleotide, wherein the circular polyribonucleotide comprises a sequence that binds a target. The circular polyribonucleotide can be in a composition. Parenteral administration can comprise intramuscular administration, intravenous administration, ophthalmic administration, or topical administration. Circular polyribonucleotides described herein can be administered to a cell, tissue or subject in need thereof, e.g., to modulate cellular function or a cellular process, e.g., gene expression in the cell, tissue or subject. The invention also contemplates methods of modulating cellular function or a cellular process, e.g., gene expression, comprising administering to a cell, tissue or subject in need thereof a circular polyribonucleotide described herein. The administered circular polyribonucleotides can be modified circular polyribonucleotides. In some embodiments, the administered circular polyribonucleotides are completely modified circular polyribonucleotides. In some embodiments, the administered circular polyribonucleotides are hybrid modified circular polyribonucleotides. In other embodiments, the administered circular polyribonucleotides are unmodified circular polyribonucleotides.

A parenteral delivery system described herein comprises a circular polyribonucleotide and a parenterally acceptable diluent, wherein the circular polyribonucleotide is a translation incompetent circular polyribonucleotide and comprises a sequence that binds a target. The parenteral delivery system can be a delivery system free of any carrier. The parenteral delivery system further comprise a carrier. The invention also provides a method of in vivo delivery of a circular polyribonucleotide comprising parenterally administering the circular polyribonucleotide to a subject, wherein the circular polyribonucleotide is a translation incompetent circular polyribonucleotide and comprises a sequence that binds a target. The in vivo delivery of a circular polyribonucleotide can also comprise a method of in vivo delivery of a circular polyribonucleotide to a cell or tissue of a subject comprising parenterally administering to the cell or tissue the circular polyribonucleotide, wherein the circular polyribonucleotide is a translation incompetent circular polyribonucleotide and comprises a sequence that binds a target. The circular polyribonucleotide can be in a composition. Parenteral administration can comprise intramuscular administration, intravenous administration, ophthalmic administration, or topical administration. Circular polyribonucleotides described herein can be administered to a cell, tissue or subject in need thereof, e.g., to modulate cellular function or a cellular process, e.g., gene expression in the cell, tissue or subject. The invention also contemplates methods of modulating cellular function or a cellular process, e.g., gene expression, comprising administering to a cell, tissue or subject in need thereof a circular polyribonucleotide described herein. The administered circular polyribonucleotides can be modified circular polyribonucleotides. In some embodiments, the administered circular polyribonucleotides are completely modified circular polyribonucleotides. In some embodiments, the administered circular polyribonucleotides are hybrid modified circular polyribonucleotides. In other embodiments, the administered circular polyribonucleotides are unmodified circular polyribonucleotides.

In some embodiments, the nucleic acid delivery system as disclosed herein is used as a medicament or a pharmaceutical. A nucleic acid delivery system as disclosed herein can be used in a method of treatment of a human or animal body by therapy. A nucleic acid delivery system as disclosed herein can be used in the manufacture of a medicament or a pharmaceutical. A nucleic acid delivery system as disclosed herein can be used in the manufacture of a medicament or a pharmaceutical for treating a human or animal body by therapy.

In some embodiments, the parenteral delivery system as disclosed herein is used as a medicament or a pharmaceutical. A parenteral delivery system as disclosed herein can be used in a method of treatment of a human or animal body by therapy. A parenteral delivery system as disclosed herein can be used in the manufacture of a medicament or a pharmaceutical. A parenteral delivery system as disclosed herein can be used in the manufacture of a medicament or a pharmaceutical for treating a human or animal body by therapy.

In some embodiments, the circular polyribonucleotide lacks a poly-A sequence, lacks a replication element, lacks a free 3′ end, or lacks an RNA polymerase recognition motif, or any combination thereof. In some embodiments, the circular polyribonucleotide lacks a poly-A sequence. In some embodiments, the circular polyribonucleotide lacks a replication element. In some embodiments, the circular polyribonucleotide lacks a free 3′ end. In some embodiments, the circular polyribonucleotide lacks an RNA polymerase recognition motif. In some embodiments, the circular polyribonucleotide is a translation incompetent circular polyribonucleotide.

In some embodiments, the circular polyribonucleotide further comprises an expression sequence. In some embodiments, the circular polyribonucleotide comprises a termination element or an IRES, or the combination thereof.

All references and publications cited herein are hereby incorporated by reference.

The following examples are provided to further illustrate some embodiments of the present invention, for example using model elements, but are not intended to limit the scope of the invention; it will be understood by their exemplary nature that other procedures, methodologies, or techniques known to those skilled in the art can alternatively be used.

### Numbered Embodiments #1

[1] A parenteral nucleic acid delivery system comprising a circular polyribonucleotide and a parenterally acceptable diluent, wherein the circular polyribonucleotide is a translation incompetent circular polyribonucleotide and comprises a sequence that binds a target.
[2] The parenteral nucleic acid delivery system of embodiment [1], wherein the delivery system is free of any carrier.
[3] A method of in vivo delivery of a circular polyribonucleotide comprising parenterally administering the circular polyribonucleotide to a subject, wherein the circular polyribonucleotide is a translation incompetent circular polyribonucleotide and comprises a sequence that binds a target.
[4] The method of embodiment [3], wherein the circular polyribonucleotide is in an amount effective to elicit a biological response in the subject.
[5] The method of embodiment [3], wherein the circular polyribonucleotide is in an amount effective to have a biological effect on a cell or tissue in the subject.
[6] A method of in vivo delivery of a circular polyribonucleotide to a cell or tissue of a subject comprising parenterally administering to the cell or tissue the circular polyribonucleotide, wherein the circular polyribonucleotide is a translation incompetent circular polyribonucleotide and comprises a sequence that binds a target.
[7] A method of delivering a composition to a subject, comprising administering the composition parenterally, to the subject, wherein the composition comprises a circular polyribonucleotide that is a translation incompetent circular polyribonucleotide and a sequence that binds a target.
[8] A method of in vivo delivery of a composition to a cell or tissue of a subject, comprising administering the composition parenterally to the cell or tissue, wherein the composition comprises a circular polyribonucleotide that is a translation incompetent circular polyribonucleotide and a sequence that binds a target.
[9] The method of any one of embodiments [3]-[6], wherein the circular polyribonucleotide is administered in a composition.
[10] The method of any one of embodiments [3]-[8], wherein parenteral administration is intravenously, intramuscularly, ophthalmically or topically.
[11] The method of any one of embodiments [7-[10], wherein the composition is a pharmaceutical composition further comprising a pharmaceutically acceptable excipient.
[12] The method of any one of embodiments [7]-[10], wherein the composition comprises a carrier.
[13] The method any one of embodiments [7]-[10], wherein the composition comprises a parenterally acceptable diluent and is free of any carrier.
[14] The system or method of any of the preceding embodiments, wherein the circular polyribonucleotide forms a complex with the target and the circular polyribonucleotide or the target is detectable at least 5 days after delivery.
[15] The system or method of any of the preceding embodiments, wherein the target is selected from the group consisting of a nucleic acid molecule, a small molecule, a protein, a carbohydrate, and a lipid.
[16] The system or method of embodiment [15], wherein the small molecule is an organic compound having a molecular weight of no more than 900 daltons and modulates a cellular process.
[17] The system or method of embodiment [16], wherein the small molecule is a drug.
[18] The system or method of embodiment [16], wherein the small molecule is a fluorophore.
[19] The system or method of embodiment [16], wherein the small molecule is a metabolite.
[20] The system or method of any of embodiments [1]-[19], wherein the target is a gene regulation protein.
[21] The system or method of any of embodiment [20], wherein the gene regulation protein is a transcription factor.
[22] The system or method of embodiment [15], wherein the nucleic acid molecule is a DNA molecule or an RNA molecule.
[23] The system or method of any one of embodiments [14]-[22], wherein the complex modulates gene expression.
[24] The system or method of any one of embodiments [14]-[23], wherein the complex modulates directed transcription of a DNA molecule, epigenetic remodeling of a DNA molecule, or degradation of a DNA molecule.
[25] The system or method of any one of embodiments [14]-[24] wherein the complex modulates degradation of the target, translocation of the target, or target signal transduction.
[26] The system or method of any one of embodiments [23]-[25], wherein the gene expression is associated with pathogenesis of a disease or condition.
[27] The system or method of any one of embodiments [14]-[26], wherein the circular polyribonucleotide of the complex or the target of the complex is detectable at least 7, 8, 9, or 10 days after delivery.
[28] The system or method of any one of the preceding embodiments, wherein the translation incompetent circular polyribonucleotide is present at least five days after delivery.
[29] The system or method of any one of the preceding embodiments, wherein the translation incompetent circular polyribonucleotide is present at least 6, 7, 8, 9, or 10 days after delivery.
[30] The system or method of any one of the preceding embodiments, wherein the translation incompetent circular polyribonucleotide is an unmodified translation incompetent circular polyribonucleotide.
[31] The system or method of any one of the preceding embodiments, wherein the translation incompetent circular polyribonucleotide has a quasi-double-stranded secondary structure.
[32] The system or method of any one of the preceding embodiments, wherein the sequence is an aptamer sequence that has a secondary structure that binds the target.
[33] The system or method of any one of the preceding embodiments, wherein the aptamer sequence further has a tertiary structure that binds the target.
[34] The method of any one of the preceding embodiments, wherein the cell is a eukaryotic cell.
[35] The method of embodiment [34], wherein the eukaryotic cell is an animal cell.
[36] The method of embodiment [34], wherein the eukaryotic cells is a pet cell.
[37] The method of embodiment [34], wherein the eukaryotic cell is a mammalian cell.
[38] The method of embodiment [34], wherein the eukaryotic cell is a human cell.
[39] The method of embodiment [34], wherein the eukaryotic cell is a livestock cell.

### Numbered Embodiments #2

[1] A parenteral nucleic acid delivery system comprising a circular polyribonucleotide and a parenterally acceptable diluent, wherein the circular polyribonucleotide comprises a sequence that binds a target.
[2] The parenteral nucleic acid delivery system of embodiment [1], wherein the delivery system is free of any carrier.
[3] The parenteral nucleic acid delivery system of embodiment [1], wherein the delivery system comprises a carrier.
[4] A method of in vivo delivery of a circular polyribonucleotide comprising parenterally administering the circular polyribonucleotide to a subject, wherein the circular polyribonucleotide comprises a sequence that binds a target.
[5] The method of embodiment [4], wherein the circular polyribonucleotide is in an amount effective to elicit a biological response in the subject.
[6] The method of embodiment [4], wherein the circular polyribonucleotide is in an amount effective to have a biological effect on a cell or tissue in the subject.
[7] A method of in vivo delivery of a circular polyribonucleotide to a cell or tissue of a subject comprising parenterally administering to the cell or tissue the circular polyribonucleotide, wherein the circular polyribonucleotide comprises a sequence that binds a target.
[8] A method of delivering a composition to a subject, comprising administering the composition parenterally, to the subject, wherein the composition comprises a circular polyribonucleotide comprising a sequence that binds a target.
[9] A method of in vivo delivery of a composition to a cell or tissue of a subject, comprising administering the composition parenterally to the cell or tissue, wherein the composition comprises a circular polyribonucleotide comprising a sequence that binds a target.
[10] The method of any one of embodiments [4]-[6], wherein the circular polyribonucleotide is administered in a composition.
[11] The method of any one of embodiments [4]-[9], wherein parenteral administration is intravenously, intramuscularly, ophthalmically or topically.
[12] The method of any one of embodiments [8]-[11], wherein the composition is a pharmaceutical composition further comprising a pharmaceutically acceptable excipient.
[13] The method of any one of embodiments [8]-[11], wherein the composition comprises a carrier.
[14] The method any one of embodiments [8]-[11], wherein the composition comprises a parenterally acceptable diluent and is free of any carrier.
[15] The system or method of any of the preceding embodiments, wherein the circular polyribonucleotide forms a complex with the target and the circular polyribonucleotide or the target is detectable at least 5 days after delivery.
[16] The system or method of any of the preceding embodiments, wherein the target is selected from the group consisting of a nucleic acid molecule, a small molecule, a protein, a carbohydrate, and a lipid.
[17] The system or method of embodiment [16], wherein the small molecule is an organic compound having a molecular weight of no more than 900 daltons and modulates a cellular process.
[18] The system or method of embodiment [17], wherein the small molecule is a drug.
[19] The system or method of embodiment [17], wherein the small molecule is a fluorophore.
[20] The system or method of embodiment [17], wherein the small molecule is a metabolite.
[21] The system or method of any of embodiments [1]-[20], wherein the target is a gene regulation protein.
[22] The system or method of any of embodiment [21], wherein the gene regulation protein is a transcription factor.
[23] The system or method of embodiment [16], wherein the nucleic acid molecule is a DNA molecule or an RNA molecule.
[24] The system or method of any one of embodiments [15]-[23], wherein the complex modulates gene expression.
[25] The system or method of any one of embodiments [15]-[24], wherein the complex modulates directed transcription of a DNA molecule, epigenetic remodeling of a DNA molecule, or degradation of a DNA molecule.
[26] The system or method of any one of embodiments [15]-[25] wherein the complex modulates degradation of the target, translocation of the target, or target signal transduction.
[27] The system or method of any one of embodiments [24]-[26], wherein the gene expression is associated with pathogenesis of a disease or condition.
[28] The system or method of any one of embodiments [15]-[27], wherein the circular polyribonucleotide of the complex or the target of the complex is detectable at least 7, 8, 9, or 10 days after delivery.
[29] The system or method of any one of the preceding embodiments, wherein the circular polyribonucleotide is present at least five days after delivery.
[30] The system or method of any one of the preceding embodiments, wherein the circular polyribonucleotide is present at least 6, 7, 8, 9, or 10 days after delivery.
[31] The system or method of any one of the preceding embodiments, wherein the circular polyribonucleotide is an unmodified circular polyribonucleotide.
[32] The system or method of any one of the preceding embodiments, wherein the circular polyribonucleotide has a quasi-double-stranded secondary structure.
[33] The system or method of any one of the preceding embodiments, wherein the sequence is an aptamer sequence that has a secondary structure that binds the target.
[34] The system or method of any one of the preceding embodiments, wherein the aptamer sequence further has a tertiary structure that binds the target.
[35] The method of any one of the preceding embodiments, wherein the cell is a eukaryotic cell.
[36] The method of embodiment [35], wherein the eukaryotic cell is an animal cell.
[37] The method of embodiment [35], wherein the eukaryotic cells is a pet cell.
[38] The method of embodiment [35], wherein the eukaryotic cell is a mammalian cell.
[39] The method of embodiment [35], wherein the eukaryotic cell is a human cell.
[40] The method of embodiment [35], wherein the eukaryotic cell is a livestock cell.
[41] The system or method of any one of the preceding embodiments, wherein the circular polyribonucleotide lacks a poly-A sequence, lacks a replication element, lacks a free 3′ end, or lacks an RNA polymerase recognition motif, or any combination thereof.
[42] The system or method of any one of the preceding embodiments, wherein the circular polyribonucleotide is a translation incompetent circular polyribonucleotide.
[43] The system or method of any one of the embodiments [1]-[42], wherein the circular polyribonucleotide further comprises an expression sequence.
[44] The system or method of embodiment [43], wherein the circular polyribonucleotide comprises a termination element or an IRES, or the combination thereof.

### EXAMPLES

### Example 1: Circular RNA Delivered Intracellularly in the Absence of Carrier (Naked Delivery)

This Example demonstrates functional circular RNA intracellular delivery in cells in the absence of carrier.

Active mango RNA aptamers fluoresce when bound by TO-1 biotin dye. As shown in the following Example, functional circular RNA containing a mango RNA aptamer bound to TO-1 biotin entered the cell in a carrier-independent manner.

Circular RNA was designed to include the mango RNA small molecule binding aptamer sites and a stabilizing stem: 5′-AATAGCCG GUCUACGGCC AUACCACCCU GAACGCGCCC GAUCUCGUCU GAUCUCGGAAGCUAAGCAGG GUCGGGCCUG GUUAGUACUU GGAUGGGAGA CCGCCUGGGAAUACCGGGUG CUGUAGGCGU CGACUUGCCA UGUGUAUGUG GGUACGAAGGAAGGAUUGGU AUGUGGUAUA UUCGUACCCA CAUACUCUGA UGAUCCUUCG GGAUCAUUCA UGGCAA CGGCTATT-3′ (SEQ ID NO: 1), as well as circularization sequences (“circular RNA aptamer”).

Unmodified linear RNA was synthesized by in vitro transcription using T7 RNA polymerase from a DNA segment comprising the mango RNA motif, stems and circularization sequences. Transcribed RNA was purified with an RNA cleanup kit (New England Biolabs, T2050), treated with RNA 5′-phosphohydrolase (RppH, New England Biolabs, M0356) following the manufacturer's instructions, and purified again with the RNA purification column. RppH treated RNA was circularized using a splint DNA and T4 RNA ligase. The circular RNA aptamer was urea-PAGE purified, eluted in a buffer, ethanol precipitated and resuspended in RNase free water. RNA quality is assessed by Urea-PAGE or through automated electrophoresis.

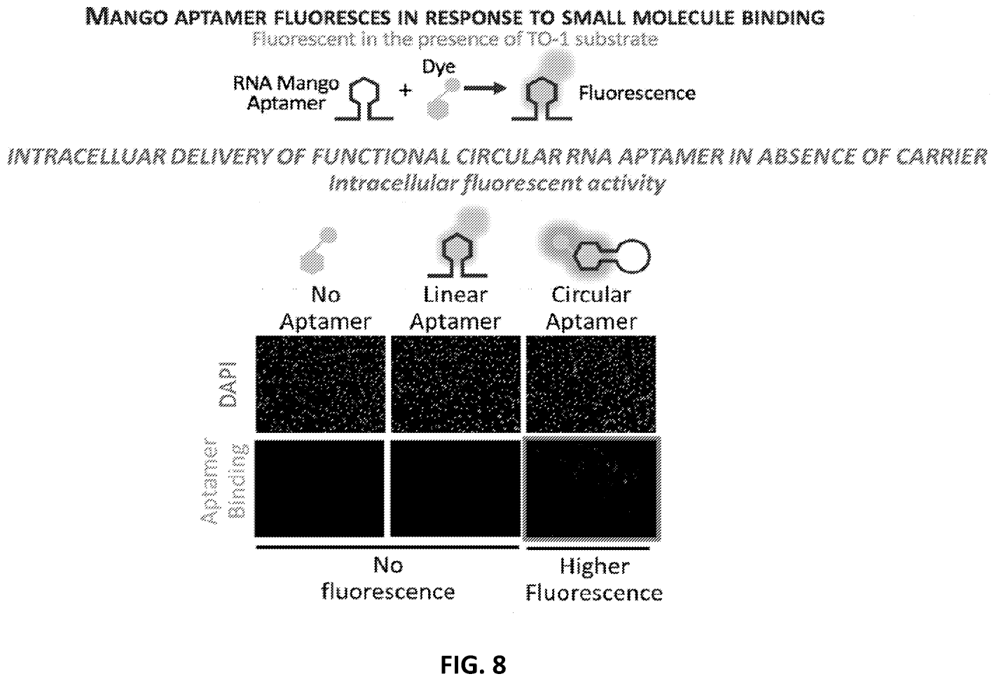

Circular RNA aptamer binding to TO-1 biotin was evaluated in vitro in BJ fibroblast cells, using fluorescent microscopy. When TO-1 biotin was bound to RNA, it enhanced its fluorescence more than 100-fold. Linear or circular RNA aptamers (50 nM) in a buffer, in the absence of any transfection reagent, were added to BJ fibroblasts. A no RNA control used buffer only. Cultures were treated with TO-1 biotin and fluorescence was analyzed after 6 hours. As shown in FIG. 8, fluorescence was detected in cells treated with the circular RNA aptamer, but not with the linear RNA aptamer or buffer alone.

This Example demonstrated that circular RNA was delivered intracellularly in the absence of carrier (naked).

### Example 2: Circular RNA-Mediated Delivery Directly into Specific Cell Types in the Absence of a Carrier (Naked Delivery)

This Example demonstrates circular RNA-mediated delivery directly into specific cell types in the absence of a carrier.

As shown in the following Example, circular RNA containing aptamer sequences within the circular RNA (where the RNA aptamer sequences target therapeutically-relevant proteins on a target cell) entered cells in a carrier-independent manner.

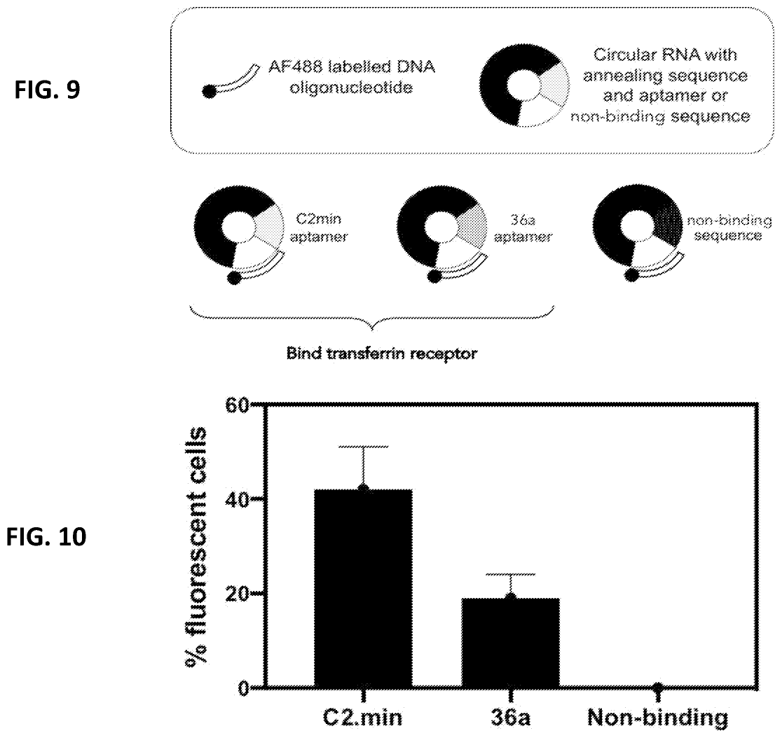

Circular RNAs were designed to include either an C2 min aptamer sequence known to bind competitively to the human transferrin receptor (5′-GGG GGA UCA AUC CAA GGG ACC CGG AAA CGC UCC CUU ACA CCC C-3′ (SEQ ID NO: 2)); or, a 36a (5′-GGG UGA AUG GUU CUA CGA UAA ACG UUA AUG ACC AGC UUA UGG CUG GCA GUU CCU AUA GCA CCC-3′ (SEQ ID NO: 3)) aptamer sequence known to bind non-competitively to the human transferrin receptor. Circular RNAs were designed to include a spacer region for hybridization of a fluorescent single stranded DNA oligonucleotide for visualization. A control circular RNA including an aptamer sequence that is predicted not to bind to human transferrin receptor was also used. A schematic of these circular RNAs is shown in FIG. 9.

Circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA (5′-TGT TGT GTC TTG GTT GGT-3′ (SEQ ID NO: 4) or 5′-TGT TGT GTG TTG GTT GGT-3′ (SEQ ID NO: 5)) and T4 RNA ligase. Circular RNA was urea-PAGE purified, eluted in a buffer, ethanol precipitated and resuspended in RNase storage solution (ThermoFisher Scientific, cat #AM7000).

A short single-stranded DNA oligonucleotide with AlexaFluor488 was used to label the aptamer for intracellular visualization. The fluorescent ssDNA oligonucleotide was added at 3× molar excess over the circular RNA, incubated at 60° C. for 10 minutes followed by a 20 minute incubation at room temperature. RNA buffer was exchanged to PBS using a Microbiospin column (Biorad).

Circular RNA annealed with the AlexaFluor488-DNA oligonucleotide was added to HeLa cells in the absence of carrier. Cells were imaged using a cell imager.

Circular RNA binding to human transferrin was evaluated by fluorescent microscopy. AlexaFluor488 activity was detected inside HeLa cells as punctate fluorescent signals when C2 min and 36a aptamers were contained in the circular RNA (FIG. 10). In contrast, no fluorescent signal was observed for the control circular RNA containing a non-binding aptamer sequence. This indicates aptamer sequences contained within the circular RNA were responsible for internalization via transferrin receptor binding.

This Example demonstrated that RNA aptamer sequences contained within circular RNA bind target proteins and that the circle RNA was delivered intracellularly into mammalian cells in the absence of carrier (naked) via interaction with the specific surface receptor.

### Example 3: Circular RNA-Mediated Delivery Directly into Specific Cell Types in the Absence of a Carrier (Naked Delivery)

This Example describes circular RNA-mediated delivery directly into specific cell types in the absence of a carrier.

As described in the following Example, circular RNA containing RNA aptamer sequences within the circular RNA (where the RNA aptamer sequences target therapeutically-relevant proteins on a target cell) and a Gaussia Luciferase (GLuc) ORF enter cells in a carrier-independent manner.

Circular RNAs are designed to include either a C2 min aptamer sequence (5′-GGG GGA UCA AUC CAA GGG ACC CGG AAA CGC UCC CUU ACA CCC C-3′ (SEQ ID NO: 2)) known to bind competitively to the human transferrin receptor (TfR aptamer), a spacer region, and an EMCV IRES and GLuc ORF. In some circular RNA designs, the TfR aptamer is present in multiple copies. A circular RNA without an aptamer sequence is used as control.

The circular RNA is generated in vitro. Unmodified linear RNA is transcribed in vitro from a DNA template including all the motifs listed above, as well as a T7 RNA polymerase promoter to drive transcription. Transcribed RNA is purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA is circularized using a splint DNA (5′-GTCAACGGATTTTCCCAAGTCCGTAGCGTCTC-3′ (SEQ ID NO: 6)) and T4 RNA ligase. Circular RNA is urea-PAGE purified, eluted in a buffer, ethanol precipitated and resuspended in RNase storage solution (ThermoFisher Scientific, cat #AM7000).

Circular RNA containing aptamer sequences is added to NALM6 human peripheral blood cells (ATCC CRL-3273) grown under standard conditions (in RPMI-1640 (ATCC), with 10% FBS at 37° C. under 5% CO2). Circular RNA is transfected into the cells in the absence of a carrier. Multiple timepoints are studied. Cell media is collected for a luminescence assay to detect GLuc activity and replaced with fresh media after, 6 hour, 24 hour and 48 hours of incubation at 37° C. After 48 hours, the media is collected and RNA is extracted to analyze circular RNA level.

Efficiency of delivery of the circular RNA is measured in two different ways: (i) luminescence assay to detect GLuc activity, which correlates with GLuc protein expression from the circular RNA; and (ii) qRT-PCR for circular RNA delivery. To measure GLuc activity, luciferase activity is detected using a commercially available luminescence kit (e.g., Gaussia Luciferase Flash Assay Kit; Pierce 16159). Briefly, 10 uL of collected media for each sample is placed in the 96 well white plate. 50 μl of GLuc substrate in reaction buffer is added to each well containing media. Luminescence is read in a luminometer instrument (Promega). For RT-qPCR, a commercially available kit is used for reverse transcription (e.g., Power SYBR Green Cell-to-CT Kit; (Invitrogen, cat #4402953). qRT-PCR is performed with GLuc-specific primers (forward; CCTGAGATTCCTGGGTTCAAG (SEQ ID NO: 7) reverse; CTTCTTGAGCAGGTCAGAACA (SEQ ID NO: 8)) and a ready-to-use reaction master mix optimized for dye-based quantitative PCR (e.g., iTaq Universal SYBR Green Supermix; Bio-RAD, cat #1725120) and monitored by commercially available real-time PCR detection systems. Beta actin is used as reference (forward; GACGAGGCCCAGAGCAAGAGAGG (SEQ ID NO: 9), reverse; GGTGTTGAAGGTCTCAAACATG (SEQ ID NO: 10)).

### Example 4: Circular RNA Hybridized to Single-Stranded RNA Oligonucleotides Containing RNA Aptamer Sequences can Target Surface Proteins and Enable Uptake of the Circular RNA in the Absence of Carrier (Naked Delivery)

This Example describes targeting of circular RNA to therapeutically relevant proteins on a target cell via RNA aptamers sequences contained in a single-stranded RNA oligonucleotide that hybridizes to the circular RNA.

As described in the following Example, circular RNA comprising a GLuc ORF and hybridized to single-stranded RNA oligonucleotides containing RNA aptamer sequences (where the RNA aptamer sequences target therapeutically-relevant proteins on a target cell) enter cells in a carrier-independent manner.

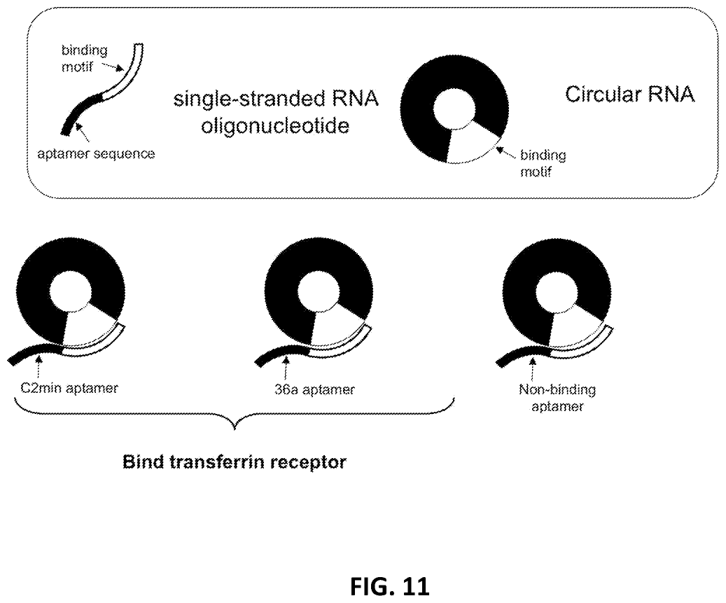

In this Example, the linear RNA oligonucleotide includes either a TfR aptamer sequence (5′-GGG GGA UCA AUC CAA GGG ACC CGG AAA CGC UCC CUU ACA CCC C-3′ (SEQ ID NO: 2)), known to bind competitively to the human transferrin receptor; or, a 36a (5′-GGG UGA AUG GUU CUA CGA UAA ACG UUA AUG ACC AGC UUA UGG CUG GCA GUU CCU AUA GCA CCC-3′ (SEQ ID NO: 3)) aptamer sequence known to bind non-competitively to the human transferrin receptor; or a negative control (5′-GGC GUA GUG AUU AUG AAU CGU GUG CUA AUA CAC GCC-3′ (SEQ ID NO: 11)). This linear RNA oligonucleotide also includes a binding motif for hybridization to the circular RNA. A schematic of these entities is shown in FIG. 11.

Six different oligonucleotides are designed that contain annealing regions that bind to different regions of circular RNA. Two of these oligonucleotides bind to the spacer region and four bind to the GLuc ORF. Circular RNAs include the complementary binding region for hybridization of the aptamer-containing single-stranded oligonucleotide as well as an EMCV IRES and GLuc ORF. A control complex is generated using the same circular RNA as described above that hybridizes to a single-stranded linear RNA oligonucleotide including an aptamer sequence that is predicted not to bind to human transferrin receptor.

The circular RNA is generated in vitro, as described in Example 3.

The single-stranded RNA oligonucleotide containing the TfR aptamer sequence and the binding motif is custom-synthesized by Integrated DNA Technologies (IDT). The aptamer sequence is synthesized as an unmodified nucleotide. The circular RNA binding sequence is designed to include 2′-O-methyl modified nucleotides in the annealing sequence.

The single-stranded RNA oligonucleotide is added at 2× molar excess over the circular RNA, incubated at 65° C. for 10 minutes and then gradually cooled to room temperature in buffer. Annealing is confirmed by agarose gel electrophoresis.

Circular RNA annealed with the aptamer-containing RNA oligonucleotide is added to NALM6 human peripheral blood cells (ATCC CRL-3273) grown under standard conditions (in RPMI-1640 (ATCC), with 10% FBS at 37° C. under 5% CO2). Circular RNA is transfected into the cells in the absence of a carrier. Multiple timepoints are studied. Cell media is collected for a luminescence assay to detect GLuc activity and replaced with fresh media after 6 hours, 24 hours and 48 hours of incubation at 37° C. Media is collected and RNA is extracted from the cells to analyze circular RNA level.

Efficiency of delivery of the circular RNA is measured as described in Example 3 using: (i) luminescence assay to detect GLuc activity, which correlates with GLuc protein expression from the circular RNA; and (ii) qRT-PCR for circular RNA delivery.

### Example 5: Circular RNA Hybridized to Single-Stranded RNA Oligonucleotides Containing RNA Aptamer Sequences can Target Surface Proteins and Enable Uptake of the Circular RNA in the Absence of Carrier (Naked Delivery)

This Example describes targeting of circular RNA to therapeutically relevant proteins on a target cell via RNA aptamers sequences contained in a single-stranded RNA oligonucleotide that hybridizes to the circular RNA.

As described in the following Example, circular RNA comprising a GLuc ORF and hybridized to single-stranded RNA oligonucleotides containing RNA aptamer sequences (where the RNA aptamer sequences target therapeutically-relevant proteins on a target cell) enter cells in a carrier-independent manner.

In this example, the linear RNA oligonucleotide includes a EpCAM aptamer sequence (5′-GCG ACU GGU UAC CCG GUC G-3′ (SEQ ID NO: 12)) known to bind competitively to the human EpCAM protein. Six different oligonucleotides are designed where each oligonucleotide binds to a different region of the circular RNA, two bind to spacer region and four bind to the GLuc ORF. Circular RNAs include the complementary binding region for hybridization of the aptamer-containing single-stranded oligonucleotide as well as an EMCV IRES and GLuc ORF. A control complex is generated using the same circular RNA as described above that hybridizes to a single-stranded linear RNA oligonucleotide including an aptamer sequence that is predicted not to bind to the human EpCAM protein.

The circular RNA is generated in vitro as described in Example 3.

The single-stranded RNA oligonucleotide containing the EpCAM aptamer sequence and binding motif is custom-synthesized by Integrated DNA Technologies (IDT).

The aptamer sequence is synthesized as unmodified purine and 2′fluoro pyrimidine. The circular RNA binding sequence is designed to include 2′-O-methyl modified nucleotides in the annealing sequence.

The single-stranded RNA oligonucleotide is added at 2× molar excess over the circular RNA, incubated at 65° C. for 10 minutes and then gradually cooled to room temperature in buffer. Annealing is confirmed by agarose gel electrophoresis.

Circular RNA annealed with the aptamer-containing RNA oligonucleotide is added to HCC1143 human breast cells (ATCC CRL-2321) grown under standard conditions (in RPMI-1640 (ATCC), with 10% FBS at 37° C. under 5% CO2); or (2) BT549 human mammary gland cells (ATCC HTB-122, grown under standard conditions (in RPMI-1640 media (ATCC), with 10% FBS at 37° C. under 5% CO2). HCC1143 shows high EpCAM surface expression but BT549 has little surface expression. Circular RNA is transfected into the cells in the absence of a carrier. Multiple timepoints are studied. Cell media is collected for a luminescence assay to detect GLuc activity and replaced with fresh media after 6 hours, 24 hours and 48 hours of incubation at 37° C. Media is collected and RNA is extracted from the cells to analyze circular RNA level.

Efficiency of delivery of the circular RNA is measured as described in Example 3 using: (i) luminescence assay to detect GLuc activity, which correlates with GLuc protein expression from the circular RNA; and (ii) qRT-PCR for RNA delivery.

### Example 6: Circular RNA Hybridized to Single-Stranded DNA Oligonucleotides Containing DNA Aptamer Sequences can Target Surface Proteins and Enable Uptake of the Circular RNA in the Absence of Carrier (Naked Delivery)

This Example describes targeting of circular RNA to therapeutically relevant proteins on a target cell via DNA aptamer sequences contained in a single-stranded DNA oligonucleotide that hybridizes to the circular RNA and causes the internalization of the circular RNA.

As described in the following Example, circular RNA comprising a GLuc ORF and hybridized to single-stranded DNA oligonucleotides containing DNA aptamer sequences (where the DNA aptamer sequences target therapeutically-relevant proteins on a target cell) enter cells in a carrier-independent manner.

In this Example, the linear DNA oligonucleotide includes a nucleolin aptamer sequence (5′-GGTGGTGGTGGTTGTGGTGGTGGTGG-3′ (SEQ ID NO: 13)) known to bind to cell surface nucleolin. This linear DNA oligonucleotide also includes a binding motif for hybridization to the circular RNA. Four different oligonucleotides are designed and each oligonucleotide binds to different region of circular RNA; two bind to spacer region and two bind to the GLuc ORF. Circular RNAs include the complementary binding region for hybridization of the aptamer-containing single-stranded oligonucleotide as well as an EMCV IRES and GLuc ORF.

The circular RNA is generated in vitro. Unmodified linear RNA is transcribed in vitro from a DNA template including all the motifs listed above, as well as a T7 RNA polymerase promoter to drive transcription. Transcribed RNA is purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA is circularized using a splint DNA (5′-GTCAACGGATTTTCCCAAGTCCGTAGCGTCTC-3′ (SEQ ID NO: 6)) and T4 RNA ligase. Circular RNA is urea-PAGE purified, eluted in a buffer, ethanol precipitated and resuspended in water.

The aptamer sequence is synthesized as DNA. The circular RNA binding sequence is designed to include 2′-O-methyl modified nucleotides in the annealing sequence.

The single-stranded RNA oligonucleotide is added at 2× molar excess over the circular RNA, incubated at 65° C. for 10 minutes and then gradually cooled to room temperature in buffer. Annealing is confirmed by agarose gel electrophoresis.

Circular RNA annealed with the aptamer-containing DNA oligonucleotide is added to (1) MCF-7 human breast cancer cells (ATCC HTB-22), grown under standard conditions (in Eagle's Minimum Essential Medium (EMEM; Sigma), with 10% FBS and 0.01 mg/mL human recombinant insulin at 37° C. under 5% CO2); or (2) MCF-10A human mammary epithelial cell (ATCC CRL-10317), grown under standard conditions (in MEBM™ Mammary Epithelial Cell Growth Basal Medium (Lonza/Clonetics Corporation) with 100 ng/mL cholera toxin under 5% CO2). MCF7 shows high Nucleolin surface expression but BT549 has little surface expression. Circular RNA is transfected into the cells in the absence of a carrier. Multiple timepoints are studied. Cell media is collected for a luminescence assay to detect GLuc activity and replaced with fresh media after 6 hours, 24 hours and 48 hours of incubation at 37° C. Media is collected and RNA is extracted from the cells to analyze circular RNA level.

Efficiency of delivery of the circular RNA is measured as described in Example 3 using: (i) luminescence assay to detect GLuc activity, which correlates with GLuc protein expression from the circular RNA; and (ii) qRT-PCR for circular RNA delivery.

### Example 7: Circular RNA Hybridized to Single-Stranded DNA Oligonucleotides Containing DNA Aptamer Sequences can Target Surface Proteins and Enable Uptake of the Circular RNA In Vivo in the Absence of Carrier (Naked Delivery)

This Example describes targeting circular RNA to therapeutically relevant proteins on a target cell in vivo via DNA aptamers sequences contained in a single-stranded DNA oligonucleotide that hybridizes to the circular RNA.

As described in the following Example, circular RNA comprising a GLuc ORF and hybridized to single-stranded DNA oligonucleotides containing DNA aptamer sequences (where the DNA aptamer sequences target therapeutically-relevant proteins on a target cell) enter cells in vivo in a carrier-independent manner.

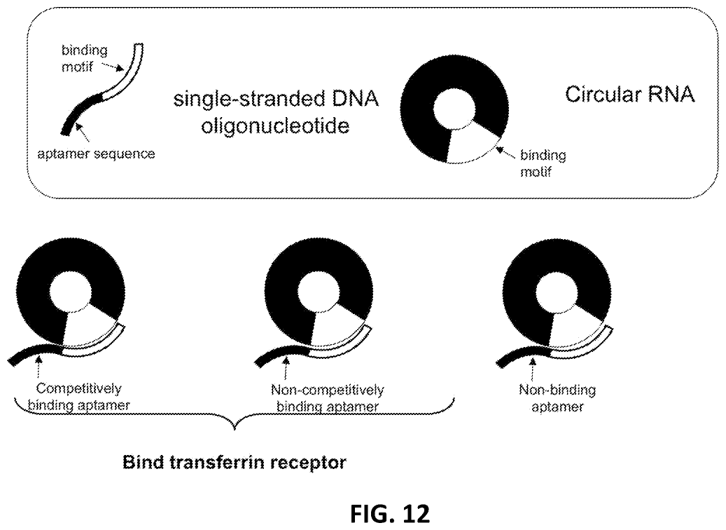

In this Example, a single stranded DNA oligonucleotide includes either an aptamer sequence known to bind competitively to the mouse transferrin receptor; or, an aptamer sequence known to bind non-competitively to the mouse transferrin receptor. This single stranded DNA oligonucleotide also includes a binding motif for hybridization to the circular RNA. Circular RNAs include the complementary binding region for hybridization of the aptamer-containing single-stranded oligonucleotide as well as an EMCV IRES and Gaussia Luciferase (GLuc) ORF. A control complex is generated using the same circular RNA as described above that hybridizes to a single-stranded DNA oligonucleotide including an aptamer sequence that is predicted not to bind to mouse transferrin receptor. A schematic of these entities is shown in FIG. 12.

The circular RNA is generated in vitro as described in Example 3.

In this example, the single-stranded DNA oligonucleotide is custom-synthesized by Integrated DNA Technologies (IDT) containing the aforementioned aptamer sequence and binding motif. The single stranded DNA oligonucleotide (1) is unmodified; or (2) contains 5′-fluoro modifications, as described in Kratschmer et al., (2017) Nucleic Acid Ther. 27(6):335-344; or (3) is modified to include modifications such as a 5′-hydroxyl moiety, or 2′-O-methyl modifications.

The single-stranded DNA oligonucleotide is added at 3× molar excess over the circular RNA, incubated at 60° C. for 10 minutes and then gradually cooled to room temperature in buffer. RNA buffer is exchanged to PBS using a Microbiospin column (Biorad). Annealing is confirmed by agarose gel electrophoresis.

PBS is then added to the desired final construct concentration of 25 pmole in a 100 uL final volume.

A single injection is received by the mice in the hind leg of 25 pmole of any of the hybridized single stranded DNA oligonucleotide and circular RNA constructs.

At 0, 1, 6 hours, 1, 2, 4, 7 days post-dosing, mice are sacrificed, and liver tissue is collected and stored in RNAlater solution (ThermoFisher Scientific, cat #AM7020) and frozen until processing. To isolate total RNA, tissue samples are homogenized in trizol, phenol-chloroform extracted, and the aqueous-phase is then column purified using an RNA cleanup kit (New England Biolabs, T2050). cDNA is synthesized from the total RNA using Superscipt IV (Thermo Scientific, cat #18091050). qRT-PCR is performed on cDNA templates using primers specific to the GLuc ORF. Primers against the housekeeping gene, mouse 28s rRNA, are used for normalization (F: GGTTGAGGGCCACCTTATTT (SEQ ID NO: 14), R: GAAGAAAGACCGGGAAGAGAAA (SEQ ID NO: 15)). The delta-delta Ct method is used to calculate relative gene expression and absolute numbers will be obtained using a standard curve.

It is expected that cells incubated with circular RNA hybridized to the competitively binding aptamer containing single-stranded DNA oligonucleotide or the non-competitively binding aptamer containing single-stranded RNA oligonucleotide show increased internalization into hepatocytes and liver tissue cells compared to the circular RNA hybridized to the non-binding aptamer containing single-stranded DNA oligonucleotide.

This Example describes that DNA aptamer sequences encoded within a single-stranded DNA oligonucleotide that hybridizes to the circular RNA can increase uptake into mammalian cells in vivo without a transfection agent via interaction with the specific surface receptor.

### Example 8: Circular RNA that Binds a Lipid Membrane

This Example describes circular RNA binding to a lipid membrane.

Circular RNA can be designed to specifically bind to lipid membranes. The following Example describes a circular RNA binding to a membrane. By mediating binding of cellular membranes, circular RNA is able to bring adjacent cells into close proximity of one another.

Circular RNA is designed to include at least one RNA motif (sequences described herein) that is designed to bind a membrane:

(SEQ ID NO: 16)
GUGAUGGCGCCUACGUCGAAGAAAGGAGUCUCAAGGGAAGGAGCGUAUAU
GGUCGAUGAAUCGGUCAUGUCGUCAGGGU;
(SEQ ID NO: 17)
GAGUCAUAGGACGCUCGCUCUUGCGACCAUGGGGCACGGGGAGCCCACUG
CAUGGAUCU AUCGUAU CAUAGUGCGGU;
(SEQ ID NO: 18)
GUAGCUUCCAUGAGACUUGAUCGGGGUCAUGGCUCUAGGCAUCGGAGAAG
CUGACUAACU UGGUCACGUCGUACCUGGU;
(SEQ ID NO: 19)
GGACGCGUACGAAGGGCUGAUAGGGCAGAGCUCCAACUAUGCGUCCAGCU
CGUGCAGUGGAUCGGGUCGUGCCUGGU;
and
(SEQ ID NO: 20)
CUUUGUCGGCCGAACUCGCUGUUUAACUGCCCGGCGAGAUCGCAGGGUGU
UGUGCUAUU CGCGUGCCGUGUG.

Unmodified linear RNA is synthesized by in vitro transcription using T7 RNA polymerase from a DNA segment comprising one or more of the RNA lipid binding motifs. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M), and the circular RNA is isolated following enrichment with RNase R treatment. RNA quality is assessed by agarose gel or through automated electrophoresis (Agilent).

One method to assess circular RNA binding to a lipid membrane is incubation of the circular RNAs with liposomes. Liposomes are fractionated using a Sephacryl S-1000 column. All unbound RNA is discarded. Bound circular RNA is assessed through qPCR, or northern blotting.

### Example 9: Circular RNA that Binds Carbohydrates

This Example describes circular RNA binding to carbohydrates.

Sialyl Lewis X is a tetrasaccharide glycoconjugate of membrane proteins. It acts as a ligand for selectin proteins during cell adhesion. As shown in the following Example, the circular RNA binds to Sialyl Lewis X to inhibit cell adhesion.

An engineered circular RNA is designed to include a Sialyl Lewis X binding sequence (e.g., 5′-CCGUAAUACGACUCACUAUAGGGGAGCUCGGUACCGAAUUCAAGGUACUCUGUGCUUGU CGAUGUGUAUUGAUGGCACUUUCGAGUCAACGAGUUGACAGAACAAGUAGUCAAGCUUU GCAGAGAGGAUCCUU-3′ (SEQ ID NO: 21)).

Unmodified linear RNA is synthesized by in vitro transcription using T7 RNA polymerase from a DNA segment comprising Sialyl Lewis X binding sequence. Transcribed RNA is purified with an RNA purification system (QIAGEN), treated with alkaline phosphatase (ThermoFisher Scientific, EF0652) following the manufacturer's instructions, and purified again with the RNA purification system. Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to Sialyl Lewis X is to measure Sialyl Lews X-mediated cell adhesion. E-selectin recognizes Sialyl Lews X, and the surface of promyelocytic leukemia cell line HL60 is rich in Sialyl Lews X, especially after TNF-α treatment. Recombinant soluble E-selectin (Calbiochem) is added to the microtiter plate (250 ng/well) in 0.05 M NaHCO₃ at pH 9.2 (10 μg/ml) and is incubated overnight at 4° C. Circular RNA (10 μg/mL) with or without the Sialyl Lewis X binding site is then incubated. TNF-α activated (10 ng/ml for 20 h) HL60 human promyelocytic leukemia cells are incubated for 30 min at room temperature on the plate, are washed, and the numbers of adhered cells are measured.

### Example 10: Circular RNA that Binds Virus

This Example describes circular RNA binding to virus.

The influenza virus has two membrane glycoprotein components including hemagglutinin (HA) and neuraminidase (NA). About 900 and 300 copies of HA and NA, respectively, are expressed on the surface of each viral particle. As shown in the following Example, an engineered circular RNA is designed to bind to hemagglutinin for viral binding.

Circular RNA is designed to include a Hemagglutinin binding site (e.g., 5′-GGGAGAAUUCCGACCAGAAGGGUUAGCAGUCGGCAUGCGGUACAGACAGACCUUUCCUC UCUCCUUCCUCUUCU-3′ (SEQ ID NO: 22)) to bind to the surface of the influenza virus.

Unmodified linear RNA is synthesized by in vitro transcription using T7 RNA polymerase from a DNA segment comprising hemagglutinin binding sequence. Transcribed RNA is purified with an RNA purification system (QIAGEN), treated with alkaline phosphatase (ThermoFisher Scientific, EF0652) following the manufacturer's instructions, and purified again with the RNA purification system. Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel electrophoresis or through automated electrophoresis (Agilent).

One method to assess circular RNA binding to hemagglutinin is inhibitory effects of RNA aptamers on HA-induced membrane fusion. When hemagglutinin is bound to circular RNA, membrane fusion occurs less frequently than that of unbound circular RNA.

HA-induced membrane fusion is examined by using fluorescently labelled virus and human red blood cell (RBC) ghost membranes. The viral membrane of A/Panama/2007/1999 (H3N2) is labelled with a fluorescent lipid probe, octadecyl rhodamine B (R18; Molecular Probes).

For the fusion-inhibition assay, the H3N2 virus (0.05-0.1 mg total protein/ml) mixed with a circular RNA (0.5 or 5 mM) is added to ghost membranes on coverslips mounted in a metal chamber. Upon viral fusion with ghost membranes, lipid intermixing between the viral and ghost membranes induces fluorescence dequenching of R18.

### Example 11: Circular RNA that Binds Aptamer

This Example describes circular RNA binding to an aptamer.

An engineered circular RNA is designed to include one or more novel binding sequences for RNA aptamers. RNA aptamers are targeted for circular RNA binding through complementarity. As shown in the following Example, the circular RNA binds complementary to the LIN28A binding aptamer for sequestration.

Circular RNA is designed to include the complementary sequence to the LIN28A binding aptamer sequence, 5′-GGGGUAGUGAUUUUACCCUGGAGAU-3′ (SEQ ID NO: 23).

Unmodified linear RNA is synthesized by in vitro transcription using T7 RNA polymerase from a DNA segment having the complementary LIN28A binding aptamer sequence. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

Circular RNA binding to the LIN28A binding aptamer is evaluated by an oligonucleotide pull-down-qPCR assay, in which modified oligonucleotides complementary to the circular RNA are used to pull-down the LIN28A binding aptamer, which is reverse-transcribed and qPCR amplified.

### Example 12: Circular RNA that Binds Cells

This Example describes circular RNA binding to target cell types.

In this Example, an engineered circular RNA is designed through one of the methods described previously. Circular RNA and linear RNA are designed to include a mango aptamer, a stabilizing stem, and a non-coding region: a transferrin aptamer (e.g., GGGGGAUCAAUCCAAGGGACCCGGAAACGCUCCCUUACACCCC (SEQ ID NO: 2)). This aptamer region binds the transferrin receptor allowing the RNA to bind to cells that express the receptor. Transferrin receptor is expressed on a variety of cell-types, including red blood cells and some cancer cells. As a negative control, RNA is designed to not include the aptamer region.

HeLa cells are cervical cancer cells that are known to express the transferrin receptor. HeLa cells are grown under standard conditions (in DMEM, with 10% FBS at 37° C. under 5% CO2). Cells are passaged regularly to maintain exponential growth. Circular RNA binding to TO-1 biotin is evaluated in vitro in HeLa cells, using fluorescent microscopy. When TO-1 biotin is bound to RNA it enhances its fluorescence more than 100-fold. Circular RNA with or without aptamers (50 nM) is added to the media of HeLa cultures, as well as a no-RNA control. A lipid-based transfection reagent (Thermo Fisher Scientific) is added to ensure RNA delivery. Cultures are treated with TO-1 biotin and fluorescence is analyzed after 3 and 6 hours.

### Example 13: Circular RNA for siRNA Delivery

This Example describes circular RNA delivering several siRNAs.

A non-naturally occurring circular RNA is engineered to include siRNA sequences that bind to the model target Transthyretin (TTR) mRNA. The following Example describes the circular RNA derived siRNAs binding to the target TTR mRNA to inhibit of transthyretin protein translation.

Circular RNA is designed to include sequences complementary to TTR mRNA (e.g. auggaauacu cuugguuactt), which bind to transthyretin mRNA resulting in the cleavage of this mRNA.

Unmodified linear RNA is synthesized by in vitro transcription using T7 RNA polymerase from a DNA segment having TTR complementary sequence. Transcribed RNA is purified with an RNA purification system (QIAGEN), treated with alkaline phosphatase (ThermoFisher Scientific, EF0652) following the manufacturer's instructions, and purified again with the RNA purification system.

To generate circular RNA, the two RNA ends, bearing a 5′-phosphate and 3′-OH are designed with additional flanking complementary sequences. These complementary sequences hybridize, resulting in a nicked circle. This nick is closed by T4 DNA ligase. Circular RNA quality is assessed by agarose or PAGE gel, or through automated electrophoresis (Agilent).

Circular RNA binding to TTR mRNA is evaluated by pull-down of circular RNA using a biotinylated oligo complementary to a specific sequence within the circle followed by RT-PCR. siRNA function is evaluated by measuring TTR target mRNA levels by RT-PCR in treated vs untreated cells. Expression of TTR protein is evaluated by western blotting.

### Example 14: Circular RNA with Modified Nucleotides was Generated and Selectively Bound Proteins

This Example demonstrates the generation of modified circular polyribonucleotide that supported protein binding. In addition, this Example demonstrates that circular RNA engineered with nucleotide modifications that selectively interacted with proteins involved in immune system monitoring had reduced immunogenicity as compared to unmodified RNA.

A non-naturally occurring circular RNA engineered to include complete or partial incorporation of modified nucleotides was produced. As shown in the following Example, full length modified linear RNA or a hybrid of modified and unmodified linear RNA was circularized and protein scaffolding was assessed through measurements of nLuc expression. In addition, selectively modified circular RNA had reduced interactions with proteins that activate immune related genes (q-PCR of MDA5, OAS and IFN-beta expression) in BJ cells, as compared to an unmodified circular RNA.

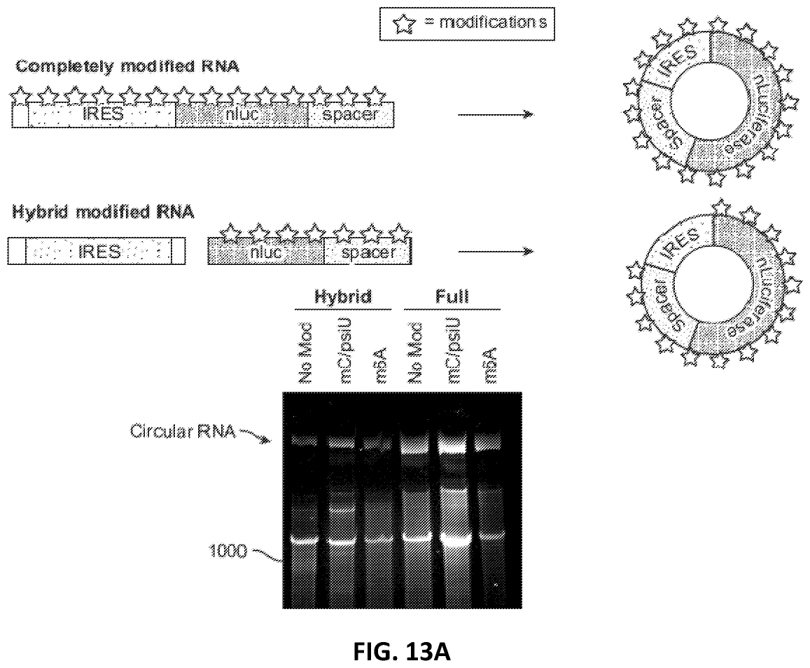

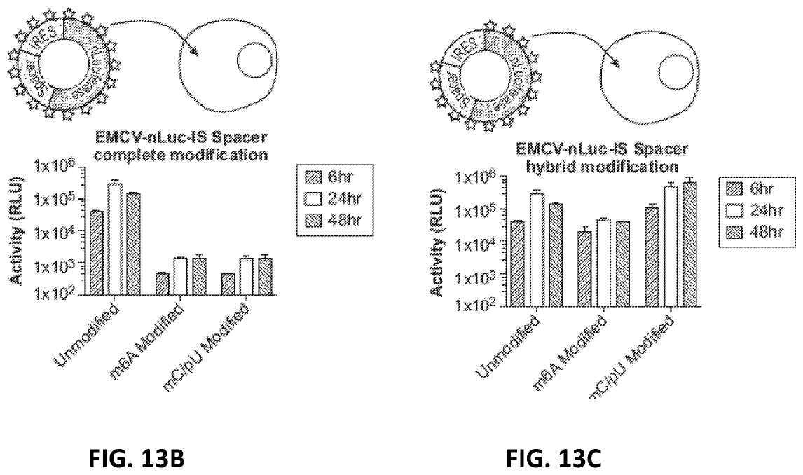

Circular RNA with a WT EMCV Nluc stop spacer was generated. For modification substitution, the modified nucleotides, pseudouridine and methylcytosine or m6A, were added in place of the standard unmodified nucleotides, uridine and cytosine or adenosine, respectively, during the in vitro transcription reaction. The WT EMCV IRES was synthesized separately from the nLuc ORF. The WT EMCV IRES was synthesized using either modified (completely modified) or unmodified nucleotides (hybrid modified). In contrast, the nLuc ORF sequence was synthesized using modified nucleotides, pseudouridine and methylcytosine or m6A, in place of the standard unmodified nucleotides, uridine and cytosine or adenosine, respectively, for the entire sequence during the in vitro transcription reaction. Following synthesis of the modified or unmodified IRES and the modified ORF, these two oligonucleotides were ligated together using T4 DNA ligase. As shown in FIG. 13A, completely modified (upper construct) or hybrid modified (lower construct) circular RNAs were generated.

To measure protein scaffolding efficiency, expression of nLuc from the completely modified or hybrid modified constructs was measured. After 0.1 pmol of linear and circular RNA was transfected into BJ fibroblasts for 6 h, nLuc expression was measured at 6 h, 24 h, 48 h and 72 h post-transfection.

As shown in FIG. 13B and FIG. 13C, completely modified circular RNA had greatly reduced protein binding capacity, as measured by protein translation output, as compared to unmodified circular RNA. In contrast, hybrid modification demonstrated as much as or increased binding to proteins, e.g., protein translation machinery.

To further measure protein scaffolding efficiency, completely modified circular RNA was transfected into cells and protein scaffolding to immune proteins was measured. The level of protein scaffolding to immune proteins that activate innate immune response genes was monitored in BJ cells transfected with unmodified circular RNA, or completely modified circular RNA with either pseudouridine and methylcytosine or m6A modifications. Total RNA was isolated from the cells using a phenol-based extraction reagent (Invitrogen) and subjected to reverse transcription to generate cDNA. qRT-PCR analysis for immune related genes was performed using a dye-based quantitative PCR mix (BioRad).

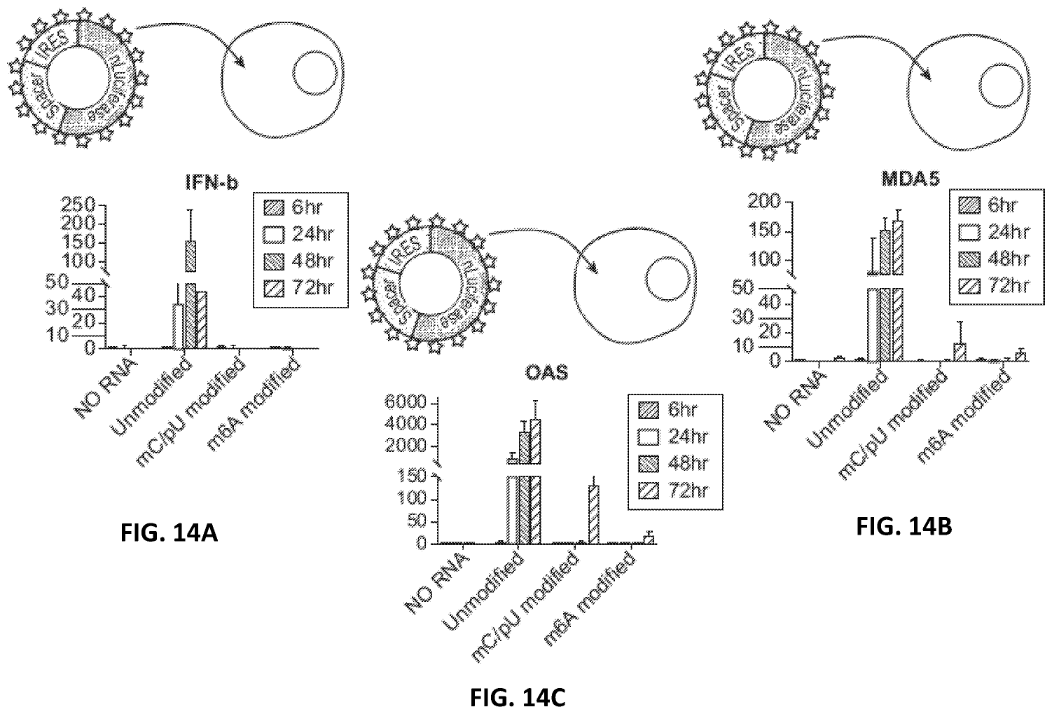

As shown in FIGS. 14A-C, qRT-PCR levels of immune related genes from BJ cells transfected with completely modified circular RNAs, both pseudouridine and methylcytosine or m6A completely modified circular RNAs, showed reduced levels of MDA5, OAS and IFN-beta expression as compared to unmodified circular RNA transfected cells, indicating reduced protein scaffolding between modified circular RNAs and immune proteins that activate immunogenic related genes. Thus, modification of circular RNA, as compared to unmodified circular RNA, had an impact on protein scaffolding. Selective modification allowed binding of protein translation machinery, while complete modification reduced binding to proteins that activate immunogenic related genes in transfected recipient cells.

### Example 15: Circular RNA with Modified Nucleotides Reduced Immunogenicity

This Example demonstrates the generation of modified circular polyribonucleotide that produced a protein product. In addition, this Example demonstrates circular RNA engineered with nucleotide modifications had reduced immunogenicity as compared to unmodified RNA.

A non-naturally occurring circular RNA engineered to include one or more desirable properties and with complete or partial incorporation of modified nucleotides was produced. As shown in the following Example, full length modified linear RNA or a hybrid of modified and unmodified linear RNA was circularized and expression of nLuc was assessed. In addition, modified circular RNA was shown to have reduced activation of immune related genes (q-PCR of MDA5, OAS and IFN-beta expression) in BJ cells, as compared to an unmodified circular RNA.

Circular RNA with a WT EMCV Nluc stop spacer was generated. For modification substitution, the modified nucleotides, pseudouridine and methylcytosine or m6A, were added in place of the standard unmodified nucleotides, uridine and cytosine or adenosine, respectively, during the in vitro transcription reaction. The WT EMCV IRES was synthesized separately from the nLuc ORF. The WT EMCV IRES was synthesized using either modified (completely modified) or unmodified nucleotides (hybrid modified). In contrast, the nLuc ORF sequence was synthesized using modified nucleotides, pseudouridine and methylcytosine or m6A, in place of the standard unmodified nucleotides, uridine and cytosine or adenosine, respectively, for the entire sequence during the in vitro transcription reaction. Following synthesis of the modified or unmodified IRES and the modified ORF, these two oligonucleotides were ligated together using T4 DNA ligase. As shown in FIG. 13A, hybrid modified circular RNAs were generated.

To measure expression efficiency, hybrid modified circular RNA was transfected into cells and expression of immune proteins was measured. Expression levels of innate immune response genes were monitored in BJ cells transfected with unmodified circular RNA, or hybrid modified circular RNAs with either pseudouridine and methylcytosine or m6A modifications. Total RNA was isolated from the cells using a phenol-based extraction reagent (Invitrogen) and subjected to reverse transcription to generate cDNA. qRT-PCR analysis for immune related genes was performed using a dye-based quantitative PCR mix (BioRad).

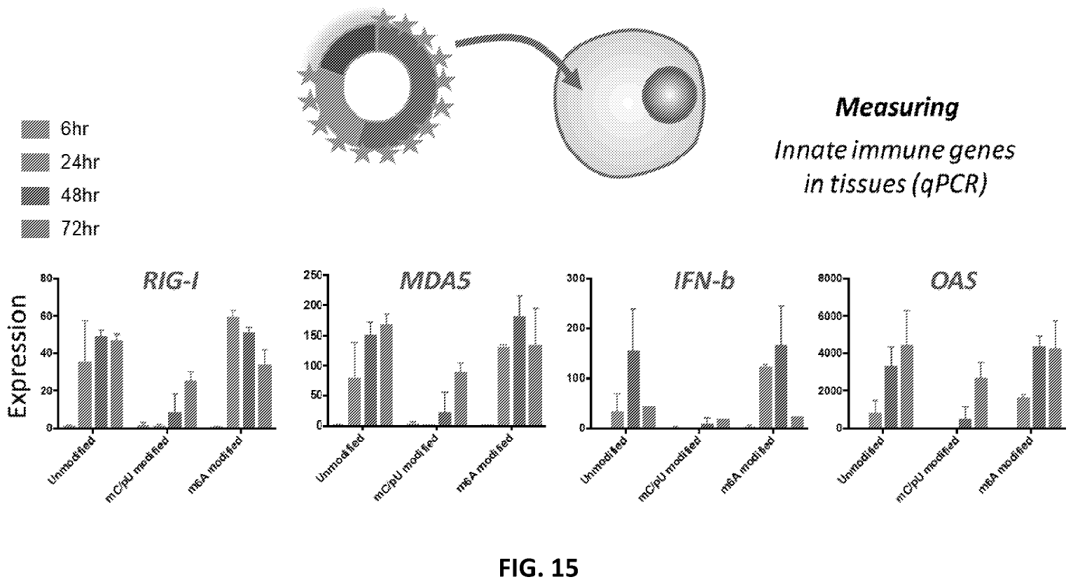

As shown in FIG. 15, qRT-PCR levels of immune related genes from BJ cells transfected with the hybrid modified circular RNAs, pseudouridine and methylcytosine hybrid modified circular RNAs showed reduced levels of RIG-I, MDA5, IFN-beta and OAS expression as compared to unmodified circular RNA transfected cells, indicating reduced immunogenicity of this hybrid modified circular RNA that activated the immunogenic related genes. Unlike the completely modified circular RNA shown in Example 24, m6A hybrid modified circular RNA showed similar levels of RIG-I, MDA5, IFN-beta and OAS expression as unmodified circular RNA transfected cells. Thus, modification of circular RNA, as compared to unmodified circular RNA, as well as the level of modification had an impact on activating immunogenic related genes.

### Example 16: Circular RNA Bound a Small Molecule

This Example demonstrates circular RNA binding a small molecule for sequestration/bio-activity.

Linear mango RNA aptamers fluoresce when bound by a small molecule, TO-1 biotin dye. As shown in the following Example, circular Mango RNA binds to the thiazol orange derivative, TO-1 biotin for sequestration/bio-activity.

Circular RNA was designed to include the mango RNA small molecule binding aptamer sites and a stabilizing stem: 5′-AATAGCCG GUCUACGGCC AUACCACCCU GAACGCGCCC GAUCUCGUCU GAUCUCGGAAGCUAAGCAGG GUCGGGCCUG GUUAGUACUU GGAUGGGAGA CCGCCUGGGAAUACCGGGUG CUGUAGGCGU CGACUUGCCA UGUGUAUGUG GGUACGAAGGAAGGAUUGGU AUGUGGUAUA UUCGUACCCA CAUACUCUGA UGAUCCUUCG GGAUCAUUCA UGGCAA CGGCTATT-3′ (SEQ ID NO: 1), as well as circularization sequences: 5′-AATAGCCG-3′ (SEQ ID NO: 24) and 5′-CGGCTATT-3′ (SEQ ID NO: 25).

Unmodified linear RNA was synthesized by in vitro transcription using T7 RNA polymerase from a DNA segment comprising the Mango RNA motif, stems and circularization sequences. Transcribed RNA was purified with an RNA cleanup kit (New England Biolabs, T2050), treated with RNA 5′-phosphohydrolase (RppH, New England Biolabs, M0356) following the manufacturer's instructions, and purified again with the RNA purification column. RppH treated RNA was circularized using a splint DNA complementary to the circularization sequences and T4 RNA ligase 2 (New England Biolabs, M0239). Circular RNA was Urea-PAGE purified, eluted in a buffer containing (0.5M Sodium Acetate, 0.1% SDS, 1 mM EDTA, ethanol precipitated and resuspended in RNase free water. RNA quality is assessed by Urea-PAGE or through automated electrophoresis (Agilent).

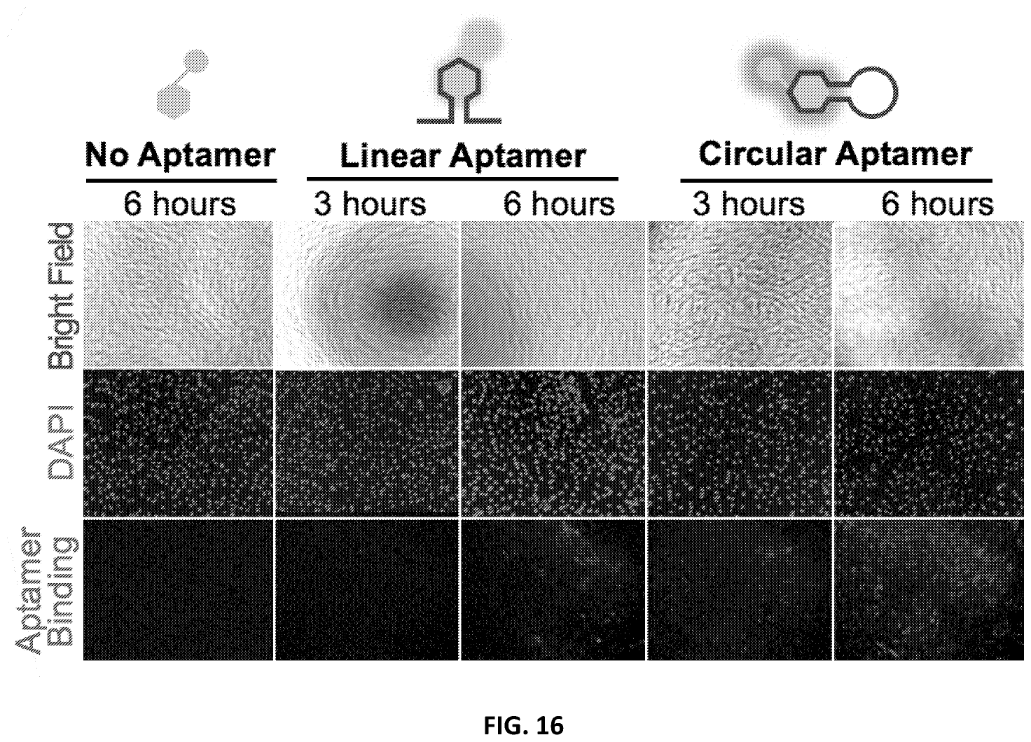

Circular RNA binding to TO-1 biotin was evaluated in vitro in BJ fibroblast cells, using fluorescent microscopy. When TO-1 biotin was bound to RNA it enhanced its fluorescence more than 100-fold. Linear or circular aptamers (50 nM) were added to the media of BJ fibroblast cultures, as well as a no-RNA control. A transfection reagent, lipofectamine, was added to ensure RNA delivery. Cultures were treated with TO-1 biotin and fluorescence was analyzed after 3 and 6 hours. As shown in FIG. 16, increased fluorescence/stability was detected from the circular aptamer, at both 3 and 6 hours.

More efficient delivery and more persistent fluorescence were observed with circular aptamers.

### Example 17: Circular RNA with a Small Molecule Bound a Protein

This Example demonstrates circular RNA linked to a small molecule to bound and recruited a protein of choice.

Thalidomide, a clinically approved drug (Thalomid), is known to associate a member of the cells' protein degradation machinery, the E3 ubiquitin ligase. By conjugating thalidomide to circular RNA (e.g., via click chemistry), thalidomide-conjugated circular RNA can recruit cells' degradation machinery to a second, disease-causing protein (e.g., also targeted by the circular RNA). As shown in the following Example, a small molecule was conjugated to a circular RNA to bind E3 ubiquitin ligase Cereblon.

Circular RNA was designed to include reactive uridine residues (e.g., 5-azido-C3-UTP) for conjugation of alkyne-functionalized small molecules, known to interact with an intracellular protein of interest.

Linear RNA was synthesized by in vitro transcription using T7 RNA polymerase (Lucigen). All UTP was substituted with 5-azido-C3-UTP (Jena Biosciences) in the in vitro transcription reaction to generate azide-functionalized RNA. Synthesized linear RNA was purified with an RNA clean up kit (New England Biolabs) and subjected to RNA 5′ Pyrophosphohydrolase (RppH, New England Biolabs) treatment to remove pyrophosphate. RppH-treated linear RNA was purified with an RNA clean up kit (New England Biolabs).

Circular RNA was generated by splint ligation. RppH-treated linear RNA (100 uM) and splint DNA (200 uM) was annealed by heating at 75° C. for 5 min and gradual cooling at room temperature for 20 min. Ligation reaction was performed with T4 RNA ligase 2 (0.2 U/ul, New England Biolabs) for 4 hours at 37° C. The ligated mixture was purified by ethanol precipitation. To isolate circular RNA, the ligated mixture was separated on 4% denaturing UREA-PAGE. RNA on the gel was stained with SYBR-green (Thermo Fisher) and visualized with transilluminator (Transilluminators). Corresponding RNA bands for circular RNA were excised and crushed by gel breaker tubes (Ist Engineering). For elution of circular RNA, crushed gels with circular RNA were incubated with elution buffer (0.5M Sodium Acetate, 1 mM EDTA, 0.1% SDS) at 37° C. for an hour and supernatant was carefully harvested. The remaining crushed gel elution was subjected to another round of elution, and repeated total three times. Elution buffer with circular RNA was filtrated through a 0.45 um cellulose acetate filter to remove gel debris and circular RNA was purified/concentrated by ethanol precipitation.

Alkyne-functionalized thalidomide (Jena Bioscience) was conjugated to azide-functionalized circular RNA via Copper-catalyzed Azide-Alkyne click chemistry reactions (CuAAC) with the click chemistry reaction kit based on manufacturer's instructions (Jena Bioscience). Thalidomide-conjugated circular RNA was purified with an RNA clean up kit (New England Biolab).

Binding properties of the thalidomide-conjugated circular RNA were analyzed using GST pull-down followed by qPCR for RNA detection. For GST pull-down assay, thalidomide-conjugated circular RNA (2 nM) was incubated with GST-E3 ubiquitin ligase Cereblon (50 nM), which interacts with thalidomide, for 2 hours at room temperature in the presence of 25 mM Tris-Cl (pH7.0), 100 mM NaCl, 1 mM EDTA, 0.5% NP-40, 5% Glycerol. Azide-functionalized circular RNA without thalidomide conjugation was used as a negative control.

The RNA-protein mixture was further incubated for an hour at room temperature with GSH-agarose beads to assess GST-GSH interactions. After washing three times with binding buffer, the RNA specifically bound to the GSH-beads was extracted with Trizol (Thermo Fisher). The extracted circular RNA was reverse transcribed and detected by quantitative RT-PCR with primers specific for circular RNA (forward: TACGCCTGCAACTGTGTTGT (SEQ ID NO: 26), reverse: TCGATGATCTTGTCGTCGTC (SEQ ID NO: 27)).

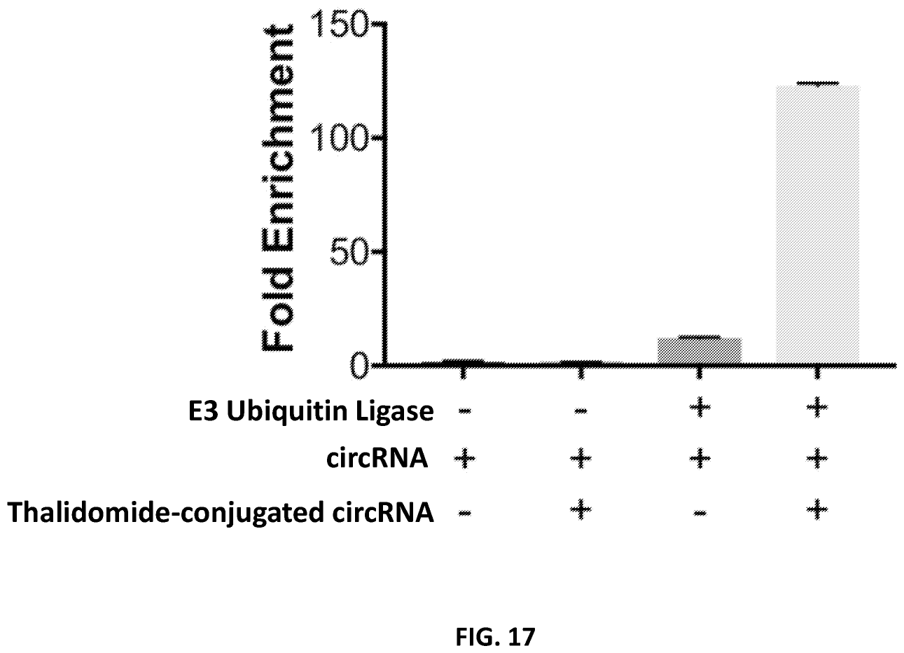

FIG. 17 demonstrates that circular RNA conjugated to the thalidomide small molecule was highly enriched in the GST pull-down assay, demonstrating that circular RNA with a small molecule, and bound to specific proteins through the small molecule.

### Example 18: Circular RNA Bound a Transcription Factor

This Example demonstrates circular RNA bound to protein for sequestration. NF-kB is a family of transcription factors that activate transcription and induce survival pathways. As shown in the following Example, the circular RNA bound to NF-kB for sequestration.

Circular RNA was designed to include the NF-kB RNA binding aptamer motifs: 5′-aaaaaaaaaaGATCTTGAAACTGTTTTAAGGTTGGCCGATCTTaaaaaa-3′ (SEQ ID NO: 28) to competitively bind NF-kB and inhibit its binding/downstream functions. Poly(A) stretches were added to the internal binding motif to (1) make the RNA oligo amenable to ligation and to maintain the secondary structure of the aptamer. Correct folding was checked using RNAfold WebServer. As a control, a scrambled RNA sequence was used (aaaaaaaTTCTCCGAACGTGTCACGTTTCAAGAGAACGTGACACGTTCGGAGAAaaaaaa (SEQ ID NO: 29)). This scrambled RNA sequence folds into a 3D structure similar to the aptamer, but does not target any proteins, as described in Mi et al., Mol Ther. 2008 January; 16(1):66-73.

RNA with the NF-kB binding aptamer motif was synthesized by a commercial vendor (IDT) with a 5′ monophosphate group and a 3′ hydroxyl group. RNA ligase 1 (New England Biolabs, M0204S) was used to ligate the RNA oligo. RNase R was used to remove residual linear RNA from the samples, according to manufacturer's instructions (Lucigen, RNR07250). Additionally, circular mRNA was purified by extracting the circular RNA from a 15% Urea PAGE gel. Circular RNA was eluted from the gel in a buffer containing: 0.5M Sodium Acetate, 0.1% SDS, 1 mM EDTA. Residual gel debris or salts from the gel extraction were removed by running the elution through a spin column (New England Biolabs, T2030S). RNA was eluted into RNA storage buffer (1 mM sodium citrate, Thermo Fisher, AM7000) and RNA integrity was assessed by Urea-PAGE or through automated gel capillary electrophoresis (Agilent).

Electrophoretic mobility shift assay (EMSA) was performed to assess circular RNA binding affinity to NF-kB. One pmole of linear or circular RNA was incubated with recombinant NF-kB p50 subunit (Caymen Chemical, 10009818) at varying concentrations over the RNA concentration (i.e., 0, 0.1, 1, 10 pmoles of protein) for 20 minutes at room temperature in a buffered reaction (20 mM Tris-HCl, pH 8.0, 50 mM NaCl, 1 mM MgCl2). Samples were run a 6% TBE Urea gel for 25 minutes at 200V. Gels were stained with SybrGold (Thermo Scientific, S11494) and imaged with a blue E-gel imaging system (Thermo Scientific, 4466612).

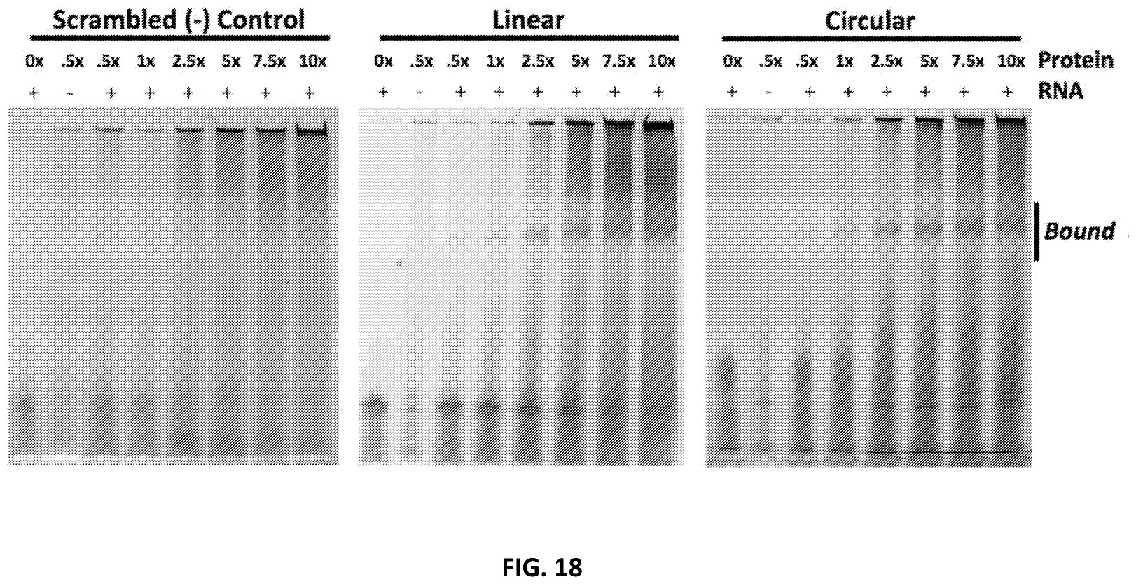

As demonstrated in FIG. 18, RNA with scrambled binding aptamer sequences did not show binding affinity to the p50 subunit of NF-kB. Both linear and circular versions of the NF-kB binding aptamer sequence bound to the p50 subunit with similar affinities.

Circular RNA binding to NF-kB was evaluated in vitro by EMSA for NF-kB. NF-kB selectively bound circular RNAs containing the NF-kB RNA binding aptamer motif. This result demonstrated that biomolecules of interests were selectively bound by sequences in circular RNA.

### Example 19: Circular RNA Sequestered Target Protein and Inhibited Function

This Example demonstrates circular RNA binds to protein in cells and this sequestration leads to inhibition of function. As shown in the following Example, the circular RNA binds to NF-kB for sequestration leading to inhibition of survival activated by NF-kB in cells.

Circular, linear, and linear scrambled RNA were designed and synthesized as previously described.

NF-kB function in non-small cell lung cancer (NSCLC) cell line, A549s, after delivery of a circular RNA with a NF-kB binding aptamer sequence was determined by measuring cell viability by MTT Assay (Thermo Scientific, V13154). In short, A549 cells were transfected with 1 pmole of linear, linear scrambled, or circular RNA after complexation with lipid transfection reagent (Thermo Scientific, LMRNA003). Viability was measured by MTT assay performed according to the manufacturer's instructions

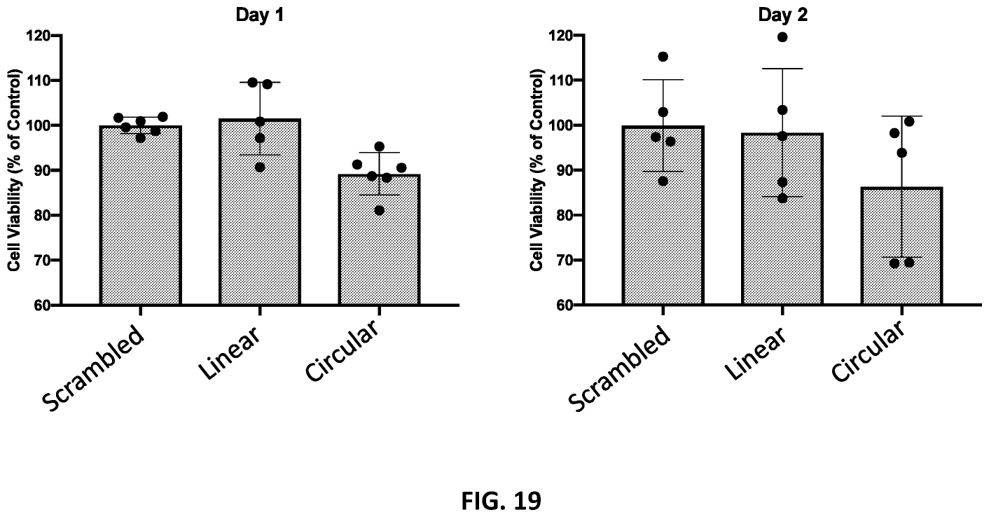

As demonstrated in FIG. 19, cells treated with linear RNA demonstrated no change in viability at day 1 and a slight decrease in viability at day 2 (101% viability on Day 1, and 97% on Day 2). In contrast, cells treated with the circular RNA demonstrated a measurable decrease in viability at day 1 and greater increase by day 2 (89% on Day 1 and 86% on Day 2).

Overall, the results demonstrated that circular RNA bound NF-kB in cells and inhibited NF-kB activation of survival pathways.

### Example 20: Circular RNA Bound and Sequestered Protein to Affect Chemotherapeutic Sensitization

This Example demonstrates circular RNA binds to a target protein in cells leading to the inhibition of the target protein's signaling pathways. As shown in the following Example, the circular RNA sequestered NF-kB in chemoresistant cells and inhibited NF-kB's signaling thereby re-sensitizing the cells to the chemotherapeutic.

Linear, linear scrambled, and circular RNA were designed and synthesized as previously described.

The effect of NF-kB sequestration in chemoresistant non-small cell lung cancer (NSCLC) cell line, A549s, was determined after delivery of a circular RNA targeting NF-kB and exposure to the chemotherapeutic agent. Cell viability was determined by MTT Assay (Thermo Scientific, V13154). In short, A549 cells were transfected with 1 pmole of a scrambled linear control, linear, or circular RNA after complexation with lipid transfection reagent (Thermo Scientific, LMRNA003). 24 hours post-transfection cells were treated with 5 uM doxorubicin for an additional 18 hours. Viability was measured by MTT assay performed according to the manufacturer's instructions. Doxorubicin treatment was repeated at 48- and 72-hours post transfection.

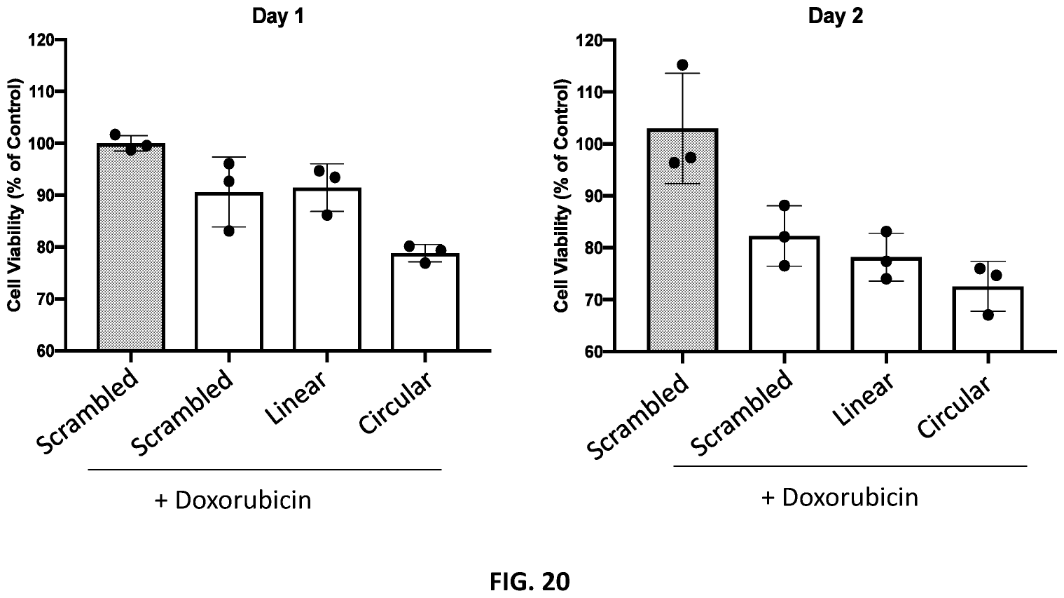

As demonstrated in FIG. 20, doxorubicin treatment with scrambled linear RNA (control) did not affect cell viability in the dox-resistant A549 lung cancer cell line at day 1. Co-treatment of doxorubicin with linear RNA decreased cell viability at day 2 (78% survival). In contrast, co-treatment with the circular aptamer resulted in more cell death at both days 1 and 2 (79% survival at day 1 and 73% survival at day 2).

Overall, the results demonstrated that circular RNA bound NF-kB in cells and inhibited NF-kB survival signaling, thereby increasing sensitivity of the cells to the chemotherapeutic, doxorubicin.

### Example 21: Circular RNA Tagged the Target Protein for Degradation

This Example demonstrates circular RNA linked to small molecules recruited two different proteins of choice and thereby tagged the target protein for degradation.

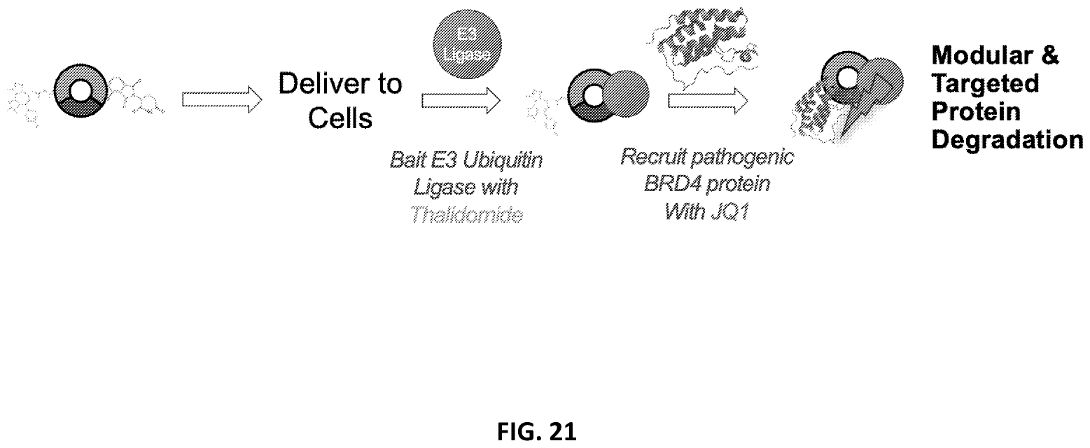

Thalidomide, a clinically approved drug (Revlimid), is known to associate with a member of the cells' protein degradation machinery, the E3 ubiquitin ligase. By conjugating thalidomide to circular RNA (e.g., via click chemistry), thalidomide-conjugated circular RNA can recruit cells' degradation machinery to a second, disease-causing protein (e.g., also targeted by the circular RNA). FIG. 21 is a schematic showing an exemplary circular RNA that is delivered into cells and tags a target BRD4 protein in the cells for degradation by ubiquitin system. As shown in the following Example, two small molecules (thalidomide and JQ1) were conjugated to a circular RNA to bind (1) E3 ubiquitin ligase Cereblon for ubiquitination and subsequent degradation of a neighboring protein; and (2) BET family proteins through JQ1 that is small molecule inhibitor that binds BET family proteins.

Circular RNA was designed to include multiple (49 residues) reactive uridine residues (e.g., 5-azido-C3-UTP) for conjugation of alkyne-functionalized small molecules, known to interact with an intracellular protein of interest.

Linear RNA was synthesized by in vitro transcription using T7 RNA polymerase (Lucigen). All UTP was substituted with 5-azido-C3-UTP (Jena Biosciences) in the in vitro transcription reaction to generate azide-functionalized RNA. Synthesized linear RNA was purified with an RNA clean up kit (New England Biolabs) and subjected to RNA 5′ Pyrophosphohydrolase (RppH, New England Biolabs) treatment to remove pyrophosphate. RppH-treated linear RNA was purified with an RNA clean up kit (New England Biolabs).

Circular RNA was generated by splint ligation. RppH-treated linear RNA (100 uM) and splint DNA (200 uM) was annealed by heating at 75° C. for 5 min and gradual cooling at room temperature for 20 min. Ligation reaction was performed with T4 RNA ligase 2 (0.2 U/ul, New England Biolabs) for 4 hours at 37° C. The ligated mixture was purified by ethanol precipitation. To isolate circular RNA, the ligated mixture was separated on 4% denaturing UREA-PAGE. RNA on the gel was stained with SYBR-green (Thermo Fisher) and visualized with transilluminator (Transilluminators). Corresponding RNA bands for circular RNA were excised and crushed by gel breaker tubes (Ist Engineering). For elution of circular RNA, crushed gels with circular RNA were incubated with elution buffer (0.5M Sodium Acetate, 1 mM EDTA, 0.1% SDS) at 37° C. for an hour and supernatant was carefully harvested. The remaining crushed gel was subjected to another round of elution, and repeated a total of three times. Elution buffer with circular RNA was filtrated through a 0.45p m cellulose acetate filter to remove gel debris and circular RNA was purified/concentrated by ethanol precipitation.

Alkyne-functionalized thalidomide and/or JQ1 (thienotriazolodiazepine, Jena Bioscience) was conjugated to azide-functionalized circular RNA via Copper-catalyzed Azide-Alkyne click chemistry reactions (CuAAC) with the click chemistry reaction kit based on manufacturer's instructions (Jena Bioscience). For comparison, three different kinds of small molecules were conjugated to circular RNA; RNA with both JQ1 and thalidomide, thalidomide only, or JQ1 only. Small molecule-conjugated circular RNA was purified with an RNA clean up kit (New England Biolab).

These different RNAs were then transfected into HEK293T cells to monitor degradation of target protein using by lipid transfection reagent (Invitrogen) according to the manufacturer's instruction. 1 pmole of each RNA was used to transfect HEK293T cells and the cells were plated into 12 well plates (2 nM final). In the case of circular RNA conjugated with both JQ1 and thalidomide, 3 pmole of RNA was transfected into HEK293T cells to test the effect of different concentrations of circular RNA on BRD4 degradation (6 nM final). As a positive control, PROTAC dBET1 (Tocris Biosciences) that has both JQ1 and thalidomide, and is known to degrade BRD4 protein in cells through CRBN recruitment, was used (2 uM, 10 uM concentration). For a negative control, carrier only and circular RNA without conjugation were used. After 24 hours transfection, cells were harvested by adding RIPA buffer directly onto the plate.

Small molecule-conjugated circular RNA binding to E3 ubiquitin ligase CRBN and BET family proteins degrading ability was analyzed using western blot. Briefly, 12 ug of protein was resolved on 4%-12% gradient Bis-Tris gel (Thermo Fisher Scientific) and transferred to nitrocellulose membrane using a blot transfer system (Thermo Fisher Scientific). Rabbit anti-BRD4 antibody (Abcam) was used to detect BRD4 protein and rabbit anti-alpha tubulin antibody (Abcam) was used to detect alpha tubulin as a loading control. The chemiluminescence signal from protein bands of BRD4 and alpha tubulin were monitored by an Fc imaging system (LI-COR).

BRD4 protein levels as well as alpha tubulin as a loading control were also measured using densitometry using ImageJ.

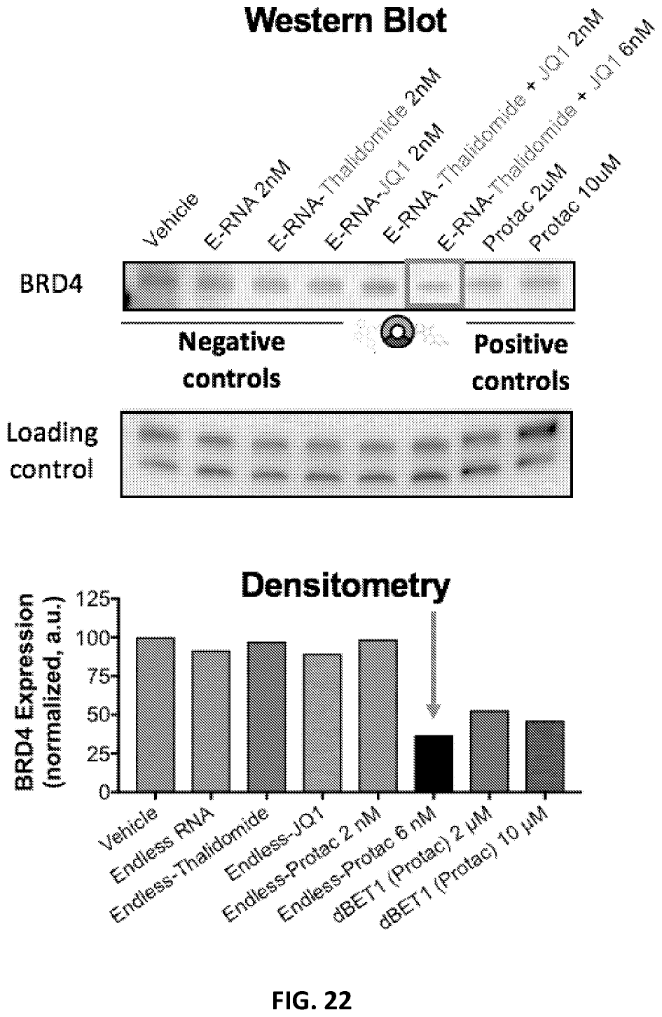

As shown in FIG. 22, circular RNA containing the thalidomide and JQ1 small molecules was able to degrade BRD4, as demonstrated by the normalized levels of BRD4. This result demonstrated that circular RNA with a small molecule bound to two specific proteins using the small molecule conjugate to degrade the target protein.

### Example 22: Circular RNA Bound a Small Molecule Longer than its Linear Counterpart

This Example demonstrates circular RNA binding a small molecule for sequestration/bio-activity. As shown in the following Example, the circular RNA is more stable than its linear counterpart.

Linear mango RNA aptamers fluoresce when bound by a small molecule, TO-1 biotin dye. As shown in the following Example, circular Mango RNA bound to the thiazol orange derivative, TO-1 biotin for sequestration/bio-activity.

Circular RNA was designed to include the mango RNA small molecule binding sites and a stabilizing stem: 5′-AATAGCCG GUCUACGGCC AUACCACCCU GAACGCGCCC GAUCUCGUCU GAUCUCGGAAGCUAAGCAGG GUCGGGCCUG GUUAGUACUU GGAUGGGAGA CCGCCUGGGAAUACCGGGUG CUGUAGGCGU CGACUUGCCA UGUGUAUGUG GGUACGAAGGAAGGAUUGGU AUGUGGUAUA UUCGUACCCA CAUACUCUGA UGAUCCUUCG GGAUCAUUCA UGGCAA CGGCTATT-3′ (SEQ ID NO: 1), as well as circularization sequences: 5′-AATAGCCG-3′(SEQ ID NO: 24) and 5′-CGGCTATT-3′ (SEQ ID NO: 25).

Unmodified linear RNA was synthesized by in vitro transcription using T7 RNA polymerase from a DNA segment comprising the Mango RNA motif, stems and circularization sequences. Transcribed RNA was purified with an RNA cleanup kit (New England Biolabs, T2050), treated with RNA 5′-phosphohydrolase (RppH, New England Biolabs, M0356) following the manufacturer's instructions, and purified again with the RNA purification column. RppH treated RNA was circularized using a splint DNA complementary to the circularization sequences and T4 RNA ligase 2 (New England Biolabs, M0239). Circular RNA was Urea-PAGE purified, eluted in a buffer containing (0.5M Sodium Acetate, 0.1% SDS, 1 mM EDTA, ethanol precipitated and resuspended in RNase free water. RNA quality was assessed by Urea-PAGE or through automated electrophoresis (Agilent).

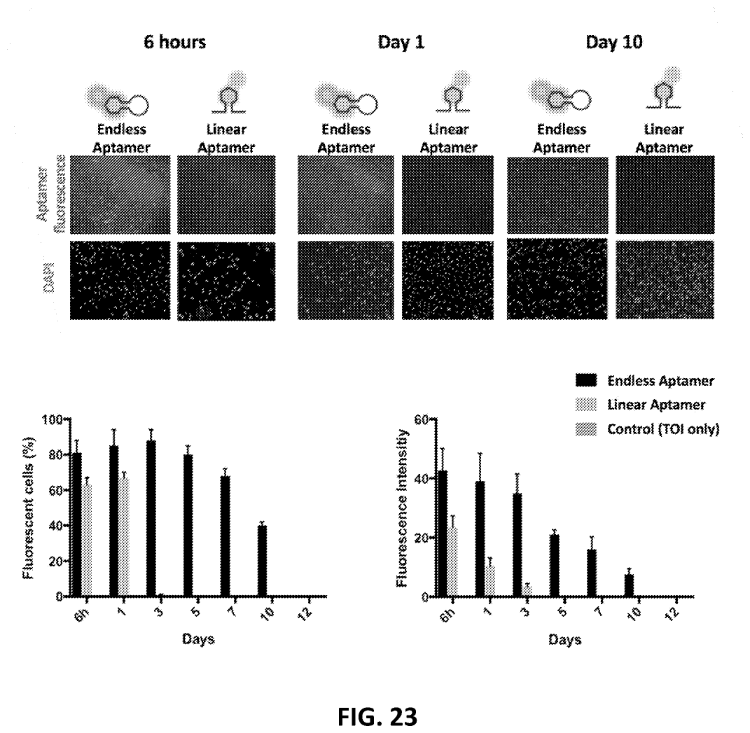

Circular RNA binding to TO-1 biotin was evaluated in vitro in HeLa cells, using fluorescent microscopy. When TO-1 biotin was bound to RNA it enhanced its fluorescence more than 100-fold. Linear or circular aptamers (50 nM) were added to the media of BJ fibroblast cultures, as well as a no-RNA control. A transfection reagent, lipofectamine, was added to ensure RNA delivery. Cultures were treated with TO-1 biotin and fluorescence was analyzed at 6 h and days 1-12. As shown in FIG. 23, increased fluorescence/stability was detected from the circular aptamer, with fluorescence detected at least for 10 days in culture.

### Example 23: Circular RNA Bound Protein and RNA

This Example demonstrates circular RNA binding to protein and RNA for sequestration.

Human antigen receptor (HuR) can be a pathogenic protein, e.g., it is known to bind and stabilize cancer related mRNA transcripts, such as mRNAs for proto-oncogenes, cytokines, growth factors, and invasion factors. HuR has a central tumorigenic activity by enabling multiple cancer phenotypes. Sequestration of HuR with circular RNA may attenuate tumorigenic growth in multiple cancers.

RNA plays a central role in cell metabolism and RNA molecules undergo multiple post-transcriptional processes, such as splicing, editing, modification, translation, and degradation.

As shown in the following Example, circular RNA binds to HuR and RNA for sequestration.

Circular RNA was designed to include the HuR RNA binding motif: 5′-UCAUAAUCAA UUUAUUAUUUUCUUUUAUUUUAUUCACAUAAUUUUGUUUUU-3′ (SEQ ID NO: 30) to competitively bind HuR and inhibit its binding/downstream functions and the RNA binding motif: 5′-CGA GAC GCT ACG GAC TTA AAA TCC GTT GAC-3′ (SEQ ID NO: 31).

Unmodified linear RNA was synthesized by in vitro transcription using T7 RNA polymerase from a DNA segment comprising the HuR RNA motif and protein binding sequence.

Circular RNA was designed to include the HuR RNA binding aptamer motif: 5′-UCAUAAUCAA UUUAUUAUUUUCUUUUAUUUUAUUCACAUAAUUUUGUUUUU-3′(SEQ ID NO: 30) to competitively bind HuR and inhibit its binding/downstream functions and the RNA binding aptamer motif: 5′-CGA GAC GCT ACG GAC TTA AAA TCC GTT GAC-3′ (SEQ ID NO: 31).

Unmodified linear RNA was synthesized by in vitro transcription using T7 RNA polymerase from a DNA segment comprising the HuR RNA motif and protein binding sequence.

Transcribed RNA was purified with an RNA cleanup kit (New England Biolabs, T2050), treated with RNA 5′-phosphohydrolase (RppH, New England Biolabs, M0356) following the manufacturer's instructions, and purified again with the RNA purification column. RppH treated RNA was circularized using a splint DNA complementary to the circularization sequences and T4 RNA ligase 2 (New England Biolabs, M0239). Circular RNA was Urea-PAGE purified, eluted in a buffer containing (0.5M Sodium Acetate, 0.1% SDS, 1 mM EDTA, ethanol precipitated and resuspended in RNase free water. RNA quality was assessed by Urea-PAGE or through automated electrophoresis (Agilent).

Circular RNA binding to HuR and RNA was evaluated in vitro by a combination of HuR immunoprecipitation (IP) and Biotin RNA pull-down assay, followed by qPCR. HuR protein-coupled to Protein G-anti HuR antibody was incubated with circular RNA, washed and eluted at low pH. Bound material was incubated with biotinylated RNA, washed and pulled down with streptavidin dynabeads.

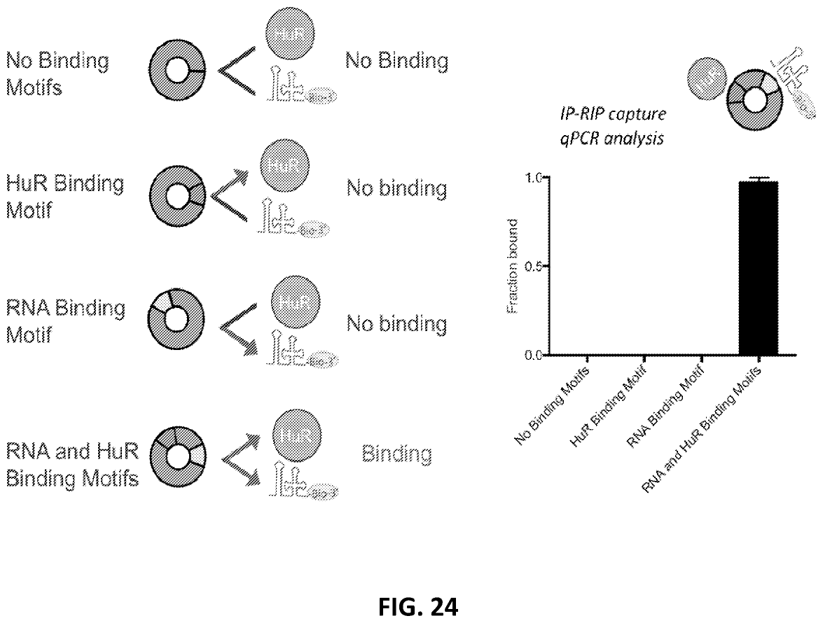

HuR bound circular RNAs with the HuR RNA binding aptamer motif and the streptavidin pull-down yielded RNAs with the RNA binding aptamer motifs as shown in FIG. 24. Thus binding was observed when the two, HuR and RNA, binding motifs were present. This result demonstrated that biomolecules of interests were selectively bound.

### Example 24: Circular RNA Bound Protein and DNA

This Example demonstrates circular RNA binding to protein and DNA for sequestration.

DNA binding by proteins and RNAs plays a pivotal role in different cellular processes, i.e., transcription.

Human antigen receptor (HuR) plays a central role in mRNA fate and plays a key role in post-transcriptional regulation of mRNA targets with central cellular functions, making it an important protein in pathogenesis. It is known to bind and stabilize cancer related mRNA transcripts, thus, HuR has a central tumorigenic activity by enabling multiple cancer phenotypes.

Targeting and competing these contacts with circular RNA could be used to modulate these interactions and control outcomes in disease and non-disease processes.

Circular RNA was designed to include the DNA binding aptamer motif: 5′-CGA GAC GCT ACG GAC TTA AAA TCC GTT GAC-3′ RNA (SEQ ID NO: 31).

Unmodified linear RNA was synthesized by in vitro transcription using T7 RNA polymerase from a DNA segment. Transcribed RNA was purified with an RNA cleanup kit (New England Biolabs, T2050), treated with RNA 5′-phosphohydrolase (RppH, New England Biolabs, M0356) following the manufacturer's instructions, and purified again with the RNA purification column. RppH treated RNA was circularized using a splint DNA complementary to the circularization sequences and T4 RNA ligase 2 (New England Biolabs, M0239). Circular RNA was Urea-PAGE purified, eluted in a buffer containing (0.5M Sodium Acetate, 0.1% SDS, 1 mM EDTA, ethanol precipitated and resuspended in RNase free water. RNA quality was assessed by Urea-PAGE.

Circular RNA binding to DNA and HuR was evaluated in vitro by a combination of HuR immunoprecipitation (IP) and biotinylated DNA pull-down assay, followed by RT-qPCR. Circular RNA lacking the DNA binding motif or HuR motif was used as a specificity control. The biotinylated DNA bound circular RNAs with the DNA binding aptamer motif.

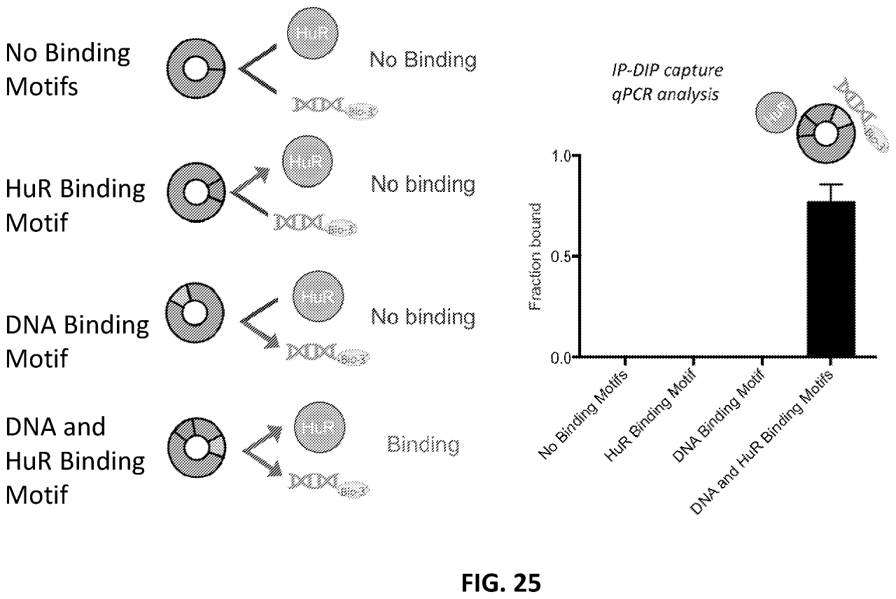

HuR protein-coupled to Protein G-anti-HuR beads was incubated with the circular RNA, washed and eluted at low pH. Bound material was incubated with biotinylated DNA, washed and pulled down with streptavidin Dynabeads. HuR bound circular RNAs with the HuR DNA binding aptamer motif and the streptavidin pull-down yielded RNAs with the DNA binding aptamer motifs as shown in FIG. 25. Thus, binding was observed when the two, HuR and DNA, binding aptamer motifs were present. This result demonstrated protein and DNA molecules of interests were selectively bound to the same circular construct.

### Example 25: Circular RNA Translated a Protein, and Bound to a Different Protein that Affected its Translation

This Example demonstrates circular RNA encoding a protein and binding a different protein that has an effect in circular RNA translation.

Human antigen receptor (HuR) plays a central role in mRNA fate and plays a key role in post-transcriptional regulation of mRNA targets with central cellular functions. Thus, using HuR to control RNA expression may provide control over translated protein dosage.

As shown in the following Example, a non-naturally occurring circular RNA was engineered to encode Gaussia Luciferase (GLuc), a biologically active secreted protein and to bind HuR to regulate GLuc translation. This circular RNA included an IRES, an ORF encoding Gaussia Luciferase, two spacer elements flanking the IRES-ORF and 1×, 2× or 3×HuR binding aptamer motifs: 5′-UCA UAA UCA AUU UAU UAU UUU CUU UUA UUU UAU UCA CAU AAU UUU GUU UUU-3′ (SEQ ID NO: 32), 5′-AUU UUG UUU UUA ACA UUUC-3′ (SEQ ID NO: 33), 5′-UCA UAA UCA AUU UAU UAU UUU CUU UUA UUU UAU UCA CAU AAU UUU GUU UUU AUU UUG UUU UUA ACA UUU C-3′ (SEQ ID NO: 34) to bind HuR.

Unmodified linear RNA was synthesized by in vitro transcription using T7 RNA polymerase from a DNA segment comprising the HuR RNA motif and protein binding sequence.

Transcribed RNA was purified with an RNA cleanup kit (New England Biolabs, T2050), treated with RNA 5′-phosphohydrolase (RppH, New England Biolabs, M0356) following the manufacturer's instructions, and purified again with the RNA purification column. RppH treated RNA was circularized using a splint DNA complementary to the circularization sequences and T4 RNA ligase 2 (New England Biolabs, M0239). Circular RNA was Urea-PAGE purified, eluted in a buffer containing (0.5M Sodium Acetate, 0.1% SDS, 1 mM EDTA, ethanol precipitated and resuspended in RNase free water. RNA quality was assessed by Urea-PAGE or through automated electrophoresis (Agilent).

Circular RNA binding to HuR was determined by in vitro RNA pull-down assay as described previously.

To evaluate the effect of HuR binding and its effect on circular RNA protein expression in cells, 5×10³ HeLa cells were successfully reverse transfected with a lipid-based transfection reagent (Invitrogen) and 2 nM of circular RNA. Gaussia Luciferase activity was monitored daily for up to 96 h in cell culture supernatants, as a measure of expression, using a Gaussia Luciferase assay kit and following manufacturer's instructions.

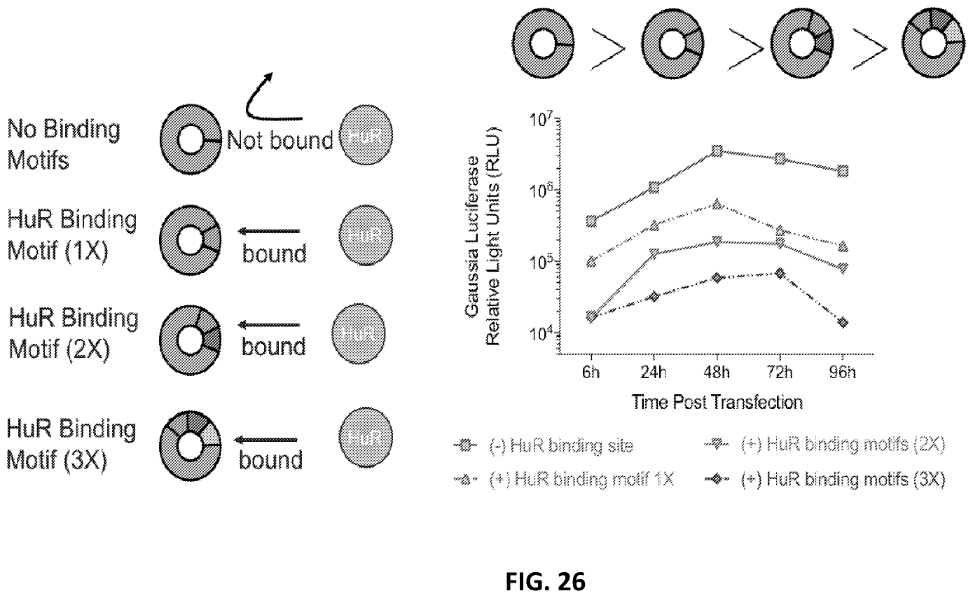

FIG. 26 shows lower secreted protein expression from circular RNA with HuR binding aptamer sites. Even more, the GLuc expression levels changed with the number of HuR binding aptamer motifs in the circular RNA. This example demonstrates that the level of translation from the engineered circular RNA was affected by additional protein binding aptamers.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments described herein can be employed in practicing the disclosure. It is intended that the following claims define the scope of the disclosure and that methods and structures within the scope of these claims and their equivalents be covered thereby.

Sequence listing
SEQ ID NO: 1
AATAGCCGGUCUACGGCCAUACCACCCUGAACGCGC
CCGAUCUCGUCUGAUCUCGGAAGCUAAGCAGGGUCG
GGCCUGGUUAGUACUUGGAUGGGAGACCGCCUGGGA
AUACCGGGUGCUGUAGGCGUCGACUUGCCAUGUGUA
UGUGGGUACGAAGGAAGGAUUGGUAUGUGGUAUAUU
CGUACCCACAUACUCUGAUGAUCCUUCGGGAUCAUU
CAUGGCAACGGCTATT
SEQ ID NO: 2
GGGGGAUCAAUCCAAGGGACCCGGAAACGCUCCCUU
ACACCCC
SEQ ID NO: 3
GGGUGAAUGGUUCUACGAUAAACGUUAAUGACCAGC
UUAUGGCUGGCAGUUCCUAUAGCACCC
SEQ ID NO: 4
TGTTGTGTCTTGGTTGGT
SEQ ID NO: 5
TGTTGTGTGTTGGTTGGT
SEQ ID NO: 6
GTCAACGGATTTTCCCAAGTCCGTAGCGTCTC
SEQ ID NO: 7
CCTGAGATTCCTGGGTTCAAG
SEQ ID NO: 8
CTTCTTGAGCAGGTCAGAACA
SEQ ID NO: 9
GACGAGGCCCAGAGCAAGAGAGG
SEQ ID NO: 10
GGTGTTGAAGGTCTCAAACATG
SEQ ID NO: 11
GGCGUAGUGAUUAUGAAUCGUGUGCUAAUACACGCC
SEQ ID NO: 12
GCGACUGGUUACCCGGUCG
SEQ ID NO: 13
GGTGGTGGTGGTTGTGGTGGTGGTGG
SEQ ID NO: 14
GGTTGAGGGCCACCTTATTT
SEQ ID NO: 15
GAAGAAAGACCGGGAAGAGAAA
SEQ ID NO: 16
GUGAUGGCGCCUACGUCGAAGAAAGGAGUCUCAAGG
GAAGGAGCGUAUAUGGUCGAUGAAUCGGUCAUGUC
GUCAGGGU
SEQ ID NO: 17
GAGUCAUAGGACGCUCGCUCUUGCGACCAUGGGGCA
CGGGGAGCCCACUGCAUGGAUCUAUCGUAUCAUAG
UGCGGU
SEQ ID NO: 18
GUAGCUUCCAUGAGACUUGAUCGGGGUCAUGGCUCU
AGGCAUCGGAGAAGCUGACUAACUUGGUCACGUCG
UACCUGGU
SEQ ID NO: 19
GGACGCGUACGAAGGGCUGAUAGGGCAGAGCUCCAA
CUAUGCGUCCAGCUCGUGCAGUGGAUCGGGUCGUG
CCUGGU
SEQ ID NO: 20
CUUUGUCGGCCGAACUCGCUGUUUAACUGCCCGGCG
AGAUCGCAGGGUGUUGUGCUAUUCGCGUGCCGUGU
G
SEQ ID NO: 21
CCGUAAUACGACUCACUAUAGGGGAGCUCGGUACCG
AAUUCAAGGUACUCUGUGCUUGUCGAUGUGUAUUG
AUGGCACUUUCGAGUCAACGAGUUGACAGAACAAGU
AGUCAAGCUUUGCAGAGAGGAUCCUU
SEQ ID NO: 22
GGGAGAAUUCCGACCAGAAGGGUUAGCAGUCGGCAU
GCGGUACAGACAGACCUUUCCUCucuccuuccucu
ucu
SEQ ID NO: 23
GGGGUAGUGAUUUUACCCUGGAGAU
SEQ ID NO: 24
AATAGCCG
SEQ ID NO: 25
CGGCTATT
SEQ ID NO: 26
TACGCCTGCAACTGTGTTGT
SEQ ID NO: 27
TCGATGATCTTGTCGTCGTC
SEQ ID NO: 28
aaaaaaaaaaGATCTTGAAACTGTTTTAAGGTTGGC
CGATCTTaaaaaa
SEQ ID NO: 29
aaaaaaaTTCTCCGAACGTGTCACGTTTCAAGAGAA
CGTGACACGTTCGGAGAAaaaaaa
SEQ ID NO: 30
UCAUAAUCAAUUUAUUAUUUUCUUUUAUUUUAUUCA
CAUAAUUUUGUUUUU
SEQ ID NO: 31
CGAGACGCTACGGACTTAAAATCCGTTGAC
SEQ ID NO: 32
UCAUAAUCAAUUUAUUAUUUUCUUUUAUUUUAUUCA
CAUAAUUUUGUUUUU
SEQ ID NO: 33
AUUUUGUUUUUAACAUUUC
SEQ ID NO: 34
UCAUAAUCAAUUUAUUAUUUUCUUUUAUUUUAUUCA
CAUAAUUUUGUUUUUAUUUUGUUUUUAACAUUUC

What is claimed is:

1. A parenteral nucleic acid delivery system comprising a circular polyribonucleotide and a parenterally acceptable diluent, wherein the circular polyribonucleotide comprises a sequence that binds a target.

2. The parenteral nucleic acid delivery system of claim 1, wherein the delivery system is free of any carrier.

3. The parenteral nucleic acid delivery system of claim 1, wherein the delivery system comprises a carrier.

4. The parenteral nucleic acid delivery system of any one of claims 1-3, wherein the circular polyribonucleotide is in an amount effective to elicit a biological response in the subject.

5. The parenteral nucleic acid delivery system of any one of claims 1-3, wherein the circular polyribonucleotide is in an amount effective to have a biological effect on a cell or tissue in the subject.

6. The parenteral nucleic acid delivery system of any one of claims 1-5 formulated for intravenous, intramuscular, ophthalmic or topical administration.

7. The parenteral nucleic acid delivery system of any one of claims 1-6 formulated for in vitro or ex vivo delivery to a cell or tissue.

8. The parenteral nucleic acid delivery system of any of the preceding claims, wherein the circular polyribonucleotide forms a complex with the target and the circular polyribonucleotide or the target is detectable at least 5 days after delivery.

9. The parenteral nucleic acid delivery system of any of the preceding claims, wherein the target is selected from the group consisting of a nucleic acid molecule, a small molecule, a protein, a carbohydrate, and a lipid.

10. The parenteral nucleic acid delivery system of claim 9, wherein the small molecule is an organic compound having a molecular weight of no more than 900 daltons and modulates a cellular process.

11. The parenteral nucleic acid delivery system of claim 10, wherein the small molecule is a drug.

12. The parenteral nucleic acid delivery system of claim 10, wherein the small molecule is a fluorophore.

13. The parenteral nucleic acid delivery system of claim 12, wherein the small molecule is a metabolite.

14. The parenteral nucleic acid delivery system of any of claims 1-13, wherein the target is a gene regulation protein.

15. The parenteral nucleic acid delivery system of claim 14, wherein the gene regulation protein is a transcription factor.

16. The parenteral nucleic acid delivery system of claim 9, wherein the nucleic acid molecule is a DNA molecule or an RNA molecule.

17. The parenteral nucleic acid delivery system of any one of claims 8-16, wherein the complex modulates gene expression.

18. The parenteral nucleic acid delivery system of any one of claims 8-17, wherein the complex modulates directed transcription of a DNA molecule, epigenetic remodeling of a DNA molecule, or degradation of a DNA molecule.

19. The parenteral nucleic acid delivery system of any one of claims 8-18 wherein the complex modulates degradation of the target, translocation of the target, or target signal transduction.

20. The parenteral nucleic acid delivery system of any one of claims 17-19, wherein the gene expression is associated with pathogenesis of a disease or condition.

21. The parenteral nucleic acid delivery system of any one of claims 8-20, wherein the circular polyribonucleotide of the complex or the target of the complex is detectable at least 7, 8, 9, or 10 days after delivery.

22. The parenteral nucleic acid delivery system of any one of the preceding claims, wherein the circular polyribonucleotide is present at least five days after delivery.

23. The parenteral nucleic acid delivery system of any one of the preceding claims, wherein the circular polyribonucleotide is present at least 6, 7, 8, 9, or 10 days after delivery.

24. The parenteral nucleic acid delivery system of any one of the preceding claims, wherein the circular polyribonucleotide is an unmodified circular polyribonucleotide.

25. The parenteral nucleic acid delivery system of any one of the preceding claims, wherein the circular polyribonucleotide has a quasi-double-stranded secondary structure.

26. The parenteral nucleic acid delivery system of any one of the preceding claims, wherein the sequence is an aptamer sequence that has a secondary structure that binds the target.

27. The parenteral nucleic acid delivery system of any one of the preceding claims, wherein the aptamer sequence further has a tertiary structure that binds the target.

28. The parenteral nucleic acid delivery system of any one of claims 5-27, wherein the cell is a eukaryotic cell.

29. The parenteral nucleic acid delivery of claim 28, wherein the eukaryotic cell is an animal cell.

30. The parenteral nucleic acid delivery of claim 28, wherein the eukaryotic cells is a pet cell.

31. The parenteral nucleic acid delivery of claim 28, wherein the eukaryotic cell is a mammalian cell.

32. The parenteral nucleic acid delivery of claim 28, wherein the eukaryotic cell is a human cell.

33. The parenteral nucleic acid delivery of claim 28, wherein the eukaryotic cell is a livestock cell.

34. The parenteral nucleic acid delivery system of any one of the preceding claims, wherein the circular polyribonucleotide lacks a poly-A sequence, lacks a replication element, lacks a free 3′ end, or lacks an RNA polymerase recognition motif, or any combination thereof.

35. The parenteral nucleic acid delivery system of any one of the preceding claims, wherein the circular polyribonucleotide is a translation incompetent circular polyribonucleotide.

36. The parenteral nucleic acid delivery system of any one of the claims 1-35, wherein the circular polyribonucleotide further comprises an expression sequence.

37. The system of claim 36, wherein the circular polyribonucleotide comprises a termination element or an IRES, or the combination thereof.

38. The parenteral nucleic acid delivery system of any one of the preceding claims for use as a medicament or a pharmaceutical.

39. The parenteral nucleic acid delivery system of any one of the preceding claims for use in a method of treatment of a human or animal body by therapy.

40. The use of the parenteral nucleic acid delivery system of any one of the preceding claims in the manufacture of a medicament or a pharmaceutical.

41. The use of the parenteral nucleic acid delivery system of any one of the preceding claims in the manufacture of a medicament or a pharmaceutical for treating a human or animal body by therapy.


SEQUENCE LISTING (58 sequences)

SEQ ID NO: 1 - DNA, 232 bp, Artificial Sequence
  source: /note=“Description of Artificial Sequence Synthetic oligonucleotide”
  source: /note=“Description of Combined DNA/RNA Molecule Synthetic oligonucleotide”
aatagccggucuacggccauaccacccugaacgcgcccgaucucgucugaucucggaagcuaagcagggucgggccugguuaguacuuggaugggagaccgccugggaauaccgggugcuguaggcgucgacuugccauguguauguggguacgaaggaaggauugguaugugguauauucguacccacauacucugaugauccuucgggaucauucauggcaacggctatt

SEQ ID NO: 2 - RNA, 43 nt, Artificial Sequence
  source: /note=“Description of Artificial Sequence Synthetic oligonucleotide”
gggggaucaauccaagggacccggaaacgcucccuuacacccc

SEQ ID NO: 3 - RNA, 63 nt, Artificial Sequence
  source: /note=“Description of Artificial Sequence Synthetic oligonucleotide”
gggugaaugguucuacgauaaacguuaaugaccagcuuauggcuggcaguuccuauagcaccc

SEQ ID NO: 4 - DNA, 18 bp, Artificial Sequence
  source: /note=“Description of Artificial Sequence Synthetic oligonucleotide”
tgttgtgtcttggttggt

SEQ ID NO: 5 - DNA, 18 bp, Artificial Sequence
  source: /note=“Description of Artificial Sequence Synthetic oligonucleotide”
tgttgtgtgttggttggt

SEQ ID NO: 6 - DNA, 32 bp, Artificial Sequence
  source: /note=“Description of Artificial Sequence Synthetic oligonucleotide”
gtcaacggattttcccaagtccgtagcgtctc

SEQ ID NO: 7 - DNA, 21 bp, Artificial Sequence
  source: /note=“Description of Artificial Sequence Synthetic primer”
cctgagattcctgggttcaag

SEQ ID NO: 8 - DNA, 21 bp, Artificial Sequence
  source: /note=“Description of Artificial Sequence Synthetic primer”
cttcttgagcaggtcagaaca

SEQ ID NO: 9 - DNA, 23 bp, Artificial Sequence
  source: /note=“Description of Artificial Sequence Synthetic primer”
gacgaggcccagagcaagagagg

SEQ ID NO: 10 - DNA, 22 bp, Artificial Sequence
  source: /note=“Description of Artificial Sequence Synthetic primer”
ggtgttgaaggtctcaaacatg

SEQ ID NO: 11 - RNA, 36 nt, Artificial Sequence
  source: /note=“Description of Artificial Sequence Synthetic oligonucleotide”
ggcguagugauuaugaaucgugugcuaauacacgcc

SEQ ID NO: 12 - RNA, 19 nt, Artificial Sequence
  source: /note=“Description of Artificial Sequence Synthetic oligonucleotide”
gcgacugguuacccggucg

SEQ ID NO: 13 - DNA, 26 bp, Artificial Sequence
  source: /note=“Description of Artificial Sequence Synthetic oligonucleotide”
ggtggtggtggttgtggtggtggtgg

SEQ ID NO: 14 - DNA, 20 bp, Artificial Sequence
  source: /note=“Description of Artificial Sequence Synthetic primer”
ggttgagggccaccttattt

SEQ ID NO: 15 - DNA, 22 bp, Artificial Sequence
  source: /note=“Description of Artificial Sequence Synthetic primer”
gaagaaagaccgggaagagaaa

SEQ ID NO: 16 - RNA, 79 nt, Artificial Sequence
  source: /note=“Description of Artificial Sequence Synthetic oligonucleotide”
gugauggcgccuacgucgaagaaaggagucucaagggaaggagcguauauggucgaugaaucggucaugucgucagggu

SEQ ID NO: 17 - RNA, 77 nt, Artificial Sequence
  source: /note=“Description of Artificial Sequence Synthetic oligonucleotide”
gagucauaggacgcucgcucuugcgaccauggggcacggggagcccacugcauggaucuaucguaucauagugcggu

SEQ ID NO: 18 - RNA, 79 nt, Artificial Sequence
  source: /note=“Description of Artificial Sequence Synthetic oligonucleotide”
guagcuuccaugagacuugaucggggucauggcucuaggcaucggagaagcugacuaacuuggucacgucguaccuggu

SEQ ID NO: 19 - RNA, 77 nt, Artificial Sequence
  source: /note=“Description of Artificial Sequence Synthetic oligonucleotide”
ggacgcguacgaagggcugauagggcagagcuccaacuaugcguccagcucgugcaguggaucgggucgugccuggu

SEQ ID NO: 20 - RNA, 72 nt, Artificial Sequence
  source: /note=“Description of Artificial Sequence Synthetic oligonucleotide”
cuuugucggccgaacucgcuguuuaacugcccggcgagaucgcaggguguugugcuauucgcgugccgugug

SEQ ID NO: 21 - RNA, 133 nt, Artificial Sequence
  source: /note=“Description of Artificial Sequence Synthetic polynucleotide”
ccguaauacgacucacuauaggggagcucgguaccgaauucaagguacucugugcuugucgauguguauugauggcacuuucgagucaacgaguugacagaacaaguagucaagcuuugcagagaggauccuu

SEQ ID NO: 22 - RNA, 74 nt, Artificial Sequence
  source: /note=“Description of Artificial Sequence Synthetic oligonucleotide”
gggagaauuccgaccagaaggguuagcagucggcaugcgguacagacagaccuuuccucucuccuuccucuucu

SEQ ID NO: 23 - RNA, 25 nt, Artificial Sequence
  source: /note=“Description of Artificial Sequence Synthetic oligonucleotide”
gggguagugauuuuacccuggagau

SEQ ID NO: 24 - DNA, 8 bp, Artificial Sequence
  source: /note=“Description of Artificial Sequence Synthetic oligonucleotide”
aatagccg

SEQ ID NO: 25 - DNA, 8 bp, Artificial Sequence
  source: /note=“Description of Artificial Sequence Synthetic oligonucleotide”
cggctatt

SEQ ID NO: 26 - DNA, 20 bp, Artificial Sequence
  source: /note=“Description of Artificial Sequence Synthetic primer”
tacgcctgcaactgtgttgt

SEQ ID NO: 27 - DNA, 20 bp, Artificial Sequence
  source: /note=“Description of Artificial Sequence Synthetic primer”
tcgatgatcttgtcgtcgtc

SEQ ID NO: 28 - DNA, 49 bp, Artificial Sequence
  source: /note=“Description of Artificial Sequence Synthetic oligonucleotide”
aaaaaaaaaagatcttgaaactgttttaaggttggccgatcttaaaaaa

SEQ ID NO: 29 - DNA, 60 bp, Artificial Sequence
  source: /note=“Description of Artificial Sequence Synthetic oligonucleotide”
aaaaaaattctccgaacgtgtcacgtttcaagagaacgtgacacgttcggagaaaaaaaa

SEQ ID NO: 30 - RNA, 51 nt, Artificial Sequence
  source: /note=“Description of Artificial Sequence Synthetic oligonucleotide”
ucauaaucaauuuauuauuuucuuuuauuuuauucacauaauuuuguuuuu

SEQ ID NO: 31 - DNA, 30 bp, Artificial Sequence
  source: /note=“Description of Artificial Sequence Synthetic oligonucleotide”
cgagacgctacggacttaaaatccgttgac

SEQ ID NO: 32 - RNA, 51 nt, Artificial Sequence; identical to SEQ ID NO: 30
  source: /note=“Description of Artificial Sequence Synthetic oligonucleotide”

SEQ ID NO: 33 - RNA, 19 nt, Artificial Sequence
  source: /note=“Description of Artificial Sequence Synthetic oligonucleotide”
auuuuguuuuuaacauuuc

SEQ ID NO: 34 - RNA, 70 nt, Artificial Sequence
  source: /note=“Description of Artificial Sequence Synthetic oligonucleotide”
ucauaaucaauuuauuauuuucuuuuauuuuauucacauaauuuuguuuuuauuuuguuuuuaacauuuc

SEQ ID NO: 35 - RNA, 90 nt, Artificial Sequence
  source: /note=“Description of Artificial Sequence Synthetic oligonucleotide”
cgggucggcauggcaucuccaccuccucgcgguccgaccugggcauccgaaggaggacgcacguccacucggauggcuaagggagagcca

SEQ ID NO: 36 - RNA, 88 nt, Artificial Sequence
  source: /note=“Description of Artificial Sequence Synthetic oligonucleotide”
uggccggcauggucccagccuccucgcuggcgccggcugggcaacauuccgaggggaccguccccucgguaauggcgaaugggaccca

SEQ ID NO: 37 - protein, 9 aa, Artificial Sequence
  source: /note=“Description of Artificial Sequence Synthetic peptide”
  VARIANT (1)..(1): /replace=“His” or “ ”
  VARIANT (2)..(2): /replace=“Gly” or “ ”
  VARIANT (3)..(3): /replace=“Val” or “Ile” or “Ser” or “Met”
  MOD_RES (5)..(5): Any amino acid
  SITE (1)..(9): /note=“Variant residues given in the sequence have no preference with respect to those in the annotations for variant positions”
GDDEXNPGP

SEQ ID NO: 38 - protein, 8 aa, Artificial Sequence
  source: /note=“Description of Artificial Sequence Synthetic peptide”
  VARIANT (2)..(2): /replace=“Ile”
  MOD_RES (4)..(4): Any amino acid
  SITE (1)..(8): /note=“Variant residues given in the sequence have no preference with respect to those in the annotations for variant positions”
DVEXNPGP

SEQ ID NO: 39 - protein, 9 aa, Artificial Sequence
  source: /note=“Description of Artificial Sequence Synthetic peptide”
GDVESNPGP

SEQ ID NO: 40 - protein, 9 aa, Artificial Sequence
  source: /note=“Description of Artificial Sequence Synthetic peptide”
GDIEENPGP

SEQ ID NO: 41 - protein, 7 aa, Artificial Sequence
  source: /note=“Description of Artificial Sequence Synthetic peptide”
VEPNPGP

SEQ ID NO: 42 - protein, 7 aa, Artificial Sequence
  source: /note=“Description of Artificial Sequence Synthetic peptide”
IETNPGP

SEQ ID NO: 43 - protein, 9 aa, Artificial Sequence
  source: /note=“Description of Artificial Sequence Synthetic peptide”
GDIESNPGP

SEQ ID NO: 44 - protein, 9 aa, Artificial Sequence
  source: /note=“Description of Artificial Sequence Synthetic peptide”
GDVELNPGP

SEQ ID NO: 45 - protein, 9 aa, Artificial Sequence
  source: /note=“Description of Artificial Sequence Synthetic peptide”
GDIETNPGP

SEQ ID NO: 46 - protein, 8 aa, Artificial Sequence
  source: /note=“Description of Artificial Sequence Synthetic peptide”
GDVENPGP

SEQ ID NO: 47 - protein, 9 aa, Artificial Sequence
  source: /note=“Description of Artificial Sequence Synthetic peptide”
GDVEENPGP

SEQ ID NO: 48 - protein, 9 aa, Artificial Sequence
  source: /note=“Description of Artificial Sequence Synthetic peptide”
GDVEQNPGP

SEQ ID NO: 49 - protein, 7 aa, Artificial Sequence
  source: /note=“Description of Artificial Sequence Synthetic peptide”
IESNPGP

SEQ ID NO: 50 - protein, 9 aa, Artificial Sequence
  source: /note=“Description of Artificial Sequence Synthetic peptide”
GDIELNPGP

SEQ ID NO: 51 - protein, 9 aa, Artificial Sequence
  source: /note=“Description of Artificial Sequence Synthetic peptide”
HDIETNPGP

SEQ ID NO: 52 - protein, 9 aa, Artificial Sequence
  source: /note=“Description of Artificial Sequence Synthetic peptide”
HDVETNPGP

SEQ ID NO: 53 - protein, 9 aa, Artificial Sequence
  source: /note=“Description of Artificial Sequence Synthetic peptide”
HDVEMNPGP

SEQ ID NO: 54 - protein, 9 aa, Artificial Sequence
  source: /note=“Description of Artificial Sequence Synthetic peptide”
GDMESNPGP

SEQ ID NO: 55 - protein, 9 aa, Artificial Sequence
  source: /note=“Description of Artificial Sequence Synthetic peptide”
GDVETNPGP

SEQ ID NO: 56 - protein, 9 aa, Artificial Sequence
  source: /note=“Description of Artificial Sequence Synthetic peptide”
GDIEQNPGP

SEQ ID NO: 57 - protein, 8 aa, Artificial Sequence
  source: /note=“Description of Artificial Sequence Synthetic peptide”
DSEFNPGP

SEQ ID NO: 58 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
auggaauacucuugguuactt